SEQUENCE LISTING (21 sequences)

SEQ ID NO: 1 - protein, 251 aa, Artifiicial Sequence
  FITCE2 scFv
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVS

SEQ ID NO: 2 - protein, 251 aa, Artificial Sequence
  FITCE2 TyrH133Ala scFv
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFASYMDVWGQGTLVTVS

SEQ ID NO: 3 - protein, 250 aa, Artificial Sequence
  FITCE2 HisH131Ala scFv
VLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGAFYSYMDVWGQGTLVTVS

SEQ ID NO: 4 - protein, 254 aa, Artificial Sequence
  4M5.3 scFv
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 5 - protein, 265 aa, Artificial Sequence
  4D5Flu scFv
DYKDIQMTQSPSSLSASVGDRVTITCRASQSLVHSQGNTYLRWYQQKPGKAPKVLIYKVSNRFSGVPSRFSGSGSGTDFTLTISSLQPEDFATYYCQQSTHVPWTFGQGTKVELKRAGGGGSGGGGSGGGGSSGGGSGGGGSGGGGSEVQLVESGGGLVQPGGSLRLSCAASGFTFSDYWMNWVRQAPGKGLEWVAQIRNKPYNYETYYADSVKGRFTISRDTSKNTVYLQMNSLRAEDTAVYYCTGSYYGMDYWGQGTLVTVSS

SEQ ID NO: 6 - protein, 244 aa, Artificial Sequence
  scFv
DVVMTQTPLSLPVSLGDQASISCRSSQSLVHSQGNTYLRWYLQKPGQSPKVLIYKVSNRFSGVPDRFSGSGSGTDFTLKISRVEAEDLGVYFCSQSTHVPWTFGGGTKLEIGGGGSGGGGSGGGGSEVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSS

SEQ ID NO: 7 - DNA, 789 bp, Artificial Sequence
  anti DNP scFv (VH to VL from rabbit
cagtgtcagcagctggagcagtccggaggaggagccgaaggaggcctggtcaagcctgggggatccctggaactctgctgcaaagcctctggattctccctcagtagtagctactgcatatgttgggtccgccaggctccagggaaggggctggagtggatcggatgcatttatgctggtagtagtggtagcacttactacgcgagctgggtgaatggccgattcactctctccagagacattgaccagagcacaggttgcctacaactgaacagtctgacagccgcggacacggccatgtattactgtgcgagagccccctatagtagtggctgggtcctctactttaacttgtggggcccaggcaccctggtcattgtctcctcaggcggagggggctctggcggcggaggatctgggggagggggcagcccaggtgccacatttgcccaagtgctgacccagactccatcgcctgtgtctgcagctgtgggaggcacagtcaccatcagttgccagtccagtgagagtgtttatggtaacagccgcttagcctggtatcagcagaaaccagggcagtctcccaagctcctgatctattatgcatccactctggcatctggggtcccttcgcggttcaaaggcagtggatctgggacacagttcactctcaccattagcgacctggagtgtgacgatgctgcctcttactactgtcaaggcggttattatagtggtaatcttgatgcgcttgctttcggcggagggaccgaggtggtggtcagaggt

SEQ ID NO: 8 - protein, 161 aa, Artificial Sequence
  anti DNP scFv (VH to VL from rabbit)
CARAPYSSGWVLYFNLWGPGTLVIVSSGGGGSGGGGSGGGGSPGATFAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRG

SEQ ID NO: 9 - DNA, 789 bp, Artificial Sequence
  anti DNP scFv (VL to VH from rabbit)
ccaggtgccacatttgcccaagtgctgacccagactccatcgcctgtgtctgcagctgtgggaggcacagtcaccatcagttgccagtccagtgagagtgtttatggtaacagccgcttagcctggtatcagcagaaaccagggcagtctcccaagctcctgatctattatgcatccactctggcatctggggtcccttcgcggttcaaaggcagtggatctgggacacagttcactctcaccattagcgacctggagtgtgacgatgctgcctcttactactgtcaaggcggttattatagtggtaatcttgatgcgcttgctttcggcggagggaccgaggtggtggtcagaggtggcggagggggctctggcggcggaggatctgggggagggggcagccagtgtcagcagctggagcagtccggaggaggagccgaaggaggcctggtcaagcctgggggatccctggaactctgctgcaaagcctctggattctccctcagtagtagctactgcatatgttgggtccgccaggctccagggaaggggctggagtggatcggatgcatttatgctggtagtagtggtagcacttactacgcgagctgggtgaatggccgattcactctctccagagacattgaccagagcacaggttgcctacaactgaacagtctgacagccgcggacacggccatgtattactgtgcgagagccccctatagtagtggctgggtcctctactttaacttgtggggcccaggcaccctggtcattgtctcctca

SEQ ID NO: 10 - protein, 263 aa, Artificial Sequence
  anti DNP scFv (VL to VH from rabbit)
PGATFAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRGGGGGSGGGGSGGGGSQCQQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIVSS

SEQ ID NO: 11 - protein, 22 aa, Artificial Sequence
  GM-CSF
MLLLVTSLLLCELPHPAFLLIP

SEQ ID NO: 12 - protein, 229 aa, Artificial Sequence
  Spacer (long) IgG4hinge-CH2(L235D)-CH3
ESKYGPPCPPCPAPEFDGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 119 aa, Artificial Sequence
  Spacer (medium) IgG4 hinge-CH3
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 12 aa, Artificial Sequence
  Spacer (short) IgG4 hinged
ESKYGPPCPPCP

SEQ ID NO: 15 - protein, 28 aa, Artificial Sequence
  CD28tm
MFWVLVVVGGVLACYSLLVTVAFIIFWV

SEQ ID NO: 16 - protein, 42 aa, Artificial Sequence
  4-1BB
KRGRKKLLYIFKQPFMRPVQTTQEEDGCSCRFPEEEEGGCEL

SEQ ID NO: 17 - protein, 112 aa, Artificial Sequence
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 21 aa, Artificial Sequence
  T2A
GGGEGRGSLLTCGDVEENPGP

SEQ ID NO: 19 - protein, 357 aa, Artificial Sequence
  GM-CSF receptor ss to EGFRt
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 20 - protein, 187 aa, Artificial Sequence
  DHFRdm
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 21 - protein, 1751 aa, Artificial Sequence
  Dual CAR sequence mAb806 VHVL scFv-IgG4hinge-CD28tm/CD28gg-Zeta-T2A-EGFRt-P2-Ant iFL(FITC-E2 Tyr100gAla)scFv-IgG4hinge-CH2(L235D, N297Q)-CH3--CD28tm/41BB-zeta-T2A-DHFRdm-epHIV7.2
MLLLVTSLLLCELPHPAFLLIPDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGSTSGSGKPGSGEGSTKGDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRESKYGPPCPPCPMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGSGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGATNFSLLKQAGDVEENPGPMLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFASYMDVWGQGTLVTVSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

(19) United States
(12) Patent Application Publication
(10) Pub. No.: US 2023/0172981 A1
(43) Pub. Date: Jun. 8, 2023

(54) METHODS AND COMPOSITIONS FOR STIMULATION OF CHIMERIC ANTIGEN RECEPTOR T CELLS WITH HAPTEN LABELLED CELLS

(71) Applicant: Seattle Children's Hospital (dba Seattle Children's Research Institute), Seattle, WA (US)

(72) Inventors: Michael C. Jensen, Seattle, WA (US); James F. Matthaei, Seattle, WA (US)

(21) Appl. No.: 17/758,960

(22) PCT Filed: Feb. 2, 2021
(86) PCT No.: PCT/US2021/016194

§ 371 Date: Jul. 15, 2022

Related U.S. Application Data
(60) Provisional application No. 62/969,917, filed on Feb. 4, 2020.

(51) Int. Cl. A61K 35/17; C07K 14/725

(52) U.S. Cl. CPC A61K 35/17; C07K 14/7051

(57) ABSTRACT

Some embodiments of the methods and compositions provided herein relate to the use of hapten labeled cells to stimulate chimeric antigen receptor (CAR) T cells. In some embodiments, CAR T cells can include a CAR that specifically binds to a hapten. Some embodiments relate to the in vivo or in vitro stimulation CAR T cells by hapten labeled cells.

### CROSS-REFERENCE TO RELATED APPLICATIONS

This application is a U.S. National Phase Application of PCT International Application Number PCT/US2021/016194, filed on Feb. 2, 2021, designating the United States of America and published in the English language, which is an International Application of and claims the benefit of priority to U.S. Provisional Application No. 62/969,917, filed on Feb. 4, 2020. The disclosures of the above-referenced applications are hereby expressly incorporated by reference in their entireties.

### REFERENCE TO SEQUENCE LISTING

The present application is being filed along with a Sequence Listing in electronic format. The Sequence Listing is provided as a file entitled SCRI272NPSEQLIST, created Jan. 2, 2023, which is approximately 44505 bytes in size. The information in the electronic format of the Sequence Listing is incorporated herein by reference in its entirety.

### FIELD

Some embodiments of the methods and compositions provided herein relate to the use of hapten labeled cells to stimulate chimeric antigen receptor (CAR) T cells. In some embodiments, the CAR T cells include a CAR that specifically binds to a hapten. Some embodiments also relate to the in vivo or in vitro stimulation of CAR T cells by hapten labeled cells.

### BACKGROUND

Immunotherapy using the adoptive cell transfer (ACT) of chimeric antigen receptor bearing T-cells has been previously described for use in treating cancer. The structure of a chimeric antigen receptor (CAR) includes antigen binding domains, linker and spacer sequences, co-stimulatory activation domains and transmembrane regions. The cells expressing the CAR may be from the patient in need of treatment or a donor cell (relative or non-relative). The CAR functions by attaching to a specific protein or antigen on a cell or tumor cell. Infusion of the CAR T cells into the patient leads to the engineered cells being further multiplied into the patient's body, which will recognize and kill the cells that have the specific protein or antigen on the cancer cell or tumor cell surface.

It is desirable that the CAR T cells maintain potency over time. CAR T cell populations contract and lose potency once hematologic cancers reach final stages of regression due to low cancer cell levels and thus, lowered levels of the antigen. Additionally, solid tumors are also very immunosuppressive within their tumor environment. Thus, CAR T cells can require additional stimulation to remove the residual cancer cells that are left in order to complete the therapy. Stimulation and re-stimulation may also be used to overcome an immunosuppressive tumor environment.

Stimulation and re-stimulation of CAR T cells have been previously described. Stimulation of cells can be performed in vitro by the addition of antiCD3/CD28 beads prior to infusion into a patient, for example. The alternatives provided herein describe new approaches to stimulate CAR T cells both in vivo and in vitro.

### SUMMARY

Some embodiments of the methods and compositions provided herein include approaches for inducing expansion of a chimeric antigen receptor (CAR) T cell comprising: incubating the CAR T cell with a hapten antigen presenting cell (H-APC), wherein a CAR of the CAR T cell specifically binds to a hapten attached to the H-APC. In some embodiments, the CAR T cell and the H-APC are derived from a single subject, such as a mammal, preferably a human.

Some embodiments of the methods and compositions provided herein include methods of treating, inhibiting, or ameliorating a cancer in a subject comprising: administering an effective amount of a chimeric antigen receptor (CAR) T cell to the subject, wherein a CAR of the CAR T cell specifically binds to a tumor specific antigen of the cancer; and inducing expansion of the CAR T cell by incubating the CAR T cell with a hapten antigen presenting cell (H-APC), wherein a CAR of the CAR T cell specifically binds to a hapten attached to the H-APC. In some embodiments, the CAR T cell and the H-APC are derived from the subject, such as a human.

In some embodiments, the CAR T-cell comprises a bispecific CAR.

In some embodiments, the CAR T-cell comprises more than one CARs.

In some embodiments, the CAR T cell comprises a first ligand binding domain, which can specifically bind to a tumor specific antigen, and a second ligand binding domain, which can specifically bind to the hapten.

In some embodiments, the CAR T-cell comprises a monospecific CAR. In some embodiments, the CAR comprises a single ligand binding domain, which can specifically bind to a tumor specific antigen and to the hapten.

In some embodiments, the incubation is in vitro.

In some embodiments, the incubation is in vivo.

In some embodiments, the CAR specifically binds a tumor specific antigen. In some embodiments, the tumor specific antigen is selected from the group consisting of CD19, CD22, HER2, CD7, CD30, B cell maturation antigen (BCMA), GD2, glypican-3, MUC1, CD70, CD33, epithelial cell adhesion molecule (EpCAM), Epidermal Growth Factor variant III, receptor tyrosine kinase-like orphan receptor 1 (ROR1), CD123, Prostate Stem Cell Antigen (PSCA), CD5, Lewis Y antigen, B7H3, CD20, CD43, HSP90, and IL13.

In some embodiments, the hapten is selected from a hapten listed in TABLE 1 or a ligand binding domain comprises a binding fragment of an antibody selected from an antibody against a hapten listed in TABLE 1 or an antibody listed in TABLE 2 or a sequence from TABLE 3 or a CAR comprises one or more of the sequences of TABLE 4. In some embodiments, the hapten is selected from fluorescein, urushiol, quinone, biotin, or dinitrophenol, or derivatives thereof. In some embodiments, the hapten is selected from fluorescein, dinitrophenol, or derivatives thereof.

In some embodiments, the hapten is covalently attached to the extracellular surface of the H-APC. In some embodiments, the hapten is attached to the H-APC via a phospholipid ether (PLE).

In some embodiments, the CAR T cell is derived from a CD4+ cell or a CD8+ cell.

In some embodiments, the CD8+ cell is a CD8+T cytotoxic lymphocyte cell selected from the group consisting of naïve CD8+ T cells, central memory CD8+ T cells, effector memory CD8+ T cells, and bulk CD8+ T cells. In some embodiments, the CD8+ cell is a CD8+ cytotoxic T lymphocyte cell is a central memory T cell and, wherein the central memory T cell is positive for CD45RO+, CD62L+, and CD8+.

In some embodiments, the CD4+ cell is a CD4+T helper lymphocyte cell selected from the group consisting of naïve CD4+ T cells, central memory CD4+ T cells, effector memory CD4+ T cells, and bulk CD4+ T cells. In some embodiments, the CD4+ helper lymphocyte cell is a naïve CD4+ T cell and, wherein the naïve CD4+ T cell is positive for CD45RA+, CD62L+ and CD4+ and negative for CD45RO.

In some embodiments, the CAR T cell is derived from a precursor T cell. In some embodiments, the CAR T cell is derived from hematopoietic stem cell.

In some embodiments, the H-APC is derived from a cell selected from the group consisting of a T cell, and a B cell.

In some embodiments, the subject is mammalian, such as a livestock animal or domestic animal. In some embodiments, the subject is human.

Some embodiments of the methods and compositions provided herein include a composition comprising one or more nucleic acids encoding a first chimeric antigen receptor (CAR) and a second chimeric antigen receptor (CAR), the one or more nucleic acids comprising: a first sequence encoding the first CAR, wherein the first CAR comprises a first ligand binding domain, which is specific for a tumor antigen, a first polypeptide spacer, a first transmembrane domain and a first intracellular signaling domain; and a second sequence encoding the second CAR, wherein the second CAR comprises a second ligand binding domain specific for a hapten, a second polypeptide spacer, a second transmembrane domain and a second intracellular signaling domain.

In some embodiments, the first ligand binding domain specifically binds an antigen selected from the group consisting of CD19, CD22, HER2, CD7, CD30, B cell maturation antigen (BCMA), GD2, glypican-3, MUC1, CD70, CD33, epithelial cell adhesion molecule (EpCAM), Epidermal Growth Factor variant III, receptor tyrosine kinase-like orphan receptor 1 (ROR1), CD123, Prostate Stem Cell Antigen (PSCA), CD5, Lewis Y antigen, B7H3, CD20, CD43, HSP90, and IL13.

In some embodiments, the hapten is selected from a hapten listed in TABLE 1 or a ligand binding domain comprises a binding fragment of an antibody selected from an antibody against a hapten listed in TABLE 1 or an antibody listed in TABLE 2 or a sequence from TABLE 3 or a CAR comprises one or more of the sequences of TABLE 4. In some embodiments, the hapten is selected from fluorescein, urushiol, quinone, biotin, or dinitrophenol, or derivatives thereof. In some embodiments, the hapten is selected from fluorescein, dinitrophenol, or derivatives thereof.

In some embodiments, the first and/or second ligand binding domain comprises an antibody or binding fragment thereof or scFv. In some embodiments, the second ligand binding domain comprises a binding fragment of an antibody selected from an antibody against a hapten listed in TABLE 1, or an antibody listed in TABLE 2 or a sequence from TABLE 3 or a CAR comprises one or more of the sequences of TABLE 4.

In some embodiments, the first polypeptide spacer and/or second polypeptide spacer comprises a length of 1-24, 25-50, 51-75, 76-100, 101-125, 126-150, 151-175, 176-200, 201-225, 226-250 or 251-275 amino acids.

In some embodiments, the nucleic acid further comprises a leader sequence.

In some embodiments, the first and/or second intracellular signaling domains comprises CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83 or CD3-zeta cytoplasmic domains or both.

In some embodiments, the intracellular signaling domain comprises a portion of CD3 zeta and a portion of 4-1BB.

Some embodiments also include a sequence encoding a marker sequence. In some embodiments, the marker is EGFRt, CD19t, or Her2tG.

In some embodiments, the first and/or second transmembrane domain comprises the transmembrane domain of CD28.

In some embodiments, the one or more nucleic acids further comprises a sequence encoding a cleavable linker.

In some embodiments, the linker is a ribosome skip sequence. In some embodiments, the ribosome skip sequence is P2A, T2A, E2A or F2A.

Some embodiments of the methods and compositions provided herein include a vector comprising the composition of certain embodiments provided herein.

Some embodiments of the methods and compositions provided herein include a composition comprising one or more nucleic acids encoding a first chimeric antigen receptor (CAR) and a second chimeric antigen receptor (CAR), comprising: a first nucleic acid comprising a first sequence encoding the first CAR, wherein the first chimeric antigen receptor comprises a first ligand binding domain, which is specific for a tumor antigen, a first polypeptide spacer, a first transmembrane domain and a first intracellular signaling domain; and a second nucleic acid comprising a second sequence encoding the second CAR, wherein the second chimeric antigen receptor comprises a second ligand binding domain, which is specific for a hapten, a second polypeptide spacer, a second transmembrane domain and a second intracellular signaling domain.

In some embodiments, the first ligand binding domain specifically binds to an antigen selected from the group consisting of CD19, CD22, HER2, CD7, CD30, B cell maturation antigen (BCMA), GD2, glypican-3, MUC1, CD70, CD33, epithelial cell adhesion molecule (EpCAM), Epidermal Growth Factor variant III, receptor tyrosine kinase-like orphan receptor 1 (ROR1), CD123, Prostate Stem Cell Antigen (PSCA), CD5, Lewis Y antigen, B7H3, CD20, CD43, HSP90, and IL13

In some embodiments, the hapten is selected from a hapten listed in TABLE 1 or a ligand binding domain comprises a binding fragment of an antibody selected from an antibody against a hapten listed in TABLE 1 or an antibody listed in TABLE 2 or a sequence from TABLE 3 or a CAR comprises one or more of the sequences of TABLE 4. In some embodiments, the hapten is selected from fluorescein, urushiol, quinone, biotin, or dinitrophenol, or derivatives thereof. In some embodiments, the hapten is selected from fluorescein, or dinitrophenol, or derivatives thereof.

In some embodiments, the first and/or second ligand binding domain comprises an antibody or binding fragment thereof or scFv. In some embodiments, the second ligand binding domain comprises a binding fragment of an antibody selected from an antibody against a hapten listed in TABLE 1 or an antibody listed in TABLE 2 or a sequence from TABLE 3 or a CAR comprises one or more of the sequences of TABLE 4.

In some embodiments, the first polypeptide spacer and/or second polypeptide spacer comprises a length of 1-24, 25-50, 51-75, 76-100, 101-125, 126-150, 151-175, 176-200, 201-225, 226-250 or 251-275 amino acids.

In some embodiments, the one or more nucleic acids further comprise a leader sequence.

In some embodiments, the first and/or second intracellular signaling domains comprises CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83 or CD3-zeta cytoplasmic domains or both.

In some embodiments, the intracellular signaling domain comprises a portion of CD3 zeta and a portion of 4-1BB.

Some embodiments also include a sequence encoding a marker sequence. In some embodiments, the marker is EGFRt, CD19t, or Her2tG.

In some embodiments, the first and/or second transmembrane domain comprises the transmembrane domain of CD28.

In some embodiments, the nucleic acids further comprise a sequence encoding a cleavable linker.

In some embodiments, the linker is a ribosome skip sequence. In some embodiments, the ribosome skip sequence is P2A, T2A, E2A or F2A.

Some embodiments of the methods and compositions provided herein include or utilize a plurality of vectors, such as two vectors, comprising the one or more nucleic acids of any one embodiment provided herein.

Some embodiments of the methods and compositions provided herein include a composition comprising one or more nucleic acids encoding a bispecific chimeric antigen receptor (CAR), the one or more nucleic acids comprising: a sequence encoding a first ligand binding domain, which is specific for a tumor antigen, a Gly-Ser linker, a second ligand binding domain specific for a hapten, a polypeptide spacer, a transmembrane domain and intracellular signaling domain.

In some embodiments, the first ligand binding domain specifically binds to an antigen selected from the group consisting of CD19, CD22, HER2, CD7, CD30, B cell maturation antigen (BCMA), GD2, glypican-3, MUC1, CD70, CD33, epithelial cell adhesion molecule (EpCAM), Epidermal Growth Factor variant III, receptor tyrosine kinase-like orphan receptor 1 (ROR1), CD123, Prostate Stem Cell Antigen (PSCA), CD5, Lewis Y antigen, B7H3, CD20, CD43, HSP90, and IL13.

In some embodiments, the hapten is selected from a hapten listed in TABLE 1 or a ligand binding domain comprises a binding fragment of an antibody selected from an antibody against a hapten listed in TABLE 1 or an antibody listed in TABLE 2 or a sequence from TABLE 3 or a CAR comprises one or more of the sequences of TABLE 4. In some embodiments, the hapten is selected from fluorescein, urushiol, quinone, biotin, or dinitrophenol, or derivatives thereof. In some embodiments, the hapten is selected from fluorescein, or dinitrophenol, or derivatives thereof.

In some embodiments, the first and/or second ligand binding domain comprises an antibody or binding fragment thereof or scFv. In some embodiments, the second ligand binding domain comprises a binding fragment of an antibody selected from an antibody against a hapten listed in TABLE 1, or an antibody listed in TABLE 2 or a sequence from TABLE 3 or a CAR comprises one or more of the sequences of TABLE 4.

In some embodiments, the first polypeptide spacer and/or second polypeptide spacer comprises a length of 1-24, 25-50, 51-75, 76-100, 101-125, 126-150, 151-175, 176-200, 201-225, 226-250 or 251-275 amino acids.

In some embodiments, the one or more nucleic acid further comprises a leader sequence.

In some embodiments, the intracellular signaling domain comprises CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83 or CD3-zeta cytoplasmic domains or both. In some embodiments, the intracellular signaling domain comprises a portion of CD3 zeta and a portion of 4-1BB.

Some embodiments also include a sequence encoding a marker sequence. In some embodiments, the marker is EGFRt, CD19t, or Her2tG.

In some embodiments, the transmembrane domain comprises the transmembrane domain of CD28.

Some embodiments of the methods and compositions provided herein include a vector for bispecific CAR expression comprising the one or more nucleic acids of any one embodiment provided herein.

Some embodiments of the methods and compositions provided herein include a bi-specific chimeric antigen receptor encoded by the one or more nucleic acids of any one of embodiment provided herein or the vector of any one embodiment provided herein.

Some embodiments of the methods and compositions provided herein include a cell comprising the one or more nucleic acids of any one embodiment provided herein, the one or more vectors of embodiment provided herein, or the bi— specific chimeric antigen receptor of any one embodiment provided herein.

In some embodiments, the cell is a CD8+T cytotoxic lymphocyte cell selected from the group consisting of naïve CD8+ T cells, central memory CD8+ T cells, effector memory CD8+ T cells and bulk CD8+ T cells. In some embodiments, the CD8+ cytotoxic T lymphocyte cell is a central memory T cell and, wherein the central memory T cell is positive for CD45RO+, CD62L+, and CD8+.

In some embodiments, the cell is a CD4+T helper lymphocyte cell selected from the group consisting of naïve CD4+ T cells, central memory CD4+ T cells, effector memory CD4+ T cells, and bulk CD4+ T cells. In some embodiments, the cell is a naïve CD4+ T cell and, wherein the naïve CD4+ T cell is positive for CD45RA+, CD62L+ and CD4+ and negative for CD45RO.

In some embodiments, the cell is a precursor T cell. In some embodiments, the cell is a hematopoietic stem cell.

Some embodiments of the methods and compositions provided herein include a method of making a cell that expresses a first chimeric antigen receptor, which is specific for a hapten, and a second chimeric antigen receptor, which is specific for a tumor antigen, the method comprising: introducing the one or more nucleic acids of any one embodiment provided herein or the one or more vectors of certain embodiments provided herein into a cell under conditions whereby the first and second chimeric antigen receptor are expressed.

In some embodiments, the cell is a CD8+T cytotoxic lymphocyte cell selected from the group consisting of naïve CD8+ T cells, central memory CD8+ T cells, effector memory CD8+ T cells and bulk CD8+ T cells. In some embodiments, the CD8+ cytotoxic T lymphocyte cell is a central memory T cell and, wherein the central memory T cell is positive for CD45RO+, CD62L+, and CD8+.

In some embodiments, the cell is a CD4+T helper lymphocyte cell selected from the group consisting of naïve CD4+ T cells, central memory CD4+ T cells, effector memory CD4+ T cells, and bulk CD4+ T cells. In some embodiments, the CD4+ helper lymphocyte cell is a naïve CD4+ T cell and, wherein the naïve CD4+ T cell is positive for CD45RA+, CD62L+ and CD4+ and negative for CD45RO.

In some embodiments, the cell is a precursor T cell. In some embodiments, the cell is a hematopoietic stem cell.

Some embodiments of the methods and compositions provided herein include a method of making a cell that expresses a bispecific chimeric antigen receptor, which is specific for a hapten and a tumor antigen, the method comprising: introducing the one or more nucleic acids of certain embodiments provided herein or the one or more vector of certain embodiments provided herein into a cell under conditions whereby the first and second chimeric antigen receptor are expressed.

In some embodiments, the cell is a CD8+T cytotoxic lymphocyte cell selected from the group consisting of naïve CD8+ T cells, central memory CD8+ T cells, effector memory CD8+ T cells and bulk CD8+ T cells. In some embodiments, the CD8+ cytotoxic T lymphocyte cell is a central memory T cell and, wherein the central memory T cell is positive for CD45RO+, CD62L+, and CD8+.

In some embodiments, the cell is a CD4+T helper lymphocyte cell selected from the group consisting of naïve CD4+ T cells, central memory CD4+ T cells, effector memory CD4+ T cells, and bulk CD4+ T cells. In some embodiments, the CD4+ helper lymphocyte cell is a naïve CD4+ T cell and, wherein the naïve CD4+ T cell is positive for CD45RA+, CD62L+ and CD4+ and negative for CD45RO.

In some embodiments, the cell is a precursor T cell. In some embodiments, the cell is a hematopoietic stem cell.

Some embodiments of the methods and compositions provided herein include a method of stimulating or re-stimulating chimeric antigen receptor (CAR)— bearing T-cells in a subject, preferably a human, suffering from a disease, such as cancer, the method comprising: providing or administering the cell of any one of certain embodiments provided herein to the subject; monitoring the subject for inhibition of said disease; and providing hapten antigen presenting cells (H-APC) to the subject, wherein said subject is optionally, selected or identified to receive a CAR T cell therapy utilizing CAR T cells having receptors specific for an antigen associated with the disease, such as a tumor antigen. Such a selection or identification can be made using clinical and diagnostic evaluation or both.

In some embodiments, the H-APC is generated from healthy cells of the subject by ex vivo labeling the healthy cells with a hapten.

In some embodiments, the hapten is selected from a hapten listed in TABLE 1.

In some embodiments, the monitoring and the providing or administering steps are repeated.

In some embodiments, the subject has a cancer. In some embodiments, the cancer is solid tumor. In some embodiments, the subject, such as a human, is selected or identified to receive a cancer therapy e.g., by clinical or diagnostic evaluation or both. In some embodiments, the subject, such as a human, is subjected to combination therapy, such as chemotherapy or radiation.

Some embodiments of the methods and compositions provided herein include a method of stimulating or re-stimulating chimeric antigen receptor (CAR)— bearing T-cells ex vivo, the method comprising: providing the cell of certain embodiments provided herein; providing hapten antigen presenting cells (H-APC) or a hapten; mixing the cell and the H-APC cells, thereby making activated cells; and isolating the activated cells. In some embodiments, the hapten is selected from a hapten listed in TABLE 1. In some embodiments, the H-APC comprises a hapten selected from a hapten listed in TABLE 1. In some embodiments, isolating the activated cells comprises affinity isolation with hapten complexed affinity beads. In some embodiments, isolating the activated cells comprises affinity isolation with EGFRt, CD19t, or Her2tG complexed affinity beads.

### BRIEF DESCRIPTION OF THE DRAWINGS

FIG. 1A is a schematic view of three chimeric antigen receptors (CARs). Panel (1) depicts a second generation CAR with an antigen recognition moiety (i) which is presented at a desired distance by a spacer domain (ii) from the cell surface. The spacer is connected to a transmembrane domain (iii) which is connected to two signaling domains (iv, and v). Panel (2) depicts a CAR with an extended/longer spacer. This CAR has a different antigen recognition moiety (vi) from the CAR of panel (i) and a longer spacer domain (vii). Panel (3) depicts a bispecific CAR that contains two antigen recognition domains which are linked together. This CAR can activate through the recognition of either epitope.

FIG. 1B is a schematic view of a CAR T cell containing two different CARs (a dual CAR T cell).

FIG. 1C is a schematic view of a CAR T cell containing a bispecific CAR. A bispecific CAR T cell expresses one CAR that can recognize two different epitopes.

FIG. 2 is a schematic of example embodiment of a therapy. Hapten antigen presenting cells (H-APC) are prepared by loading a healthy cell with a hapten on the surface of the cell. These H-APC are then infused into a patient. A dual CAR T cell and bispecific CAR T cells (FIG. 1C) can be activated by recognition of the tumor cell or through the H-APC. One CAR (i) is designed to target an epitope on the tumor cell (ii) whereas the other CAR (iii) is engineered to recognize the hapten (iv) on the Hapten-APC. The Hapten-APC is generated by loading a healthy cell with a hapten on the surface of the cell. These Hapten-APC are then infused back into the patient where they can be recognized and lysed causing the CAR T cells to activate. If these cells are not lysed by CAR T cells the hapten will be metabolized and the Hapten-APC will return to a normal healthy cell. Note, that in some embodiments a single antihapten CAR T cell is used if e.g., a tumor cell is labeled with the same hapten that the hapten-APC is labeled with.

FIG. 3A depicts a structure of a hapten, fluorescein, linked to a phospholipid ether (FL-PLE). The structure includes: (i) a fluorescein moiety; (ii) a polyethene glycol (PEG) moiety which is a spacer which can extend the hapten from a cell surface; (iii) a polar head moiety; and (iv) a hydrophobic tail moiety which is incorporated into a cell plasma membrane.

FIG. 3B depicts the structure of N-(Fluorescein-5-Thiocarbamoyl)-1,2-Dihexadecanoyl-sn-Glycero-3-Phosphoethanolamine (FL-DHPE)

FIG. 3C depicts the structure of N-(4,4-Difluoro-5,7-Dimethyl-4-Bora-3a,4a-Diaza-s-Indacene-3-Propionyl)-1,2-Dihexadecanoyl-sn-Glycero-3-Phosphoethanolamine (Bodipy-DHPE) which includes a hapten, bopidy.

FIG. 4A depicts the results of flow cytometry after incubating CD19+ Raji cells with either FL-DHPE, or an antiCD19-FITC antibody.

FIG. 4B depicts the results of flow cytometry after incubating K562 cells with either 0.5 μM or 5 μM FL-PLE.

FIG. 4C depicts the results of flow cytometry after incubating Be2 cells, U87 cells, or daoy cells with 5 μM FL-PLE.

FIG. 5A is an embodiment of a confocal image of U87 cells that had been incubated with 5 μM FL-PLE and stained with DAPI.

FIG. 5B is an embodiment of a confocal image of U87 cells that had been incubated with 5 μM FL-PLE and stained with DAPI, and with an antifluorescein antibody conjugated with an Alexa Fluor 647 fluorophore.

FIG. 6A depicts the results of incubating Be2 cells or U87 cells with 5 μM FL-DHPE, then measuring the retention of signal over a period of time.

FIG. 6B depicts the results of incubating Be2 cells or U87 cells with 5 μM FL-PLE, then measuring the retention of signal over time.

FIG. 7A is a series of graphs depicting a cytotoxic assay. A chromium release assay was used to test the lytic capabilities of two antiFL CAR T cells (4M5.3 and FITC-E2) against hapten-labelled cells. To generate, hapten labeled cells, CD19+K562 cells were incubated with 5 μM FL-DHPE or stained with an antiCD19-FITC antibody. OKT3 cells were used as a positive control which provides endogenous activation of T cells through the TCR.

FIG. 7B is a series of graphs depicting measurement of cytokine generation by cytokine release assays. The two antiFL CAR T cells (4M5.3 and FITC-E2) were tested against hapten-labelled cells generated by either incubating CD19+K562 cells with 5 μM FL-DHPE, or with an antiCD19-FITC antibody.

FIG. 8A depicts the results of flow cytometry after incubating K562 cells with either 0.5 μM or 5 μM FL-PLE.

FIG. 8B is a series of graphs depicting a cytotoxic assay for antiFL CAR T cells incubated with hapten labeled cells prepared by incubating K562 cells with either 0.5 μM or 5 μM FL-PLE. K562 parental cells were used as a negative control. K562+OKT3 was used as a positive control.

FIG. 8C is a series of graphs depicting measurement of cytokine generation by cytokine release assays for antiFL CAR T cells incubated with hapten labeled cells prepared by incubating K562 cells with either 0.5 μM or 5 μM FL-PLE.

FIG. 9A depicts the results of a flow cytometric analysis showing that antiFL CAR T cells express similar phenotypic markers whether subjected to FREP or REP.

FIG. 9B depicts the results of a flow cytometric analysis after incubating K562 cells with 5 μM FL-PLE in the presence or absence of fetal bovine serum (FBS).

FIG. 9C is a series of graphs depicting a cytotoxic assay for antiFL CAR T cells subjected to FREP or REP. The cells collected from FIG. 9B were used in these assays.

FIG. 9D is a series of graphs depicting cytokine stimulation for antiFL CAR T cells subjected to FREP or REP. The cells collected from FIG. 9B were used in these assays.

FIG. 10A shows a depiction of a structure of a phospholipid ether tethered to the hapten, 2,4-dinitrophenol, (DNP-PLE) [shown as (i)], the target for CAR T cells. Shown as (ii) is polyethene glycol (PEG), the spacer used to extend the target an ideal distance from the cell surface. Shown as (iii) & (iv) PLE, (iii) is the polar head group, and (iv) is the hydrophobic tail for incorporation or tethering into the cell plasma membrane.

FIG. 10 B shows an NMR graph showing the correct structure of DNP-PLE.

FIG. 11A-FIG. 11E show data related to generation of cells with tethered extracellular exposed haptens specifically DNP using DNP-PLE.

FIG. 11A shows flowcytometry data of MDA-MB-231 parentals and MDA-MB-231 cells stained with the antiDNP-Alexa Fluor 488 antibody only.

FIG. 11B shows flowcytometry data of MDA-MB-231 parentals and MDA-MB-231 cells incubated with 5 μM DNP-PLE and stained with the antiDNP-Alexa Fluor 488 antibody.

FIG. 11C shows flowcytometry data of MDA-MB-231 parentals and MDA-MB-231 cells incubated with 500 nM DNP-PLE and stained with the antiDNP-Alexa Fluor 488 antibody.

FIG. 11D shows flowcytometry data of MDA-MB-231 parentals and MDA-MB-231 cells incubated with 50 nM DNP-PLE and stained with the antiDNP-Alexa Fluor 488 antibody.

FIG. 11E shows histogram plots for the flowcytomerty data in FIG. 11A-FIG. 11D.

FIG. 12A-FIG. 12D show confocal microscopy data related to integration of DNP-PLE into cells.

FIG. 12A shows confocal images of MDA-MB-231 parental cells without DNP-PLE but with antiDNP-Alexa Fluor 488 antibody.

FIG. 12B shows confocal images of MDA-MB-231 parental incubated with 5 μM DNP-PLE and without antiDNP-Alexa Fluor 488 antibody.

FIG. 12C shows confocal images of MDA-MB-231 parental incubated with 5 μM DNP-PLE and stained with antiDNP-Alexa Fluor 488 antibody.

FIG. 12D shows confocal images of MDA-MB-231 parental incubated with 1 μM DNP-PLE and stained with antiDNP-Alexa Fluor 488 antibody.

FIG. 13A-FIG. 13D show data related to confirmation of extracellular accessibility of loaded hapten on a cell and that the PLE is loading in membrane.

FIG. 13A shows a schematic of a second generation long CAR cassette for an antiDNP CAR.

FIG. 13B shows flow cytometry data showing populations of H9 parentals, H9 parentals stained with Erbitux antibody, and antiDNP CAR H9 cells stained with Erbitux antibody.

FIG. 13C shows confocal images of MDA-MB-231 co-cultured with antiDNP CAR H9 cells.

FIG. 13D shows confocal images MDA-MB-231 loaded with 504 DNP-PLE co-cultured with antiDNP CAR H9 cells.

FIG. 14 shows graphs of data related to cytokine production by CD19 CAR-T cells with multiple target cells and non-autologous T-APCs.

FIG. 15A-FIG. 15C show data related to autologous T-APC activation in vitro.

FIG. 15A shows detection of expression of CD19t and truncated EGFR (EGFRt) on the cell surface clinically manufactured mixed CD4+/CD8+ truncated CD19 (CD19t) Transduced-Antigen Presenting Cells (T-APC) by flow cytometry.

FIG. 15B shows detection of EGFRt expression on CD4+ and CD8+ transduced CD19 CAR T cells by flow cytometry.

FIG. 15C shows graphs related to cytokine production by CD4+ and CD8+ transduced CD19 CAR T cells.

FIG. 16A-FIG. 16C show data related to autologous Hapten-APC activation in vitro.

FIG. 16A shows analysis of fluorescence by flow cytometry of K562 leukemia cells incubated overnight with or without 5 μM FL-PLE.

FIG. 16B shows shows analysis of fluorescence by flow cytometry of primary CD8+ T cells incubated overnight with or without 5 μM FL-PLE.

FIG. 16C shows graphs related to cytokine production by activated antiFL CAR T cells.

FIG. 17A-FIG. 17D show data related to CAR T cell persistence in peripheral blood (PB) of two pediatric patients and the use of T-APC to stimulate the CAR T cells.

FIG. 17A shows a graph related to the status of CAR T cells, T-APC, and CD19+ B cells populations in the peripheral blood after treatment in a patient.

FIG. 17B shows a graph related to the status of CAR T cells, T-APC, and CD19+ B cells populations in the peripheral blood after treatment in a second patient.

FIG. 17C shows detection of CAR T cells by flow cytometry in the second patient of FIG. 17B at C1.T2.D1.

FIG. 17D shows detection of CAR T cells by flow cytometry in the second patient of FIG. 17B at C1.T3.D14.

FIG. 18A depicts results of flow cytometry of peripheral blood mononuclear cells (PBMC) that have been depleted of their T cells by sequential CD8+ and CD4+ magnetic bead separation.

FIG. 18B depicts results of flow cytometry of PBMC that have been depleted of their T cells by by sequential CD8+ and CD4+ magnetic bead separation of cells shown in FIG. 18A and labelled with 5 μM FL-PLE.

FIG. 18C depicts results of flow cytometry with the PBMC that have been depleted of their T cells by by sequential CD8+ and CD4+ magnetic bead separation. and labelled with 5 μM FL-PLE FIG. 18B then were frozen and thawed.

FIG. 18D depicts a histogram for data presented in FIG. 18A showing FL-PLE integration.

FIG. 18E depicts a histogram for data presented in FIG. 18B showing FL-PLE integration.

FIG. 18F depicts a histogram for data presented in FIG. 18C showing FL-PLE integration.

FIG. 18G depicts a graph showing side scatter for data presented in FIG. 18A showing FL-PLE integration.

FIG. 18H depicts a graph showing side scatter for data presented in FIG. 18B showing FL-PLE integration.

FIG. 18I depicts a graph showing side scatter for data presented in FIG. 18C showing FL-PLE integration.

FIG. 19A depicts a graph for the number of cells over time that underwent a standard rapid expansion protocol (REP) using irradiated TM-LCL and PBMCs.

FIG. 19B depicts a graph for the number of cells over time that underwent a fluorescein REP (FREP) using irradiated TM-LCL loaded with 5 μM FL-PLE at a 7:1 target to effector ratio was performed.

FIG. 19C depicts a graph for the number of cells over time that underwent a FREP performed using irradiated autologous PBMC (depleted of T cells) loaded with 5 μM FL-PLE at a 7:1 target to effector ratio.

FIG. 19D depicts a graph for the number of cells over time that underwent a FREP performed using irradiated autologous PBMC (depleted if T cells) loaded with 5 μM FL-PLE at a 14:1 target to effector ratio.

FIG. 19E depicts a graph for the number of cells over time that underwent a FREP using frozen, thawed, and irradiated autologous PBMC (depleted T cells) loaded with 5 μM FL-PLE (prior to freeze) at a 7:1 target to effector ratio.

FIG. 20A depicts a graph for flux over time for mice administered anti-FL CAR T cells including average results for groups: (A) administered anti-FL CAR T cells only (circle); (B) also administered 20e6 irradiated TM-LCL (square); (C) also administered 5e6 irradiated TM-LCL loaded with 5 μM FL-PLE (triangle); and (D) also administered 20e6 irradiated TM-LCL loaded with 5 μM FL-PLE (triangle pointing down).

FIG. 20B depicts a graph for flux over time for group (A) mice administered anti-FL CAR T cells only.

FIG. 20C depicts a graph for flux over time for group (B) mice administered anti-FL CAR T cells and 20e6 irradiated TM-LCL.

FIG. 20D depicts a graph for flux over time for group (C) mice administered anti-FL CAR T cells and 5e6 irradiated TM-LCL loaded with 5 μM FL-PLE.

FIG. 20E depicts a graph for flux over time for group (D) mice administered anti-FL CAR T cells and 20e6 irradiated TM-LCL loaded with 5 μM FL-PLE.

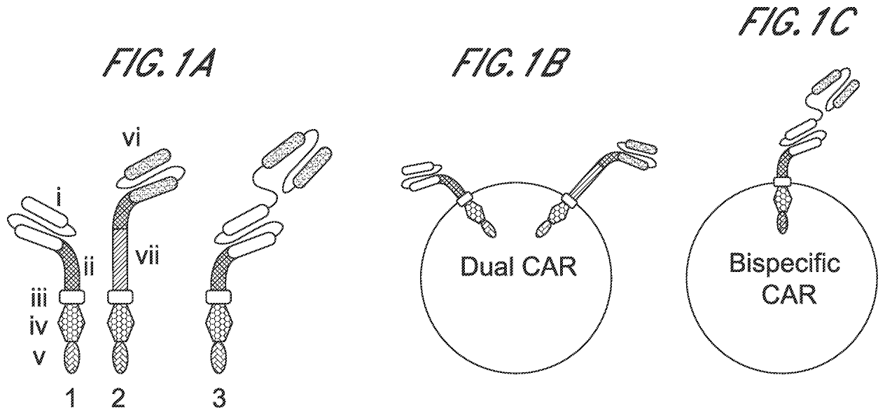

### DEFINITIONS

As used herein, “about” can indicate that a value includes the inherent variation of error for the method being employed to determine a value, or the variation that exists among experiments.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), or fragments generated by any of ligation, scission, endonuclease action, or exonuclease action. Nucleic acid molecules can be composed of monomers that are naturally occurring nucleotides (such as DNA and RNA), or analogs of naturally occurring nucleotides (e.g., enantiomeric forms of naturally occurring nucleotides), or a combination of both. Modified nucleotides can have alterations in sugar moieties or in pyrimidine or purine base moieties. Sugar modifications include, for example, replacement of one or more hydroxyl groups with halogens, alkyl groups, amines, or azido groups, or sugars can be functionalized as ethers or esters. Moreover, the entire sugar moiety can be replaced with sterically and electronically similar structures, such as aza-sugars or carbocyclic sugar analogs. Examples of modifications in a base moiety include alkylated purines or pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate, and the like. The term “nucleic acid molecule” also includes “peptide nucleic acids,” which comprise naturally occurring or modified nucleic acid bases attached to a polyamide backbone. Nucleic acids can be either single stranded or double stranded. “Coding for” is used herein to refer to the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system. A “nucleic acid sequence coding for a polypeptide” includes all nucleotide sequences that are degenerate versions of each other and that code for the same amino acid sequence. “Specific” or “Specificity” can refer to the characteristic of a ligand for the binding partner or alternatively, the binding partner for the ligand, and can include complementary shape, charge and hydrophobic specificity for binding. Specificity for binding can include stereospecificity, regioselectivity or chemoselectivity. In some alternatives, a method of making a nucleic acid encoding a chimeric antigen receptor is provided such that a nucleic acid encoding a chimeric antigen receptor is generated that is specific for a hapten or a tumor antigen.

A “vector” or “construct” is a nucleic acid used to introduce heterologous nucleic acids into a cell that can also have regulatory elements to provide expression of the heterologous nucleic acids in the cell. Vectors include but are not limited to plasmid, minicircles, yeast, or viral genomes. In some alternatives, the vectors are plasmid, minicircles, viral vectors, DNA or mRNA. In some alternatives, the vector is a lentiviral vector or a retroviral vector. In some alternatives, the vector is a lentiviral vector.

“Chimeric antigen receptor” or “CAR” or “Chimeric T cell receptor” have their plain and ordinary meaning when read in light of the specification, and may include but is not limited to, for example, a synthetically designed receptor comprising a ligand binding domain of an antibody or other protein sequence that binds to a molecule associated with the disease or disorder and is linked via a spacer domain to one or more intracellular signaling domains of a T cell or other receptors, such as a costimulatory domain. Chimeric receptors can also be referred to as artificial T cell receptors, chimeric T cell receptors, chimeric immunoreceptors, or chimeric antigen receptors (CARs). These CARs are engineered receptors that can graft an arbitrary specificity onto an immune receptor cell. The term chimeric antigen receptors or “CARs” is also considered by some investigators to include the antibody or antibody fragment, the spacer, signaling domain, and transmembrane region. However, due to the surprising effects of modifying the different components or domains of the CAR described herein, such as the epitope binding region (for example, antibody fragment, scFv, or portion thereof), spacer, transmembrane domain, or signaling domain), the components of the CAR are frequently distinguished throughout this disclosure in terms of independent elements. In some alternatives, the spacer for the chimeric antigen receptor is selected (e.g., for a particular length of amino acids in the spacer) to achieve a desired orientation, avidity, or binding characteristics for the CAR. CARs having varying lengths of spacers, e.g., presented on cells are then screened for the ability to bind or interact with a target moiety to which the CAR is directed. Exemplary target moieties may include, but is not limited to biotin, digoxigenin, dinitrophenol, green fluorescent protein (GFP), yellow fluorescent protein, orange fluorescent protein, red fluorescent protein, far red fluorescent protein, or fluorescein (e.g., Fluorescein isothiocyanate (FITC)). The target moieties to which the CARs bind or interact can be presented on a substrate, such as a membrane, bead, or support (e.g., a well) or a binding agent, such as a lipid (e.g., PLE), hapten or a cell, such as a cell presenting a hapten e.g., a cancer cell associated with the target-bearing hapten. The CAR may also be specific for a hapten on other cells or an antigen present on a cancer cell or pathogen such as, a virus or bacteria. By one approach, the substrate or binding agent comprising the desired target moiety is contacted with a plurality of cells comprising a CAR or TCR specific for said target moiety and the level or amount of binding of the cells comprising the CAR or TCR to the target moiety present on the substrate or binding agent is determined. Such an evaluation of binding may include staining for cells bound to target moieties or evaluation of fluorescence or loss of fluorescence. Again, modifications to the CAR structure, such as varying spacer lengths, can be evaluated in this manner. In some approaches, a cell comprising a hapten is also provided such that the method comprises contacting a cell with a hapten in order to stimulate a T cell with a second CAR or TCR that is specific for a target moiety or antigen on target cell, such as a cancer cell, tumor cell or target virus.

“Specific” or “Specificity” can refer to the characteristic of a ligand for the binding partner or alternatively, the binding partner for the ligand, and can include complementary shape, charge and hydrophobic specificity for binding. Specificity for binding can include stereospecificity, regioselectivity and/or chemoselectivity. In some alternatives, a method of making a nucleic acid encoding a chimeric antigen receptor is provided such that a nucleic acid encoding a chimeric antigen receptor is generated that is specific for a tumor antigen or a hapten.

Antigen” or “Ag” as used herein refers to a molecule that provokes an immune response. This immune response can involve either antibody production, or the activation of specific immunologically-competent cells, or both. It is readily apparent that an antigen can be generated synthesized, produced recombinantly or can be derived from a biological sample. Such a biological sample can include, but is not limited to a tissue sample, a tumor sample, a cell or a biological fluid such, for example, blood, plasma or ascites fluid. “Antitumor effect” as used herein, refers to a biological effect, which can be manifested by a decrease in tumor volume, a decrease in the number of tumor cells, a decrease in the number of metastases, an increase in life expectancy, or a decrease of various physiological symptoms associated with the cancerous condition. An “antitumor effect” can also be manifested by a decrease in recurrence or an increase in the time before recurrence. In some alternatives provided herein the CAR bearing T cells have an antitumor effect.

“Bi-specific chimeric antigen receptor” refers to a CAR that comprises two domains, wherein the first domain is specific for a first ligand, and wherein the second domain is specific for a second ligand. In some alternatives, the first ligand is a hapten. In some alternatives, the second ligand is a tumor-specific ligand. In some alternatives, the bi-specific CAR comprises two scFv domains, wherein the first scFv domain is specific for the tumor specific ligand, and the second scFv domain is specific for a hapten.

“Ligand” as used herein refers to a substance that binds specifically to another substance to form a complex. Examples of ligands include epitopes on antigens, molecules that bind to receptors, substrates, inhibitors, hormones, or activators. “Ligand binding domain” as used herein refers to substance or portion of a substance that binds to a ligand. Examples of ligand binding domains include antigen binding portions of antibodies, extracellular domains of receptors, or active sites of enzymes. “Percent (%) amino acid sequence identity” with respect to the chimeric receptor polypeptide sequences identified herein is defined as the percentage of amino acid residues in a candidate sequence that are identical with the amino acid residues in the reference sequence for each of the ligand binding domain, spacer, transmembrane domain, or the lymphocyte activating domain, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, % amino acid sequence identity values generated using the WU-BLAST-2 computer program [Altschul et al., Methods in Enzymology, 266:460-480 (1996)] uses several search parameters, most of which are set to the default values. Those that are not set to default values (i.e., the adjustable parameters) are set with the following values: overlap span=1, overlap fraction=0.125, word threshold (T)=11 and scoring matrix=BLOSUM62. A % amino acid sequence identity value is determined by dividing (a) the number of matching identical amino acid residues between the each or all of the polypeptide amino acid sequence of the reference chimeric receptor sequence. In some alternatives, a nucleic acid encoding a CAR, of a polypeptide of a CAR can comprise a percent sequence identity to a sequence set forth in TABLE 3 or TABLE 4.

In some embodiments, cells can be engineered for the expression of the two CARs or of a bispecific CAR by a vector, such as a viral vector, such as gammaretrovirus or lentivirus vectors, or a CRISPR/CAS9 system. Such techniques for genetically engineering T cells for CAR or bispecific CAR expression are known to those of skill in the art. In some alternatives, the vector is a transposon, integrase vector system, or an mRNA vector.

“Co-stimulatory domain,” or “intracellular signaling domain” has its plain and ordinary meaning when read in light of the specification, and may include but is not limited to, for example, a signaling moiety that provides to T cells a signal which, in addition to the primary signal provided by for instance the CD3 zeta chain of the TCR/CD3 complex, mediates a T cell response, including, but not limited to, activation, proliferation, differentiation, cytokine secretion, and the like. A co-stimulatory domain can include all or a portion of, but is not limited to, CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83. In some alternatives, the co-stimulatory domain is an intracellular signaling domain that interacts with other intracellular mediators to mediate a cell response including activation, proliferation, differentiation and/or cytokine secretion.

In some alternatives described herein, the CAR is specific for hapten. In some alternatives described herein, a second CAR is present on the T cell that is specific for an antigen on a cell or tumor cell. In some alternatives herein, the CAR comprises a co-stimulatory domain. In some alternatives the co-stimulatory domain is CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83, or a portion thereof.

A “transmembrane domain” is a region of a protein that is hydrophobic that can reside in the bilayer of a cell to anchor a protein that is embedded to the biological membrane. Without being limiting, the topology of the transmembrane domain can be a transmembrane alpha helix. In some alternatives of the method of making genetically modified T-cells, which have a chimeric antigen receptor, the vector comprises a sequence encoding a transmembrane domain. In some alternatives of the method, the transmembrane domain comprises a CD28 transmembrane sequence or a fragment thereof that is a length of 10, 11, 12, 13, 14, 15, 16, 17, 18, 19, 20, 21, 22, 23, 24, 25, 26, 27, or 28 amino acids or a length within a range defined by any two of the aforementioned lengths. In some alternatives of the method, the CD28 transmembrane sequence or fragment thereof comprise 28 amino acids in length. In some alternatives, the chimeric receptor comprises a transmembrane domain. The transmembrane domain provides for anchoring of the chimeric receptor in the membrane.

A “T cell receptor” or “TCR has their plain and ordinary meaning when read in light of the specification, and may include but is not limited to, for example, a molecule that is found on the surface of T lymphocytes or T cells that is responsible for the recognition of fragments of antigen bound to a major histocompatibility complex molecule.

As used herein, “hapten” has its plain and ordinary meaning when read in light of the specification, and may include but is not limited to, for example, a small molecule binding moiety. In some embodiments, a hapten may not induce an immune response, or a significant immune response; however, a hapten attached to a carrier may induce an immune response. In some embodiments a hapten may be tethered to a carrier, such as a cell.

In some embodiments, a hapten can be any Alexa Fluor fluorophore. In some embodiments, a hapten can be any small molecules that elicit an immune response only when attached to a large carrier such as a protein; the carrier may be one that also does not elicit an immune response by itself. In some embodiments, a hapten can be any small molecule which, when combined with a larger carrier such as a protein, can elicit the production of antibodies which bind specifically to it (in the free or combined state). In some embodiments, a hapten can also be peptides, others larger chemicals, and aptamers. In some embodiments, a hapten can by any hapten provided in a hapten database accessible on the World Wide Web.

Non-limiting examples of haptens useful with embodiments provided herein are listed in TABLE 1.

TABLE 1
EXAMPLE HAPTENS USEFUL FOR EMBODIMENTS HEREIN

Alexa Fluor 405; Alexa Fluor 430; Alexa Fluor 500; Alexa Fluor 514; Alexa Fluor 532;
Alexa Fluor 546; Alexa Fluor 555; Alexa Fluor 568; Alexa Fluor 594; Alexa Fluor 610;
Alexa Fluor 633; Alexa Fluor 635; Alexa Fluor 647; Alexa Fluor 660; Alexa Fluor 680;
Alexa Fluor 700; Alexa Fluor 750; Alexa Fluor 790; Cascade Blue; Alexa Fluor 488;
BODIPY; Dansyl chloride; Oregon Green; Lucifer yellow; Rhodamine;
Tetramethylrhodamine; Nitrotyrosine; digoxigenin; 2,4-Dichlorophenoxyacetic acid;
Atrazine (2-Chloro-4-(ethylamino)-6-(isopropylamino)-s-triazine); Nicotine
(3-(1-Methyl-2-pyrrolidyl)pyridine; Black Leaf); Morphine (morph; Morphine Sulfate);
2,4-Dinitrochlorobenzene (1-Chloro-2,4-dinitrobenzene; DNCB;Dinitrochlorobenzene);
4-chloro-6-(ethylamino)-1,3,5-triazine-2-(6-aminohexanecarboxylic acid); Structurally
related s-triazines (Modifications: H/Cl/C6 R1 = NH2— R2 = —Cl
R3 = —NH—(CH2)5—COOH; iPr/Cl/nBu R1 = CH3)2—CH—NH— R2 = —Cl
R3 = —NH—(CH2)3—(CH3)); Ametryn (2-Ethylamino-4-isopropylamino-6-methylthio-
1,3,5-triazine); Deethylatrazine (DEA) (Structurally related s-triazines); Deisopropylatrazine
(DIA) (Structurally related s-triazines); Deethyldeisopropylatrazine (DEDIA)
(Structurally related s-triazines); Deethyldeisopropylatrazine (DEDIA)
(Structurally related s-triazines); HydroxyAtrazine (HA) (Structurally related
s-triazines); DeisopropylHydroxyAtrazine (DIHA) (Structurally related s-triazines);
DeethylDeisopropylHydroxyAtrazine (DEDIHA) (Structurally related s-triazines);
Simazine (Structurally related s-triazines); Desmetryne (Structurally related s-triazines);
Prometryne (Structurally related s-triazines); 2-hydroxyatrazine (atrazine derivative);
2-hydroxypropazine (structurally related s-triazine); 2-hydroxysimazine; N-(4-Amine-
6-hydroxy-[1,3,5]triazin-2-yl)-4-aminobutanoic Acid (Modification: R1 = NH2
R2 = NH(CH2)3COOH R3 = OH); SulcoFuron; 5-chloro-2-{4-chloro-2-[3-(3,4-
dichlorophenyl)ureido]phenoxy}benzenesulfonic acid; FlucoFuron (1,3-bis(4-chloro-
α,α,α-trifluoro-m-tolyl)urea); Agatharesinol; Sequirin C; Sugiresinol;
Hydroxysugiresinol; Hinokiresinol; Coniferyl alcohol; Cinnamyl alcohol; p-Coumaric acid;
Cinnamic acid; p-Coumaric acid; Cinnamic acid; Hinokinin; Guaiacylglycerol- beta-guaiacyl
ether; Morphine-3-glucuronide(M3G); Codeine; Nor-Codeine; 6-Monoacetylmorphine;
(+) Methamphetamine; Ceftazidime; Phenobarbital; p-hydroxyPhenobarbital;
p-aminophenobarbital; Cyclobarbital; 3′-Ketocyclobarbital; 3′-Hydroxycyclobarbital;
Secobarbital; Barbital; Metharbital; Barbituric acid; Thiopental; Thiobarbituric acid;
Primidone; Glutethimide; Pentobarbital; Heroin; Diacetylmorphine; Levallorphan;
L-11-Allyl-1,2,3,9,10,10a-hexahydro-4H-10,4a-iminoethanophenanthren-6-ol;
Pethidine (Demerol; Dolantin; Meperidine; Ethyl 1-methyl-4-phenylpiperidine-4-
carboxylate; Isonipecaine); Methamphetamine; d-Desoxyephedrine; Methedrine;
Tolopropamine; Pratalgin; Pragman. Benzoylecgonine; 3-Carboxymethylmorphine; Cocaine;
5-benzimidazolecarboxylic acid; ABA (4-acetyl benzoic acid); Dexamethasone;
Flumethasone; 6alpha; 9 alpha-difluoro-11 beta,17,21-trihydroxy-16 alpha-methylpregna-
1,4-diene-3,20-dione; 9 alpha-fluoro-11 beta,17,21-trihydroxy-16 beta-methylpregna-1,4-
diene-3,20-dione; 9-alpha-fluroprednisolone; Desoxymethasone; Triamcinolone; 9 alpha-
fluoro-11 beta,16 alpha; 17,21-tetrahydroxypregna-1,4-diene-3,20-dione; Fluocortolone;
6alpha-fluoro-11 beta,21-dihydroxypregna-1,4-diene-3,20-dione; Cortisol; 11 beta,17,21-
trihydroxypregna-4-ene-3,20-dione; Prednisone; 17,21-dihydroxypregn-4-ene-3,11,20-
trione; Methylprednisolone; 11 beta,17,21-trihydroxy-6 alpha-methylpregna-1,4-diene-
3,20-dione; Triamcinolone hexacetonide; 21-(3,3-dimethyl-1-oxobutoxy)-9 alpha-fluoro-
11-hydroxy-16,17-[(1-methylethylidene) bis(oxy)]pregna-1,4-diene-3,20-dione;
Carbofuran; 2,3-dihydro-2,2-dimethyl-7-benzofuranyl methylcarbamate; BFNP (3-[[(2,3-
dihydro-2,2-dimethyl-7-benzofuranyloxy)carbonyl]amino]propanoic acid); Carbofuran
derivative; 2,3-dihydro-2,2-dimethyl-7-benzofuranol; Bendiocarb; Carbaryl; Methiocarb;
Propoxur; Aldicarb; Methomyl; Benalaxyl; methyl N-(phenylacetyl)-N-(2,6-xylyl)-DL-
alaninate; Bn-Ba (4-[2-(N-phenylacetyl-N-2,6-xylylamino)propionamido] butyric acid);
Bn-COOH (4-[2-(N-phenylacetyl-N-2,6-xylyl-DL-alanine); Benalaxyl derivative;
Furalaxyl; Metalaxyl; Acetochlor; Dimetachlor; Metolachlor; 2-chloro-6′-ethyl-N-(2-
methoxy-1-methylethyl)acet-o-toluidide; Diethathyl-ethyl; Benzoylprop-ethyl;
Benzoylprop-ethyl; 2,4,5-Trichlorophenoxyacetic acid; 2-chloro-6′-ethyl-N-(2-methoxy-1-
methylethyl)acet-o-toluidide; Diethathyl-ethyl; Benzoylprop-ethyl; Propachlor; Propachlor;
2,4,5-Trichlorophenoxyacetic acid; 2,4,5,T; Weedone; 2,4-Dichlorophenoxybutyric acid
(2,4-DB); 2,4-DB; Butanoic acid; 4-(2,4-dichlorophenoxy)-; Butoxone; Embutone; MCPA;
2-Methyl-4-chlorophenoxyacetic acid; Metaxon; Dichlorprop (2,4-DP); 1-[(2-
chloro)phenylsulfonyl]monoamidosuccinic acid; Chlor sulfuron; chlorbromuron;
amidosulfuron; chlortoluron; isoproturon; diuron; Linuron O-Methyl-O-(4-nitrophenyl)-N-
(4-carboxybutyl)-phosphoramidothioate Parathion-methyl; O,O-dimethyl O-4-nitrophenyl
phosphorothioate; Methaphos; Wolfatox; Dimethylparathion; Metacide.,Parathion-ethyl;
DIETHYL P-NITROPHENYL THIOPHOSPHATE; O,O-DIETHYL O-(P-NITROPHENYL)
PHOSPHOROTHIOATE;,Fenitrothion; O,O-dimetyl O-4-nitro-m-tolyl phosphorothioate;
Fenthion,O,O-dimethyl O-4-methylthio-m-tolyl phosphorothioate; Bromophos,O-4-
bromo-2,5-dichlorophenyl O,O-dimethyl phosphorothioate; chlorpyrifos-
methyl,O,O-dimethyl O-3,5,6-trichloro-2-pyridyl phosphorothioate; Oxidized parathion-
methyl,Paraoxon; phosphoric acid; O,O-diethyl O-(4-nitrophenyl) ester,Diazinon,O,O-
diethyl O-2-isopropyl-6-methylpyrimidin-4-yl phosphorothioate; Azinphos-methyl;
pirimiphos-methyl; O-2-diethylamino-6-methylpyrimidin-4-yl O,O-dimethyl
phosphorothioate; Methidathion; S-2,3-dihydro-5-methoxy-2-oxo-1,3,4-thiadiazol-3-
ylmethyl O,O-dimethyl phosphorodithioate; Dimethylchlororothiophosphate;
4-NITROPHENOL; p-nitrophenol; Phenolic derivative (Modification On benzene ring;
R1 = OH R2 = NO2 R3 = H R4 = CH2COOH R5 = H R6 = H); 2-Nitrophenol;
o-Nitrophenol; 3-Nitrophenol; m-nitrophenol; 2,4-Dinitrophenol; 3,4-Dinitrophenol;
2,5-Dinitrophenol; 2,4-Dinitro-6-methylphenol; 2,3,6-trinitrophenol; 2-Chlorophenol;
4-Chloro-3-methylphenol,Fenitroxon; 3-Methyl-4-nitrophenol; Nonylphenol,HOM(3-
[2-hydroxy-5nitro benzylthio] propionic acid; Phenol,Delor 103; Polyclorinated Biphenyls;
Delor 104; Polyclorinated Biphenyls; Delor 105,Polyclorinated Biphenyls,Delor
106; 4,4′-Dichlorobiphenyl,PCB congeners; 2,4,4′-Trichlorobiphenyl; PCB congeners,2,4′-;
PCB congeners; 2,2′-Dichlorobiphenyl,PCB congeners; 2,4,5-Trichlorobiphenyl,PCB
congeners; 3,3′,4,4′-Tetrachlorobiphenyl,PCB congeners; PCB congeners; 2,2′,4,4′,5,5′-
Hexachlorobiphenyl; 2-(5-Carboxypentanoylamino)-4,4′-dichlorobiphenyl,Biphenyl
derivative,4-chlorophenoxyacetic acid,2-Chlorophenoxyacetic acid; DDT,1,1,1-trichloro-2;
2-bis-(p-chlorophenyl)ethane,DDE,1,1-dichloro-2; 2-bis(p-chlorophenyl)ethylene,
p-Chlorophenol; 4-Chlorophenol; m-Chlorophenol 3,4-Dichlorophenol; 3,5-Dichlorophenol;
2,3,4-Trichlorophenol; 2,3,5-Trichlorophenol; 3-methylindole; 3-methylindole Derivatives;
4-(3-methylindol-5-yloxy)butanoic acid; 4-(3-methylindol-5-yloxy)butanoic acid;
3-methylindole Derivatives; 6-[n-3-methylindol-5-yloxy carbonyl)amino]hexanoic acid;
6-[n-3-methylindol-5-yloxy carbonyl)amino]hexanoic acid; 3-methylindole Derivatives;
2-[4-(3-methylindol-6-yl)but-1-ylthro]acetic acid; 2-[4-(3-methylindol-6-yl)but-1-
ylthro]acetic acid; 3-methylindole Derivatives; 4-(3-methylindol-6-yl-4-oxo)butanoic acid;
4-(3-methylindol-6-yl-4-oxo)butanoic acid; 3-methylindole Derivatives; 6-(3- methylindol-
7-yloxy)hexanoic acid; 6-(3-methylindol-7-yloxy)hexanoic acid; Indole; Indole-3-Carboxylic
acid; Indole Derivative -Indole-3-Acetic acid; Indole-3-Acetic acid; Indole Derivative -
Indole-3-Propionic acid; Indole-3-Propionic acid; Indole Derivative-Indole-
3-Carbinol,Indole-3-Carbinol; Tryptophan; Tryptamine; 5-Methoxyindole-
3-carboxaldehyde,5-Methoxytryptamine; 5-Methoxyindole; 6-Methoxyindole;
7-Methoxyindole,EB1089(Seocalcitol); EB1089(Seocalcitol) Derivative;
(22E,24E)-Des-A,B-24-homo-26,27-dimethyl-8-[(E)-N-(2-carboxyethyl)-
carbamoylmethylidene]-cholesta-22,24-dien-25-ol; 1 alpha-25-dihydroxyvitamin D3;
25(OH)D3,25-hydroxyvitamin D3,24R,25(OH)2D3; 24R,25-dihydroxyvitamin D3;
Vitamin D2,ergocalciferol; Vitamin D3; cholecalciferol; EB1446; EB1436; EB1445;
EB1470; DeethylHydroxyAtrazine (DEHA) (Structurally related s-triazines); Irgarol 1051;
Flourescein Isothiocyanate; FITC,Metanephrine,NorMetanephrine; Propazine;
Terbutylazine; Terbuthylazine; 6-chloro-N-(1,1-dimethylethyl)-N′-ethyl-1,3,5-triazine-
2,4-diamine; (Structurally related s-triazines); Ametryn (2-Ethylamino-4-isopropylamino-6-methylthio-
1,3,5-triazine (Modification iPr/SCH3/Et R1 = (CH3)2—CH—NH— R2 = —SCH3
R3 = —NH—CH2—CH3; Irgarol; Cyanazine (Modification R1 = Cl R2 = NHCH2CH3
R3 = NHCCN(CH3)2); OH-Terbutylazine; Terbutylazine-2OH; Hydroxytriazine
(EQ-0027); Deisopropylatrazine (Structurally related S-triazine); Desethylterbutylazine
(Structurally related S-triazine); Desethyl-deisopropylatrazine (Structurally related
S-triazine); Atraton; Terbutryn (Structurally related s-triazines); Atrazine derivative
(Modification R1 = — NHCH(CH3)2 R2 = —S(CH2)2COOH R3 = —NHC2H5);
Cyanuric chloride; Trifluralin; (Structurally related s-triazines) tBu/C4/SCH3
(Modification R1 = —NH—C—(CH3)3 R2 = — NH(CH2)3COOH R3 = —SCH3);
Sulphamethazine; (Structurally related s-triazines) 6-[[[4-Chloro-6-(methylamino)]-1,3,5-
triazin-2-yl]amino]hexanoic Acid (Modification Me/Cl/C6 R1 = —NHCH3 R2 = —Cl
R3 = —NH(CH2)5COOH); (Structurally related s-triazines) Procyazine (Modification
R1 = —Cl R2 = —NHcyclopropyl R3 = —NHCCN(CH3)2); (Structurally related
s-triazines); Prometon (Modification R1 = —OCH3 R2 = —NHCH(CH3)2
R3 = —NHCH(CH3)2); (Structurally related s-triazines) Atrazine Mercapturic Acid (AM)
(Modification R1 = —SCH2CH(NHAc)COOH R2 = —NHCH2CH3 R3 = —NHCH(CH3)2);
(Structurally related s-triazines),desethyl atrazine mercapturic acid (desethyl AM)
(Modification R1 = —NAcCys R2 = —NH2 R3 = —NHCH(CH3)2); (Structurally
related s-triazines); deisopropyl atrazine mercapturic acid (deisopropyl AM)
(Modification R1 = —NAcCys R2 = —NHCH2CH3 R3 = —NH2); (Structurally related
s-triazines); didealkylated atrazine mercapturic acid (didealkylated AM) (Modification
R1 = —NAcCys R2 = —NH2 R3 = —NH2); (Structurally related s-triazines); simazine
mercapturate (Modification R1 = —NAcCys R2 = —NHCH2CH3 R3 = —NHCH2CH3);
(Structurally related s-triazines) (Modification R1 = —S(CH2)2COOH
R2 = —NHCH2CH3 R3 = —NHCH2CH3); (Structurally related s-triazines)
(Modification R1 = —Cl R2 = —NHCH(CH3)2 R3 = —NH(CH2)2COOH);
(Structurally related s-triazines) (Modification R1 = —Cl R2 = —NHCH2CH3
R3 = —NH(CH2)2COOH); (Structurally related s-triazines); atrazine mercapturic
acid methyl ester (AM methyl ester) (Modification R1 = —NAcCysME R2 = —NHCH2CH3
R3 = —NHCH(CH3)2); N-acetylcysteine; S-benzyl mercapturate; (Structurally
related s-triazines); simetryn (Modification R1 = —SCH3 R2 = —NHCH2CH3
R3 = —NHCH2CH3); Metribuzin; 4-amino-6-tert-butyl-4,5-dihydro-3-methylthio-1,2,4-triazin-5-one;
Sulpha Drugs; N4-acetyl-sulphamethazine (Modification N4-acetyl-sulphamethazine); Sulpha Drugs;
Sulphathiazole; Sulphathiazole; Sulphamerazine; Sulphamerazine; Sulphaquinoxaline;
Sulphaquinoxaline Sulphachlorpyridazine; Sulphachlorpyridazine; Sulphapyridine;
Sulphadimethoxine; Sulphadimethoxine; Sulphamethoxazole; Sulphamethoxazole;
Sulphisoxazole; Sulphisoxazole; Sulphamethizole; Sulphamethizole; Sulphanilamide;
Sulphanilamide; Sulphaguanidine; Sulphaguanidine; Sulphadiazine; Sulphadiazine;
Sulphamethoxypyridiazine; Sulphamethoxypyridiazine; Pentachlorophenoxipropionic acid;
Pentachlorophenol; PCP; 2,3,5,6-Tetrachlorophenol; 1,2,4,5 Tetrachlorobenzene; 2,4,6
Trichlorophenol; 2-Methoxy-3,5,6-trichloropyridine; 1,3,5 Trichlorobenzene;
1,3 Dichlorobenzene; 2,4,5-Trichlorophenol; 2,6-Dichlorophenol; 3,5,6-Trichloro-2-
pyridinoxiacetic acid; 3,5,6-Trichloro-2-Pyridinol; TCP; 2,4-Dichlorophenol;
2,5-Dichlorophenol; DNC; 4,4′-dinitrocarbanilide; (Structurally related s-triazines);
Dichloroatrazine; (Structurally related s-triazines); Dichlorosimazine; 1-((6-chloropyridin-
3-yl)methyl)imidazolidin-2-imin; Pyridine Derivative; 6-chloropyridine-3-carboxylic acid;
Nicotinic acid; Pyridine Derivative; N-((6-chloropyridin-3-yl)methyl)-N-methylacetamide;
(6-chloropyridin-3-yl)-N-methylmethanamine; (6-chloropyridin-3-yl)methanol;
Imidacloprid; 1-(6-chloro-3-pyridylmethyl)-N-nitroimidazolidin-2-ylideneamine;
Acetamiprid; (E)-N1-[(6-chloro-3-pyridyl)methyl]-N2-cyano-N1-methylacetamidine;
Nitenpyram; Deltamethrin; 1(R)-cis-alpha(S)-3-(2,2-dibromoethenyl)-2,2-
dimethylcyclopropane carboxylic acid cyano(3-phenoxyphenyl)methyl ester; DON;
deoxynivalenol; DON derivative; 15-AcDON (15-acetyldeoxynivalenol); DON
derivative; -AcDON (3-acetyldeoxynivalenol); DON derivative; 3,15-DiacDON (3,15-
diacetyldeoxynivalenol); DON derivative; 3,7,15-TriacDON (3,7,15- Triacetyldeoxynivalenol);
NIV (nivalenol); nivalenol; NIV Derivative; 4-AcNIV (fusarenon X); Flutolanil;
alpha,alpha,alpha-trifluoro-3′-isopropoxy-o-toluanilide; Mepronil; Mebenil;
Benodanil; 24,25(OH)2D3; (24R)-24,25-dihydroxyvitamin D3; 24S,25(OH)2D3; 24S,25-
dihydroxyvitamin D3; 25R,26(OH)2D3; 25R,26-dihydroxyvitamin D3; 25S,26(OH)2D3;
25S,26-dihydroxyvitamin D3; 1,24,25(OH)3D3; 1,24,25-trihydroxyvitamin D3; 1,25-
lactone; (23S,25R)-1,25(OH)2 D3 26,23-lactone; 24,25(OH)2--7-DHC; 24,25(OH)2--7-
dehydrocholesterol; 25(OH)D3 3S; 25(OH)D3 3-sulfate; 24,25(OH)2D3 - Hemiglutarate
Derivative; 11 alpha-hemiglutaryloxy-(24R)-24,25-dihydroxyvitamin D3; 24,25(OH)2D3 -
Hemiglutarate Derivative; (24R)-24,25-dihydroxyvitaminD3 -3-hemiglutarate;
24R,25(OH)2D2; 24S,25(OH)2D2; 25(OH)D2; 1,24(OH)2D3; 2,3,6-Trichlorophenol;
Tetrachlorohydroquinone; Pentachloroaniline; Pentachlorobenzene; 2,3-Dinitrotoluene;,
4-Dinitrotoluene; 2,4,5-Trichloronitrobenzene; 3-(3-Hydroxy-2,4,6-trichlorophenyl)-
propanoic acid; 2,3,4,6-Tetrachlorophenol; 2,4,6-Trichloroanisol; 2,4,6-TCA; Pentabromophenol;
PBP; 2,4,6-Tribromophenol; 2,4,6-TBP; 2-Bromo-4-Chlorophenol; 2-B-4-CP
2,4-Dibromophenol; 2,4-DBP; 2,6-Dibromophenol; 2,6-DBP; 4-Bromophenol; 4-BP;
Furosemide; Ampicillin; Amoxicillin; 6-amino-penicillanic acid (6-APA); Azlocillin;
Bacampicillin; Carbenicillin; Epicillin; Cloxacillin; Dicloxacillin; Metampicillin;
Methicillin; Moxalactam; Oxacillin; Penicillin G; benzyl penicillin; Penicillin V; phenoxy
methyl penicillin; Pheneticillin; Piperacillin; Ticarcillin; Ampicillin hydrolyzed; Penicillin
G hydrolyzed; 3-phenoxybenzoic acid (3-PBAc) Chlorpyrifos; Chlorpyrifos derivatives;
HClo1; Synthesized directly from chlorpyrifos technical grade by substitution of the
chlorine in position 6 by a 3-mercaptopropanoic acid spacer arm; Chlorpyrifos derivatives;
HTCP (Modification HTCP of TCP metabolite was prepared from HClo1 by hydrolysis of
the thiphosphate ester); Zeatin Riboside (trans isomer); Zeatin (trans isomer); N6-(2-
isopentenyl)-adenosine; IPA; N6-(2-isopentenyl)-adenine; 2-iP; Benzyladenine; Kinetin;
monuron; monolinuron; fenuron; neburon; propanil; propham; chloropropham;
4-chloroaniline; Methyl Urea Derivative; 1-(3-Carboxypropyl)-3-(4-chlorophenyl)-1-
methylurea; Methyl Urea Derivative; 1-(5-Carboxypentyl)-3-(4-chlorophenyl)-1-
methylurea; metabromuron; Sennoside B; SB; Sennoside B possessed a erythro
configuration between C-10 and C-10′; Sennoside A (Modification Sennoside A possessed
a threo configuration between C-10 and C-10); Rhein; Emodin; Aloe-emodin; Barbaloin;
1,4 Dihydroxyanthraquinone; Rhaponticin; Galic acid; Vanillic acid; Caffeic acid;
Homogentisic acid; Esculin; Cinnamtannin B1; Baicalin; Naringin hydrate; Wogonine;
Wogonine 7-o-beta-glucuronide; Curcumin; delta1-Tetrahydrocannabinolic acid; delta1-
Tetrahydrocannabinol; (+−)-cis-4-Aminopermethrin; 3-(4-Aminophenoxy)benzyl(+−)-cis-3-
(2,2-dichloroethenyl)-2,2-dimethylcyclopropanecarboxylate; Permethrin; trans-Permethrin;
cis-Permethrin; Cypermethrin; Phenothrin; Resmethrin; Cyfluthrin; trans-Permethrin acid
Esfenvalerate; Fluvalinate; Fenpropathrin; cis-permethrin acid; 4-Phenoxybenzoyl alcohol;
Diuron Derivative; 1-(3-Carboxypropyl)-3-(3,4-dichlorophenyl)-1-methylurea; Siduron;
Terbuthiuron; Barban; acid trifluralin; 2,6-dinitro-N--propyl-N-(2-carboxyethyl)-4-
(trifluoromethyl)benzenamine; TR-13; 2-ethyl-7-nitro-1-propyl-5-(trifluoromethyl)-1H-
benzimidazole; benefin; 2,6-dinitro-N-butyl-N-ethyl-4-(trifluoromethyl)benzenamine;
TR-2; 2,6-dinitro-N-propyl-4-(trifluoromethyl)benzenamine; ethalfluaralin; 2,6-dinitro-N-
ethyl-N-(2-methyl-2-propenyl)-4-(trifluoromethyl)benzenamine; TR-40; N-(2,6-dinitro-4-
(trifluoromethyl)phenyl)-N-propylpropanamide; TR-15; 2-ethyl-4-nitro-6-(trifluoromethyl)-
1H-benzimidazole; TR-3; 2,6-dinitro-4-(trifluoromethyl)benzenamine; TR-6;
3-nitro-5-(trifluoromethyl)-1,2-benzenediamine; TR-9; 5-(trifluoromethyl)-1,2,3-
benzenetriamine; TR-21; 4-(dipropylamino)-3,5-dinitrobenzoic acid; TR-36M; 3-methoxy-
2,6-dinitro-N,N-dipropyl-4-(trifluoromethyl)benzinamine; oryzalin; 3,5-dinitro-4-
(dipropylamino)benzenesulfonamide; pendimethalin; 2,6-dinitro-N-(1-ethylpropyl)-3,4-
dimethylbenzenamine; penta galloyl glucose; Pyrene Pyrene-1-carboxaldehyde;
Phenanthrene; Benzo(a)pyrene; 3,4-Benzopyrene; Anthracene; 3,4-Benzopyrene;
Acenapthene; Fluorene; Chrysene; 1,2-Benzphenanthrene; Benzo[g,h,i]perylene;
Benzo[e]pyrene; Acenaphthylene; Fluoranthene; Benzo(j,k)fluorene; Indeno-1,2,3-cd-
pyrene; 1,10-(1,2-Phenylene)pyrene; Benzo[a]anthracene; 1,2-Benzanthracene;
Benzo(k)fluoranthene; Naphthalene; Benzo[a]fluoranthene; Dibenzo[ah]anthracene;
1,2:5,6-Dibenzanthracene; 2,3-Diaminonaphthalene; 2,6-Dinitroaniline; 17-beta-estradiol
(ED); estra-1,3,5(10)-triene-3,17-beta-diol; Trifluralin derivative; 2,6-dinitro-4-tri-
fluoromethylaniline; Trifluralin derivative; N-(2,6-dinitro-4-trifluoromethylphenyl)-6-
aminohexanoic acid; Trifluralin derivative; N-(2,6-dinitro-4-trifluoromethylphenyl)-N-
methyl-6-aminohexanoic acid; Trifluralin derivative; N-(2,6-dinitro-4-trifluoromethylphenyl)-
N-propyl-6-aminohexanoic acid; Trifluralin derivative; N-(2,6-dinitro-4-
trifluoromethylphenyl)-6-aminohexanoic acid methyl ester; Trifluralin derivative; N-(2,6-
dinitro-4-trifluoromethylphenyl)-6-aminohexanoic acid tert-butyl ester; Benfluralin;
Ethalfluralin; Trifluralin derivative; 2,6-Dinitro-4-trifluoromethylphenol; Isopropalin;
Aniline; 2-Hydroxybenzotrifluoride; N-propyl-6-aminohexanoic acid; N-methyl-6-
aminohexanoic acid; MHPG Derivatives; D-MHPG (D-3-methoxy-4-hydroxyphenylglycol);
MHPG Derivatives; L-MHPG (L-3-methoxy-4-hydroxyphenylglycol); MHPG Derivatives;
DL-MHPG (DL-3-methoxy-4-hydroxyphenylglycol); Isomeric mixture of D-MHPG and
L-MHPG forms; MHPG Derivatives; DL-MHPG-SO4 (DL-3-methoxy-4-hydroxyphenylglycol-
sulfate) Modification can include Isomeric mixture of D-MHPG-SO4 and L-MHPG-SO4
forms; Serotonin; 5-HT; 5-hydroxydopamine (5-4HDA); 3,4-dihydroxyphenylglycol
(DOPEG); Dopamine; 4-(2-aminoethyl)pyrocatechol; 3-hydroxytyramine;
3,4-dihydroxyphenethylamine; L-3,4-dihydroxyphenylalanine; L-DOPA;
Vanillomandelic acid; DL-VMA; Homovanillic acid; Norepinephrine; DL-NE;
D-Epinephrine; D-E; 3-methoxythyramine; MTA; 3-methoxytyrosine; MTyr;
3,4-dihydroxymandelic acid; DL-DOMA; 3,4-dihydroxyphenyl acetic acid; DOPAC;
L-Phenylalanine; Tyramine; p-tyramine; 4-(2-Aminoethyl)phenol; D-Mandelic acid;
Homocatechol; Octopamine; DL-Octopamine; Azinphos-Ethyl; S-(3,4-dihydro-4-
oxobenzo[d]-[1,2,3]-triazin-3-ylmethyl) O,O-diethyl phosphorodithioate; Phosmet;
O,O-dimethyl S-phthalimidomethyl phosphorodithioate; Folpet;
N-[(Trichloromethyl)thio]phthalimide; Tetramethrin; (1-Cyclohexene-1,2-
dicarboximido)methyl-2,2-dimethyl-3-(2-methylpropenyl)-cyclopropanecarboxylate;
N-(bromomethyl)phthalimide; N-(Chloromethyl)benzazimide;
6-(N-phthalimidoylmethylthio)hexanoic acid(MFH); Bromacil; 5-bromo-3-sec-butyl-6-
methyluracil; Bromacil Derivative; 5-bromo-6-(hydroxymethyl)-3-(1-methylpropyl)-
2,4(1H,3H)-pyrimidineone; Bromacil Derivative; 5-bromo-3-(2-methylpropyl-6-methyl-
2,4(1H,3H)-pyrimidinedione; Metabolite of Bromacil; Bromacil Derivative; 3-hydroxy-1-
methylpropyl-6-methyl-2,4(1H,3H)-pyrimidinedione (Modification Bromacil Metabolite);
Bromacil Derivative; 6-methyl-3-(1-methylpropyl)-2,4(1H,3H)-pyrimidinedione
(Modification Bromacil Metabolite); Terbacil Derivative; [5-chloro-3-(1,1-dimethylethyl)-
6-(hydroxymethyl)-2,4(1H,3H)-pyrimidinedione; Terbacil; 3-tert-butyl-5-chloro-6-
methyluracil; Bromacil Derivative; Ethyl-5-(5-Bromo-6-methyl-3-(1-methylpropyl)-
2,4(1H,3H)-pyrimidinedione-1-yl)hexanoate; Bromacil Derivative alkylated at N-1;
Bromacil Derivative 5-(5-Bromo-6-methyl-3-(1-methylpropyl)-2,4(1H,3H)-
pyrimidinedione-1-yl)hexanoic Acid (Modification Bromacil Derivative alkylated at N-1);
Bromacil Derivative; -Bromo-6-(Bromomethyl-3-(1-methylpropyl)-2,4(1H,3H)-
pyrimidinedione (Modification Bromacil Derivative substituted at the 6-methyl position);
Bromacil Derivative -[5-Bromo-3-(1-methylpropyl)-2,4(1H,3H)-pyrimidinedione-6-yl]-2-
carboxylpropanoic Acid (Modification Bromacil Derivative substituted at the 6-methyl
position); 3-[5-Bromo-3-(1-methylpropyl)-2,4(1H,3H)-pyrimidinedione-6-yl]propanoic
Acid (Modification Bromacil Derivative substituted at the 6-methyl position); Bromacil
Derivative 5-Bromo-1,6-dimethyl-3-(1-methylpropyl)-2,4(1H,3H)-pyrimidinedione;
Bromacil Derivative 5-Bromo-1-butyl-6-methyl-3-(1-methylpropyl)-2,4(1H,3H)-
pyrimidinedione; Butachlor; N-butoxymethyl-2-chloro-2′,6′-diethylacetanilide; Amidochlor;
N-[(acetylamino)methyl]-2-chloro-N-(2,6-diethylpenyl)acetamide; Nicarbazin;
N,N′-bis(4-nitrophenyl)-compound with 4,6-dimethyl-2(1H)-pyrimidinone (Modification
(DNC + HDP)); 2-hydroxy-4,6-dimethylpyrimidine; HDP; Imazalil; [1-(beta-allyloxy-
2,4-dichlorophenethyl)imidazole]; Imazalil Derivative; EIT- 0073 (Modification Have
a —O(CH2)5—COOH group instead of original —OCH2CH═CH2 group of imazalil);
Penconazole; (RS)-1-(2,4-dichloro-β-propylphenethyl)-1H-1,2,4-triazole; Hexaconazole;
(RS)-2-(2,4-dichlorophenyl)-1-(1H-1,2,4-triazol-1-yl)hexan-2-ol; Propiconazole;
cis-trans-1-[2-(2,4-dichlorophenyl)-4-propyl-1,3-dioxolan-2-ylmethyl]-1H-
1,2,4-triazole; Diclobutrazol; 2RS,3RS)-1-(2,4-dichlorophenyl)-4,4-dimethyl-2-(1H-1,2,4-
triazol-1-yl)pentan-3-ol; Triflumizole; (E)-4-chloro-α,α,α-trifluoro-N-(1-imidazol-1-yl-2-
propoxyethylidene)-o-toluidine; Imazalil Derivative; EIT-0183; Imazalil Derivative; EIT-
0180; Imazalil Derivative; EIT-0111; Imazalil Derivative; EIT-0158; Imazalil Derivative;
K-240; Chlorothalonil; tetrachloroisophthalonitrile Modification On benzene Ring R1 = CN
R2 = Cl R3 = CN R4 = Cl R5 = Cl R6 = Cl); Chlorothalonil Derivative-2,4,5,6-tetrachloro-
3-cyanobenzamide (Modification On benzene Ring R1 = CONH2 R2 = Cl R3 = CN R4 = Cl
R5 = Cl R6 = Cl); Chlorothalonil Derivative-2,5,6- trichloro-4-hydroxyisophthalonitrile
(Modification On benzene Ring R1 = CN R2 = Cl R3 = CN R4 = OH R5 = Cl R6 = Cl);
3-carbamyl-2,4,5-trichlorobenzoic acid (Modification On benzene Ring R1 = CONH2
R2 = Cl R3 = COOH R4 = H R5 = Cl R6 = Cl); Pentachloronitrobenzene (Modification On
benzene Ring R1 = NO2 R2 = Cl R3 = Cl R4 = Cl R5 = Cl R6 = Cl); Benzene hexachloride;
Hexachlorobenzene; BHC; Lindane (Modification On benzene Ring R1 = Cl R2 = Cl
R3 = Cl R4 = Cl R5 = Cl R6 = Cl); 2,4,5,6-tetrachlorophenol (Modification On benzene
Ring R1 = OH R2 = Cl R3 = H R4 = Cl R5 = Cl R6 = Cl); Carbaryl Derivative;
Ethylcarbamate (Modification R1 = OCONHCH2CH3 R3 = H); 1-Naphthol;
1-naphthaleneacetamide; -(1-naphthyl)acetamide; Carbaryl Derivative; 1-Methylcarbonate
(Modification R1 = OCOOCH3 R2 = H; Carbaryl Derivative; 1-Ethylcarbonate
(Modification R1 = OCOOCH2CH3 R2 = H); Carbaryl Derivative 2-Ethylcarbonate
(Modification R1 = H R2 = OCOOCH2CH3; Carbaryl Derivative; 1-Ethylthiocarbonate
(Modification R1 = OCOSCH2CH3 R2 = H); Carbaryl Derivative; 2-Ethylthiocarbonate
(Modification R1 = H R2 = OCOSCH2CH3); Naptalam; N-1-naphthylphthalamic acid;
Carbaryl Derivative; 3-hydroxycarbaryl(Modification R1 = OCONHCH3 R2 = H R3 = OH
R4 = H R5 = H); Carbaryl Derivative 4-hydroxycarbaryl (Modification R1 = OCONHCH3
R2 = H R3 = H R4 = OH R5 = H); Carbaryl Derivative 5-hydroxycarbaryl (Modification
R1 = OCONHCH3 R2 = H R3 = H R4 = H R5 = OH); Carbaryl Derivative;
1-(5-Carboxypentyl)-3-(1-naphthyl)urea (Modification R1 = NHCONH(CH2)5COOH
R2 = H); (Structurally related s-triazines) -Aziprotryn; 4-azido-N-isopropyl-6-methylthio-
1,3,5-triazin-2-ylamine (Modification R1 = —SCH3 R2 = —N3 R3 = —CH(CH3)2);
(Structurally related s-triazines); 2-(ethylamino)-4-(methylthio)-6-aminotriazine
(Modification R1 = —SCH3 R2 = —NH—C2H5 R3 = —NH2); (Structurally related
s-triazines) - 2-amino-4-(methylthio)-6-(isopropylamino)triazine (Modification R1 = —SCH3
R2 = —NH2 R3 = —NH—CH(CH3)2); (Structurally related s-triazines) - 2-
amino-4-methoxy-6-(isopropylamino)triazine (Modification R1 = —OCH3 R2 = —NH2
R3 = —NH—CH(CH3)2); TCP Derivative (3,5,6- trichloro-2-pyridinol
Derivative); 3-(3,5-dichloro-6-hydroxy-2-pyridyl)thiopropanoic Acid; p-nitrosuccinanilic
acid (PNA-S); PNA-S; PNA-C; p-nitro-cis-1,2-cyclohexanedicarboxanilic
acid; Nitroaniline Derivative; 2-nitroaniline; o-Nitroaniline; Nitroaniline Derivative- 3-
nitroaniline; m-Nitroaniline; Nitroaniline Derivative - 4-nitroaniline; p-Nitroaniline;
Aeromatic Alcohols; 4-nitrobenzyl alcohol; Aeromatic Alcohols - 4-nitrophenethyl
alcohol; Aeromatic Alcohols 2-nitrobenzyl alcohol; Aeromatic Alcohols; 3-nitrobenzyl
alcohol; Urea Derivative-1-benzyl-3-(4-nitrophenyl)urea; Urea Derivative-
1-(3-chlorophenyl)-3-(2-methoxy-5-nitrophenyl)urea; Urea Derivative - 1-(3-
chlorophenyl)-3-(4-methoxy-3-nitrophenyl)urea; Urea Derivative - 1-(4-chlorophenyl)-3-
(4-nitrophenyl)urea; Urea Derivative -(2-fluorophenyl)-3-(2-mehtoxy-4-nitrophenyl)urea;
1-(3-mehtoxyphenyl)-3-(3-nitrophenyl)urea; Carbofuran Derivative m Carbofuran-phenol;
Carbofuran-hydroxy; Carbofuran-keto; Carbosulfan; ,3-dihydro-2,2-dimethylbenzofuran-7-yl
(dibutylaminothio)methylcarbamate; Benfuracarb; N-[2,3-dihydro-2,2- dimethylbenzofuran-
7-yloxycarbonyl(methyl)aminothio]-N-isopropyl-β-alininate; Furathiocarb;
2,3-dihydro-2,2-dimethyl-7-benzofuranyl 2,4-dimethyl-5-oxo-6-oxa-3-thia-
2,4-diazadecanoate; Carbofuran Derivative; 4-[[(2,3-Dihydro-2,2-dimethyl-7-
benzofuranyloxy)carbonyl]-amino]butanoic Acid (BFNB) (Modification n = 3 X = CH2);
Endrin; nendrin; (1R,4S,4aS,5S,6S,7R,8R,8aR)-1,2,3,4,10,10-hexachloro-1,4,4a,5,6,7,8,8a-
octahydro-6,7-epoxy-1,4:5,8-dimethanonaphthalene; Heptachlor; 1,4,5,6,7,8,8-heptachloro-
3a,4,7,7a-tetrahydro-4,7-; Chlordane; 1,2,4,5,6,7,8,8-octachloro-2,3,3a,4,7,7a-hexahydro-
4,7-methanoindene; Endosulfan (Modification isomer mix of alpha and beta forms);
Endosulfan (Modification alpha isomeric form); Endosulfan (Modification beta
isomeric form); Endosulfan Derivative; Endosulfan sulfate (Modification sulfate
form); Endosulfan Derivative; Endosulfan diol; Diol metabolite of endosulfan;
Endosulfan Derivative; Endosulfan ether (Modification ether metabolite of endosulfan);
Endosulfan Derivative; hydroxy ether; hydroxy ether metabolite of endosulfan;
Endosulfan Derivative; Endosulfan lactone (Modification lactone metabolite of endosulfan);
Aldrin; Dieldrin; Fenvalerate isomers Modification 1S,2R isomer R:Ph); Fenvalerate
isomers (Modification 1R,2S isomer R:Ph); Fenvalerate isomers (Modification 1R,2R
isomer R:Ph); Fenvalerate isomers (Modification 1S,2R/S isomer R:Ph); Fenvalerate
isomers (Modification 1R,2R/S isomer R:Ph); Fenvalerate isomers; fenvalerate
(Modification 1R/S,2R/S isomer R:Ph); Thiabendazole; 2-(thiazol-4-yl)benzimidazole;
Thiabendazole Derivative; 5-hydroxy thiabendazole (Modification 5-OH-TBZ);
Thiabendazole Derivative; 5-NH2-TBZ; Thiabendazole Derivative; methyl benzimidazole
carbamate; Albendazole; Mebendazole; Fenbendazole; Thiabendazole Derivative;
2-succinamido thiabendazole; Thiabendazole Derivative; 2-succinamidothiabendazole;
Cambendazole; Fenvalerate Haptens; Cyano[3-(4-aminophenoxy)phenyl]methyl (S)-4-
Chloro-alpha-(1-methylethyl)benzeneacetate (4-Aminoesfenvalerate); Fenvalerate Haptens;
Benzyl 4-[3-[Cyano[(S)-2-(4-chlorophenyl)-3-methyl-1-
oxobutanoxy]methyl]]phenoxy]benzenepropanoate; Fenvalerate Haptens; Benzyl
3-[Cyano[(S)-2-(4-chlorophenyl)-3-methyl-1-oxobutanoxy]methyl]]phenoxyacetate;
Fenvalerate Haptens; 3-[Cyano[(S)-2-(4-chlorophenyl)-3-methyl-1-
oxobutanoxy]methyl]]phenoxyacetic Acid; Fenvalerate Haptens; Benzyl 6-[3-[Cyano[(S)-
2-(4-chlorophenyl)-3-methyl-1-oxobutanoxy]methyl]]phenoxy]hexanoate; Fenvalerate
Haptens; 6-[3-[Cyano[(S)-2-(4-chlorophenyl)-3-methyl-1-
oxobutanoxy]methyl]]phenoxy]hexanoic Acid Fenvalerate Haptens; 4-[3-[Cyano[(S)-2-(4-
chlorophenyl)-3-methyl-1-oxobutanoxy]methyl]]phenoxy]benzenepropanoic Acid;
(S)-fenvalerate Acid; (Structurally related s-triazines); atrazine mercapturate Modification
R1 = —SCH2CH(NHCOCH3)COOH R2 = —NHCH2CH3 R3 = —NHCH(CH3)2;
Fenthion Hapten; -Methyl O-[3-methyl-4-(methylthio)phenyl] N-(3-
carboxypropyll)phosphoramidothioate Modification referred as Hapten B; Fenthion
Derivative; Oxidized Fenthion; Fenthion Derivative; Oxidized Fenthion; pirimiphos-ethyl;
4-(Methylthio)-m-cresol; Chlorpyrifos Derivative; Chlorpyrifos-oxon; Fenchlorphos;
O,O-dimethyl O-2,4,5-trichlorophenyl phosphorothioate; Trichloronate; O-Ethyl O-2,4,5-
trichlorophenyl ethyl-phosphonothioate; Dichlofenthion; O-2,4-dichlorophenyl O,O-diethyl
phosphorothioate; Parathion; O,O-diethyl O-4-nitrophenyl phosphorothioate; Thiophos;
Chlorpyrifos Derivative Modification Synthesis of AR1 is described; Chlorpyrifos Derivative;
O-Ethyl O-(3,5,6-Trichloro-2-pyridyl) O-(3-Carboxypropyl)Phosphorothioate;
(PO); Chlorpyrifos Derivative - O-Ethyl O-(3,5,6-Trichloro-2-pyridyl) N-(5-
Carboxyethyl)Phosphoramidothioate; (PN1) (Modification Amide linkage of thiophosphate
reagents); Chlorpyrifos Derivative; O-Ethyl O-(3,5,6-Trichloro-2-pyridyl) N-(2-
Carboxyethyl)Phosphoramidothioate; (PN1) (Modification Amide linkage of suitable
thiophosphate reagents); Triadimefon; (RS)-1-(4-chlorophenoxy)-3,3-dimethyl-1-(1H-
1,2,4-triazol-1-yl)butan-2-one; GR151004; (4-[[5-[3-[2-(dimethylamino)ethyl]]-5-
benzofuranyl]-3-pyridinyl]acetyl]morpholine dihydrochloride; Diflubenzuron;
1-(4-chlorophenyl)-3-(2,6-difluorobenzoyl)urea; (Structurally related s-triazines) - SprAAT
(Modification R1 = SCH2CH2COOH R2 = NH2 R3 = NH2); (Structurally related
s-triazines); SBeAAT (Modification R1 = S(C6H4)COOH R2 = NH2 R3 = NH2);
(Structurally related s-triazines); SAAT (Modification R1 = SH R2 = NH2 R3 = NH2);
(Structurally related s-triazines); CDAT (Modification R1 = Cl R2 = NH[C(O)CH3)
R3 = NH2); (Structurally related s-triazines)- CDET (Modification R1 = Cl
R2 = NH[C(O)CH3) R3 = NH(CH2CH3); (Structurally related s-triazines) - CDIT
(Modification R1 = Cl R2 = NH[C(O)CH3) R3 = NH(CH(CH3)2)); (Structurally related
s-triazines); CDDT (Modification R1 = Cl R2 = NH[C(O)CH3) R3 = NH[C(O)CH3));
(Structurally related s-triazines) - ammeline; OAAT (Modification R1 = OH R2 = NH2
R3 = NH2); (Structurally related s-triazines)- ammelide; OOAT (Modification R1 = OH
R2 = OH R3 = NH2); (Structurally related s-triazines) - cyanuric acid; OOOT (Modification
R1 = OH R2 = OH R3 = OH); (Structurally related s-triazines); melamine; AAAT
(Modification R1 = NH2 R2 = NH2 R3 = NH2); Structurally related s-triazines-
N-isoropylammeline; OIAT (Modification R1 = OH R2 = NH[CH(CH3)2] R3 = NH2;
Structurally related s-triazines - N-ethylammeline; OEAT (Modification R1 = OH
R2 = NHCH2CH3 R3 = NH2); Structurally related s-triazines; N-ethylammelide; OOET
(Modification R1 = OH R2 = OH R3 = NHCH2CH3); Structurally related s-triazines)-
cyromazine,CyPAAT (Modification R1 = NH(C3H5) R2 = NH2 R3 = NH2); Structurally
related s-triazines - diamino-s-triazine;, HAAT (Modification R1 = H R2 = NH2 R3 = NH2);
PCB congeners; 2,5,3′,4′-tetrachlorobiphenyl (Modification IUPAC no.: 70); PCB congeners
2,4,5,3′,4′-pentachlorobiphenyl (Modification IUPAC no.: 118); PCB congeners - 2,2′,5,5′-
tetrachlorobiphenyl (Modification IUPAC no.: 52); PCB congeners; 6-[3,3′,4′-
Trichlorobiphenyl-4-yl)oxy]hexanoic Acid; Metolazone; Brand Names: Mykrox;
Zaroxolyn; Furfuryl benzoate; DDT Metabolites; DDA; Paraquat; 1,1′-dimethyl-4,4′-
bipyridinium ion; Diethylcarbamazine; THP; 2,4,6-triphenyl-N-(4-hydroxyphenyl)-
pyridinium; o-DNCP; -dinitrocarboxyphenol; PCB congeners; 3-chlorobiphenylol
(Modification IUPAC No. 2); PCB congeners; 3,4′-dichlorobiphenyl (Modification IUPAC
No. 13), PCB congeners; 3,5-dichlorobiphenyl (Modification IUPAC No. 14); PCB congeners;
3,4,5,3′,4′-pentachlorobiphenyl (Modification IUPAC No. 126); 2,3,3′,4′-tetrachlorobiphenyl
(Modification IUPAC No. 56); 2′,3,4,5-tetrachlorobiphenyl (Modification
IUPAC No. 76); 3,3′,5,5′-tetrachlorobiphenyl (Modification IUPAC No. 80);
2,4,5,2′,5′-pentachlorobiphenyl (Modification IUPAC No. 101); 2,3,3′,4,4′-
pentachlorobiphenyl (Modification IUPAC No. 105); 2,3,6,3′,4′-pentachlorobiphenyl
(Modification IUPAC No. 110); 3,3′,4,5,5′-pentachlorobiphenyl (Modification IUPAC No.
127); 3,4,5,3′,4′,5′-hexachlorobiphenyl (Modification IUPAC No. 169); 2,3,3′,4,4′,5-
hexachlorobiphenyl (Modification IUPAC No. 156); 3,4,3′,4′-tetrabromobiphenyl;
3,4,5,3′,4′,5′-hexabromobiphenyl; 2,4,5,2′,4′,5′-hexabromobiphenyl; Dibenzofurans and
Dioxins; 2,3,7,8-tetrachlorobenzofuran; 2,3,7,8-tetrachlorodibenzo-p-dioxin; 3,4′,5- trichloro-
4-biphenylol; 3,3′,5,5′-tetrachloro-4,4′-biphenyldiol; 3,4,3′,4′-tetrachlorodiphenyl ether;
1-2-dichlorobenzene; 1,4-dichlorobenzene; 1,2,4-trichlorobenzene;
3,4-dichloroaniline; DDT Metabolites; 4,4′-DDT; 4,4′-DDD Retronecine;
3,4-dichlorobiphenyl Modification IUPAC No. 12,; 3,4,3′-trichlorobiphenyl
(Modification IUPAC No. 35); PCB Congeners; 3,4,4′-trichlorobiphenyl (Modification
IUPAC No. 37); 3,4,3′,5-tetrachlorobiphenyl (Modification IUPAC No. 78); 3,4,3′,5′-
tetrachlorobiphenyl (Modification IUPAC No. 79); 3,4,4′,5-tetrachlorobiphenyl
(Modification IUPAC No. 81); DDT Metabolites; p,p′-DDT (Modification p,p′-
dichlorodiphenyltrichloroethane); o,p′-DDT Modification o,p′-
dichlorodiphenyltrichloroethane; p,p′-DDE Modification p,p′-DDE; o,p′-DDE
Modification o,p′-; p,p′-DDD Modification p,p′-DDD; o,p′-DDD Modification o,p′-DDD;
Dicofol; 4,4-dichloro-a-(trichloromethyl)benzhydrol; Cyprazine; 6-chloro-N-cyclopropyl-
N′-(1-methylethyl)-1,3,5-triazine-2,4-diamine; Structurally related s-triazines; Dipropetryn;
6-(ethylthio)-N,N′-bis(1-methylethyl)-1,3,5-triazine-2,4-diamine; Trietazine; 6-chloro-
N,N,N′-triethyl-1,3,5-triazine-2,4-diamine; 6-Hydroxyatrazine; hexazinone; 3-cyclohexyl-
6-dimethylamino-1-methyl-1,3,5-triazine-2,4(1H,3H)-dione; TNT; 2,4,6-Trinitrotoluene;
Tetraconazole (M14360); 1-[2-(2,4-dichlorophenyl)-3-(1,1,2,2-tetrafluoroethoxy)propyl]-
1H-1,2,4-triazole; DTP; 2-(2,4-dichlorophenyl)-3-(1H-1,2,4-triazol-1-yl)propanol; Imazalyl;
fenarimol; (RS)-2,4′-dichloro-α-(pyrimidin-5-yl)benzhydryl alcohol; Lupanine metabolites;
(+)-lupanine (Modification R = H); Lupanine metabolites; (+)-13-hydroxylupanine
(Modification R = OH); Lupanine metabolites; hemisuccinate ester of (+)-13-hydroxylupanine
(Modification R = OCO—(CH2)2•COOH); Lupanine metabolites; cis-hexahydrophthalate
ester of (+)-13-hydroxylupanine (Modification R = OCO•C6H10•COOH);
Lupanine metabolites; alpha-isolupanine; Lupanine metabolites; -hydroxylupanine;
Sparteine; Cysteine; multiflorine; epilupinine; (Structurally related s-triazines); CYANAZINE
ACID Modification R1 = Cl R2 = NHCH2CH3 R3 = NHCCOOH(CH3)2;
Structurally related s-triazines Modification R1 = Cl R2 = NHCH2CH3
R3 = NH(CH2)3COOH; Structurally related s-triazines (Modification R1 = Cl
R2 = NHCH2CH3 R3 = NHCH2COOH); (Structurally related s-triazines)
(Modification R1 = Cl R2 = NHCH2CH3 R3 = NH(CH2)4COOH); norflurazon;
4-chloro-5-(methylamino)-2-[3-(trifluoromethyl)phenyl]-3(2H)-pyridazinone;
norflurazon derivative; desmethyl-norflurazon; metflurazon; -chloro-5-(dimethylamino)-2-
[(3-trifluoromethyl)phenyl]-3(2H)- pyridazinone; Pyrazon; Chloridazon; 5-amino-4-chloro-2-
phenyl-3(2H)-pyridazinone (active ingradient); dichlorophenyl-pyridazone; (Structurally
related s-triazines) azidoatrazine (Modification R1 = N3 R2 = NHCH(CH3)2
R3 = NHCH2CH3); ALACHLOR 2-chloro-2′,6′-diethyl-N-methoxymethylacetanilide;
trichothecolone (Modification R1 = H R2 = OH R3 = H R4 = O R5 = H); DON derivative;
acetyl-T-2; DON derivative; T-2 tetrol tetraacetate; Chlorpyrifos derivatives;
mono-dechloro-CP; Bromophos derivative; Bromophos-methyl; Bromophos derivative; Bromophos-ethyl
dicapthon; -2- chloro-4-nitrophenyl O,O-dimethyl phosphorothioate; tetrachlorvinphos; (Z)-2-
chloro-1-(2,4,5-trichlorophenyl)vinyl dimethyl phosphate; triclopyr; 3,5,6-trichloro-2-
pyridyloxyacetic acid; picloram; 4-amino-3,5,6-trichloropyridine-2-carboxylic acid;
Formononetin; Biochanin A; 5; 7-dihydroxy-4′-methoxyisoflavone (Modification It is the
4′-methyl ether of genistein); equol; (7-hydroxy-3-(4′-hydroxyphenyl)-chroman;
2′methoxyformononetin; Daidzein; 7-hydroxy-3- (4-hydroxyphenyl)-4H -1-benzopyran-4-
one; geninstein; quercetin; 3,3′,4′,5,7-Pentahydroxyflavone; 3,5,7,3′,4′-
Pentahydroxyflavone;; matheucinol; coumestrol; (Structurally related s-triazines);
Hydroxysimazine (Modification R1 = OHR2 = NHCH2CH3R3 = NHCH2CH3;
angustifoline; Alodan; 1 - Methyl - 4 - phenyl - 4 - carboethoxypiperidine hydrochloride;
Zearalenone; RAL; F-2 Toxin; Fenpropimorph; (RS)-cis-4-[3-(4-tert-butylphenyl)-2-
methylpropyl]-2,6-dimethylmorpholine; Tridemorph; 2,6-dimethyl-4-tridecylmorpholine;
2,6-dimethylmorpholine; Amorolfine; Fenpropidin; (RS)-1-[3-(4-tert-butylphenyl)-2-
methylpropyl]piperidine; (Structurally related s-triazines) (Modification R1 = Cl R2 = Cl
R3 = NHCH2CH3; (Structurally related s-triazines) Modification R1 = Cl R2 = Cl
R3 = NHCH(CH3)2; (Structurally related s-triazines) Modification R1 = Cl
R2 = NHCH2CH3 R3 = NH(CH2)5COOH; (Structurally related s-triazines) Modification
R1 = Cl R2 = NHCH(CH3)2 R3 = NHCH2COOH; (Structurally related s-triazines)
(Modification R1 = Cl R2 = NHCH(CH3)2 R3 = NH(CH2)5COOH); Structurally
related s-triazines; cyanazine amide (Modification R1 = Cl R2 = NHCH2CH3
R3 = NHCCONH2(CH3)2); hydroxycyanazine acid (Modification R1 = OH
R2 = NHCH2CH3 R3 = NHCCOOH(CH3)2); deethylsimazine (Modification R1 = Cl
R2 = NH2 R3 = NHCH2CH3); Albendazole sulfoxide; [5-(propylthionyl)-1H-benzimidazol-2-yl]-,
methylester; Albendazole sulfone; 5(6)-alkylbenzimidazoles; 2-amino-5-
(propylthio)benzimidazole; 5(6)-alkylbenzimidazoles; 2-amino-5-
(propylsulfonyl)benzimidazole; oxibendazole; 5-propoxy-benzimidazole-2-methyl
carbamate; 5(6)-arylbenzimidazoles; fenbendazole sulfone (Modification sulfone
metabolite of fenbendazole); 5(6)-arylbenzimidazoles; 4′-hydroxyfenbendazole;
5(6)-arylbenzimidazoles; oxfendazole (Modification Oxfendazole is the sulfoxide
metabolite of fenbendazole); 5(6)-arylbenzimidazoles; flubendazole; benzimidazole
Metabolites; 2-aminobenzimidazole; benzimidazole Metabolites; 5-aminobenzimidazole;
benzimidazole Metabolites; 2-acetylbenzimidazole; Benzophenone; Diphenylmethanone;
phenyl ketone; Diphenyl ketone; Benzoylbenzene; Benzaldehyde; benzoic aldehyde; 4-Bromo-2,5-
dichlorophenol; Acephate; O,S-dimethyl acetylphosphoramidothioate; methamidophos;
O,S-dimethyl phosphoramidothioate; Dichlorvos; 2,2-dichlorovinyl dimethyl phosphate;
Phenthoate; S-α-ethoxycarbonylbenzyl O,O-dimethyl phosphorodithioate; EPN; Ethyl
p-nitrophenyl thionobenzenephosphonate; Bioresmethrin; -benzyl-3-furylmethyl (1R,3R)-
2,2-dimethyl-3-(2-methylprop-1-enyl)cyclopropanecarboxylate (Modification The
unresolved isomeric mixture of this substance has the ISO common name resmethrin);
flufenoxuron; 1-[4-(2-chloro-α,α,α-trifluoro-p-tolyloxy)-2-fluorophenyl]-3-(2,6-
difluorobenzoyl)urea; Amitrole; 1H-1,2,4-triazol-3-ylamine; molinate; S-ethyl azepane-1-
carbothioate; molinate derivative (Modification S-2-carboxyethyl hexahydroazepine-1-
carbothioate); molinate derivative (Modification S-5-carboxypentyl hexahydroazepine-1-
carbothioate) molinate derivative (Modification molinate sulfone); molinate derivative
(Modification S-(p-aminobenzyl) hexahydroazepine-1-carbothioate); molinate derivative
(Modification S-2-(p-aminophenyl)ethyl hexahydroazepine-1-carbothioate);
hexamethylenimine; thiobencarb (Bolero); butylate (Sutan); EPTC (Eptam); cycloate
(Roneet); pebulate (Tillam); vernolate (Vernam); Aflatoxin M1; AFM1 (Modification
AFM1); Aflatoxin B1; AFB1 (Modification AFB1); Aflatoxin G1; AFG1 (Modification
AFG1); Aflatoxin M2; AFM2 (Modification AFM2); Aflatoxin B2; AFB2 (Modification
AFB2); Aflatoxin G2; AFG2 (Modification AFG2); Aflatoxin B2alpha; AFB2alpha
(Modification AFB2alpha); Aflatoxin G2alpha; AFG2alpha (Modification AFG2alpha);
KB-6806; 6-amino-5-chloro-1-isopropyl-2-(4-methyl-1-piperazinyl) (Modification
R1 = NH2 R2 = CH(CH3)2 R3 = CH3); KB-6806 (Benzimidazole) Derivatives
Modification R1 = NH2 R2 = CH2CH(CH3)2 R3 = CH3; Hapten Name KB-6806 (Benzimidazole)
Derivatives (Modification R1 = NH2 R2 = CH(CH2CH3)2 R3 = CH3); KB-6806
(Benzimidazole) Derivatives (Modification R1 = NHCOCH3 R2 = CH(CH3)2 R3 = CH3);
KB-6806 (Benzimidazole) Derivatives (Modification R1 = H R2 = CH(CH3)2 R3 = CH3);
KB-6806 (Benzimidazole) Derivatives (Modification R1 = NH2 R2 = CH(CH3)2 R3 =
CH3); KB-6806 (Benzimidazole) Derivatives Modification R1 = NH2 R2 = CH(CH3)2
R3 = = N(−>0) CH3 (N-OXIDE); KB-6806 (Benzimidazole) Derivatives Modification
R1 = NH2 R2 = CH(CH3)2 R3 = H; KB-6806 (Benzimidazole) Derivatives Modification
R1 = NH2 R2 = CH2CH3 R3 = CH3; Aminopraoxon; phosphoric acid; O,O-diethyl O-(4-
aminophenyl) ester,Methylparathion; phosphorothioic acid; O,O-dimethyl O-(4- nitrophenyl)
ester; Diethyl phenylphosphate; phenylphosphonic acid; O,O-diethyl ester;
Diethyl phosphate; ethylphosphonic acid; O,O-diethyl ester; p-Nitorphenyl phosphate;
phosphonic acid; O-(4-nitrophenyl)ester; Phorate; phosphorodithioic acid; O,O-diethyl
S-[(ethylthio)methyl] ester; Ethion; bis(phosphorodithioic acid); S,S′-methylene O,O,O′,O′-
tetraethyl ester; Carbophenthion; phosphorodithioic acid; O,O-diethyl S-[[(4-
chlorophenyl)thio]methyl] ester; Disulfoton; phosphorodithioic acid; O,O-diethyl S-[(2-
ethylthio)ethyl] ester; TS; N-[4-(Carboxymethyl)-2-thiazolyl)sulfanilamide; NS; N-(4-
Nitrophenyl)sulfanilamide; Sulfamoxole; Sulfacetamide; DNP-SL; Spin labelled
dinitrophenyl (Modification The synthesis of DNP-SL has been described by Balakrishnan
et al (1982) formula can be found in Anglister et al. (1984)); beta ecdysone; Benzimidazole
Derivative; 5(6)-[Carboxypentyl)thio]-2-(methoxycarbonyl)amino]-benzimidazole;
2-hydroxybiphenyl; HBP; Atrazine Caproic acid; Lysophosphatidic acid (LPA); 1-acyl-2-
hydroxy-sn-glycero-3-phosphate); berberine; Palmatine; 9-Acetylberberine; Corydaline;
Coptisine; Berberrubine; 8-Oxoberbeine; Papaverine; Berberine Derivative; 9-O-carboxymethyl
berberine; phencyclidine; 1-(1-phenylcyclohexyl)piperidine; Methoxychlor;
Endosulfan Derivative; 4-Oxobutanoic Acid,4-(4,5,6,7,8,8-Hexachloro-3a,4,7,7a- tetrahydro-
4,7-methano-1H-indenyl-1-oxy); Endosulfan Derivative; 4-oxybutanoic Acid,4-
(1,3,4,5,6,7,8-Octachloro-3a,4,7,7a-tetrahydro-4,7-methanoindanyl-2-oxy; Endosulfan
Derivative (Modification Hemisuccinate of Endosulfan diol); Triazole Derivatives; 5-(3-
Hydroxypropyl)-3-amino-2H-1,2,4-triazole; Triazole Derivatives; 5-(3-Hydroxypropyl)-3-
(2-nitrophenylsulfenyl)amino-2H-1,2,4-triazole; Triazole Derivatives; 3-Amino-5-[(3-
succinyloxy)propyl]-2H-1,2,4-triazole; Triazole Derivatives; 3-amino-1,2,4-triazole-5-thiol;
Triazole Derivatives; 3-[(2-nitrophenylsulfenyl)amino-2H-1,2,4-triazole-5-thiol;
Triazole Derivatives; 2H-1,2,4-triazole-5-thiol; Triazole Derivatives; 4-methyl-1,2,4-
triazole-3-thiol; Triazole Derivatives; (1,2,4-triazol-2-yl)acetic acid; 1,2,4-triazole; 4-
nitrophenyl 4′-carboxymethylphenyl phosphate; Triazole Derivative; 4-amino-1,2,4-
triazole; Triazole Derivative; 3-acetamido-1H-1,2,4-triazole; Triazole Derivative; 3-amino-
1,2,4-triazole-5-carboxylic acid hemihydrate; Triazole Derivative; 2-(4-chlorophenyl)-2-
(1,2,4-triazol-1-yl)-methylhexanoic acid; succinic acid; Imidazole; L-histidine; L-glutamic
acid; Permethrin derivative; 3-phenoxybenzyl 2,2-dimethylcyclopropatane-1,3-dicarboxylate;
3-phenoxybenzaldehyde; flucythrinate; Chrysanthemic acid; 2,4-Dinitrophenyl; DNP;
Thiram Haptens; Disodium 4-[Carbodithioato(methyl)- amino]butanoate;
Thiram Haptens 5,11-Dimethyl-6,10-dithioxo-7,9-dithia-5,11-diazadodecanoic Acid;
Thiram Haptens; 2-{[(Dimethylamino)carbothioyl]sulfanyl}ethanoic Acid;
Thiram Haptens; 4-{[(Dimethylamino)carbothioyl]sulfanyl}butanoic Acid;
Thiram Haptens; 6-{[(Dimethylamino)carbothioyl]sulfanyl}hexanoic Acid;
Thiram Haptens; 11-{[(Dimethylamino)carbothioyl]sulfanyl}undecanoic Acid;
Thiram Haptens; 2-{[(Dimethylamino)carbothioyl]sulfanyl}ethanoic Acid; Thiram;
Tetramethylthiurammonosulfide; Tetraethylthiuram disulfide; Dimethyldithiocarbamic acid
sodium salt; Dimethyldithiocarbamic acid zinc salt; Diethyldithiocarbamic acid sodium salt;
N,N,N′,N′-tetramethylthiourea; Nabam; Zineb; Maneb; Ethylenethiourea; Chlorpyrifos
hapten; O,O Diethyl O-[3,5-Dichloro-6-[(2-carboxyethyl)thio]-2-pyridyl]
Phosphorothioate; 2-Succinamidobenzimidazole; Methyl 2-Benzimidazolecarbamate;
MBC; Benzimidazole; 2-benzimidazolylurea; succinamide; Ethyl carbamate; Urea; N-methylurea;
N,N′-dimethylurea; Brevetoxin PbTx-3; Organophosphorous Haptens; O,O-Diethyl
O-(5-carboxy-2-fluorophenyl) phosphorothioate; Chlorpyrifos-ethyl; Anandamide hapten;
N-Arachidonyl-7-amino-6-hydroxy-heptanoic acid; Anandamide; Arachidonic acid;
Docosatetraenoyl ethanolamide; Dihomo-gamma-linolenyl ethanolamide; 2- Arachidonyl
glycerol; 2-Arachidonyl glycerol ether; Stearoyl ethanolamide; Heptadecanoyl
ethanolamide; Prostaglandin E1; 3-hydroxy-2-(3-hydroxy-1-octenyl)-5- oxocyclopentaneheptanoic
acid; alprostadil; PGE1; Prostaglandin D2; PGD2; Prostaglandin A2; PGA2;
Prostaglandin B2; PGB2; Prostaglandin F2 alpha; 7-[3,5-dihydroxy-2-(3-
hydroxy-1-octenyl)cyclopentyl]-5-heptenoic acid; dinoprost; PGF2alpha; Prostaglandin F1
alpha; PGF1alpha; 6-keto-Prostaglandin F1 alpha; 6-keto-PGF1alpha; 13,14-Dihydro-15-
keto-Prostaglandin E2; 13,14-Dihydro-15-keto-PGE2; 13,14-Dihydro-15-keto-Prostaglandin
F2alpha; 14-Dihydro-15-keto-PGF2alpha; 5alpha,7alpha-Dihydroxy-11-ketotetranorpostane-
1,16-dioic acid; 15-keto-PGF2alpha; TXB2; Prostaglandin E2; 7-[3- hydroxy-2-(3-hydroxy-
1-octenyl)-5-oxocyclopentyl]-5-heptenoic acid; dinoprostone; PGE2;
hCG-alpha-(59-92)-peptide (34 residues); Paraquat Derivative; Paraquat hexanoate
(PQ-h); Monoquat; Diquat; 9,10-dihydro-8a,10a-diazoniaphenanthrene; MPTP; 1-methyl-4-
phenyl-1,2,5,6-tetrahydropyridine; 1,2-Naphthoquinone; N-Acetyl-S-(1,2-dihydroxy-4-
naphthyl)cysteine; N-Acetyl-S-(1,4-dihydroxy-2-naphthyl)cysteine; N-Acetyl-S-(1,2-
dihydroxy-1-hydroxy-1-naphthyl)cysteine; 2-Chloro-2′.6′-diethylacetanilide(CDA)
Hapten; 2-[2-Chloro-(2′-6′-diethyl)acetanilido]ethanoic Acid; 2-Chloro-2′.6′-
diethylacetanilide(CDA) Hapten; 2-[2-Chloro-(2′-6′-diethyl)acetanilido]butanoic Acid;
2-Chloro-2′ .6′-diethylacetanilide(CDA) Hapten; 5-(4-Chloroacetamido-3,5-
diethyl)phenoxypentanoic Acid; CDA; 2-Chloro-2′.6′-diethylacetanilide; HDA;
2- Hydroxy-2′.6′-diethylacetanilide; 2,6-diethyl-aniline; Hydroxyalachlor; Alachlor ESA;
Alachlor ethanesulfonic acid; Isoproturon Hapten; 3-(4-Isopropylphenyl)-1-carboxypropyl-
1-methyl urea; chlorotoluron; 3-(3-chloro-p-tolyl)-1,1-dimethylurea; Metoxuron; 3-(3-
chloro-4-methoxyphenyl)-1,1-dimethylurea; metamitron; 4-amino-4,5-dihydro-3-methyl-6-
phenyl-1,2,4-triazin-5-one; mecoprop; (RS)-2-(4-chloro-o-tolyloxy)propionic acid;
propyzamide; 3,5-dichloro-N-(1,1-dimethylpropynyl)benzamide; Paraquat dichloride; MCPB;
4-(4-chloro-o-tolyloxy)butyric acid; Chlortoluron Hapten; N-(3-Chloro-4-
methylphenyl)-N-methyl-N-carboxypropyl Urea; Metsulfuron; Methyl 2-[3-(4-methoxy-6-
methyl-1,3,5-triazin-2-yl)ureidosulphonyl]benzoate; Captopril Haptens; Captopril-4-
(Malelmidomethyl)-cyclohexane Carboxylic Acid(MCC); Captopril Haptens; Captopril
Disulfide Modification; Mercaptoethanol-MCC; Mercaptoethanol-4-(Malelmidomethyl)-
cyclohexane Carboxylic Acid Modification, Captopril Haptens; Captopril without MCC;
Aculeatiside A; Aculeatiside B; Solamargine; Solasonine; solanine-S; purapurine;
Solasodine; Khasianine; Tomatine; lycopersicin; Tomatidine; 3-O--beta-D-
Glucopyranosyl-solasodine; O-alpha-L-Rhamnosyl-1(1 −> 2)-3-O-beta-D-glucopyranosyl-
solasodine; 3-O-beta-D-Galacopyranosyl-solasidine; O-beta-D-Glucopyranosyl-1(1 −> 3)-3-
O-beta-D-galacopyranosyl-solasodine; 12-Hydroxysolamargine; 12-Hydroxysolasonine;
Isoanguivine; Solaverine I; Solaverine II; Xylosyl-beta-solamargine; alpha-Solanine;
alpha-Chaconine; Dioscine; Indole Derivatives; beta-indole Acetic Acid; 2-Bromo-4,6-
dinitroaniline; 2-Chloro-4,6-dinitroaniline; Tetryl; 2,4,6-trinitrophenyl-n-methylnitramine;
nitramine; tetralite; tetril; 2-Amino-4,6-dinitrotoluene; 2,4-Dinitroaniline; 3,5-Dinitroaniline;
2-Amino-4,6-dinitrobenzoic acid; Disperse Blue 79; N-[5-[bis[2-(acetyloxy)ethyl]amino]-
2-[(2-bromo-4,6-dinitrophenyl)azo]-4-ethoxyphenyl]acetamide; 1,3-Dinitrobenzene;
2,6-Dinitrotoluene; 4-Amino-2,6-dinitrotoluene; 1,3,5-Trinitrobenzene; Nicergoline;
Ethylmorphine; ,8-Didehydro-4,5-epoxy-3-ethoxy-17- methylmorphinan-6-ol;
Dihydromorphine; Dihydrocodeine; dihydromorphinone; Hydromorphone;
Dihydrocodeinone; Hydrocodone; Naltrexone; N-cyclopropylmethyl-14-
hydroxydihydromorphinone; Dextromethorphan; (±)-3-Methoxy-17-methylmorphinan;
Homatropine; Endorphins Modification Derivative Type: b-Endorphin; Met-enkephalin;
DALEA; D-Ala(2)-D-Leu(5)-enkephalinamide; Vincristine; 22-Oxovincaleukoblastine;
leurocristine; VCR; LCR; OCT; 22-Oxacalcitriol; OCT-3-HG; 22-oxacalcitriol-3-Hemiglutarate;
24(OH)OCT; 24(OH)-22-oxacalcitriol; 1,20(OH)2-hexanor-D3; Synephrine; Epinephrine;
4-[(1R)-1-Hydroxy-2-(methylamino)ethyl]-1,2-benzenediol; Phenylephrine;
Dopamine Derivative; 6-hydroxy dopamine; Tyramine derivative; 3-methoxy tyramine;
Phenethylamine; Benzeneethanamine; PEA; m-tyramine; o-tyramine;
dimethoxyphenethylamine; Thymidine glycol monophosphate; 5,6-Dihydroxythymidine
monophosphate; Thymidine monophosphate; Thymidine glycol; Thymine glycol; 5,6-
Dihydrothymidine; Thymidine; Thymine; 5-methyluracil; 2,4-dihydroxy-5-methylpyrimidine;
AMP; Adenosine mono phosphate; CMP; Cytidine mono phosphate; Carbamazepine;
5-carbamoyl-5H-dibenz[b,f]azepine; Neopterin isomers; D-erythro-Neopterin;
Neopterin isomers; L-erythro-Neopterin; Neopterin isomers; D-threo-Neopterin;
Biopterin isomers; L-erythro-Biopterin; Biopterin isomers; D-erythro-Biopterin; Biopterin
isomers; L-threo-Biopterin; Biopterin isomers; D-threo-Biopterin; Pterin-6-Carboxylic
Acid; C7H5NiO3; Pterin; Thromboxane B2; (5Z,9alpha,13E,15S)-9,11,15- trihydroxythromboxa-
5,13-dien-1-oic acid; 15 Ketoprostaglandin F2alpha; Fumonisin B1; macrofusine; FB1;
Thyroliberin; TRH; thyrotropin-releasing factor; thyrotropin releasing hormone;
TRF; protirelin; lopremone; Thyroliberin-OH; TRH-OH; Diketopiperazine; cyclo
(H-P); TRH analogues; Methylated TRH; TRH analogues; TRH elongated peptides; TRH-
Gly; TRH elongated peptides; TRH-Gly-Lys-Arg; TRH elongated peptides; TRH-Gly-Lys-
Arg-Ala; TRH elongated peptides; P7 (Modification Q-H-P-G-L-R-F); TRH elongated
peptides; P10 (Modification S-L-R-Q-H-P-G-L-R-F); TRH elongated peptides; Ps5
Modification pro-TRH[178-199]; TRH elongated peptides; TRH-Ps5 (Modification pro-
TRH[172-199]; Hypothalmic peptide; LHRH; Cyanoginosin-LA; Cyanoginosin-LB;
Cyanoginosin-LR; Cyanoginosin-LY; Cyanoginosin-AY; Cyanoginosin-FR;
Cyanoginosin-YR; Ne-acetyllysine-containing peptide; Gly-Lys(Ac)-e-aminocaproic acid
(Aca)-Cys; Benzoic Acid; Benzenecarboxylic acid; phenylformic acid; dracylic acid;
m-hydroxybenzoic acid; 3-hydroxybenzoic acid; o-methoxybenzoic acid; 2-methoxybenzoic
acid; o-toluic acid; 2-Methylbenzoic acid; o-chlorobenzoic acid; 2-chlorobenzoic acid;
o-aminobenzoic acid; 2-aminobenzoic acid; thiosalicylic acid; 2-Mercaptobenzoic acid;
o-sulfhydrylbenzoic acid; Salicylamide; 2-Hydroxybenzamide; Saligenin; saligenol;
o-hydroxybenzyl alcohol; Salicyl alcohol; 2-cyanophenol; 2-hydroxyphenyl acetic acid;
p-hydroxybenzoic acid; p-aminobenzoic acid; 4-Aminobenzoic acid; vitamin Bx; bacterial
vitamin H1; p-toluic acid; p-methylamino benzoic acid; p-chlorosalicylic acid;
4-chloro-2-hydroxybenzoic acid; 2,4-dihydroxybenzoic acid; beta-Resorcylic Acid;
2,4-dihydroxybenzenecarboxylic acid; BRA; 4-aminosalicylic acid; 4-Amino-2-hydroxybenzoic
acid; p-aminosalicylic acid; Gentisic Acid; 2,5-dihydroxybenzoic acid; 5-hydroxysalicylic
acid; Picolinic acid; o-Pyridinecarboxylic acid; 2-Pyridinecarboxylic acid; picolinic acid
N-oxide; 3-hydroxypicolinic acid; 2-hydroxynicotinic acid; 7-methylguanine;
N2-Carboxymethyl-N7-methylguanine; 2-(7-methyl-6-oxo-6,7-dihydro-1H-purin-2-
ylamino)acetic acid; 7-methylxanthine; 7-methyluric acid; 7-methyladenine; Guanine;
2-Amino-1,7-dihydro-6H-purin-6-one; 2-aminohypoxanthine; Adenine; 6-aminopurine;
6-amino-1H-purine; 6-amino-3H-purine; 6-amino-9H-purine; 7-(2-Carboxyethyl)guanine;
7-CEGua; 7-Ethylguanine; 2-amino-7-ethyl-1H-purin-6(7H)-one; 7-(2,3-Dihydroxypropyl)guanine;
2-amino-7-(2,3-dihydroxypropyl)-1H-purin-6(7H)-one; 7-(2-Hydroxyethyl)guanine;
2-amino-7-(2-hydroxyethyl)-1H-purin-6(7H)-one; 7-(2-[(2- Hydroxyethyl)amino]ethyl)-
guanine; 2-amino-7-(2-(2-hydroxyethylamino)ethyl)-1H-purin-6(7H)-one;
7-Carboxymethylguanine; 2-(2-amino-6-oxo-1,6-dihydropurin-7-yl)acetic acid;
fluorescein; urushiol; quinone; biotin; His-tags; FLAG-tags; Strep-tag; Myc-tag;
HA-tag; Spot-tag; or NE-tag or any combination thereof.

In some embodiments, the hapten comprises fluorescein or a derivative thereof.

In some embodiments, the hapten comprises DNP or a derivative thereof.

“Target moiety” has its plain and ordinary meaning when read in light of the specification, and may include but is not limited to, for example, a specific group or site on a molecule or chemical that is a binding target for another chemical or protein of interest. In some alternatives described herein, the target moiety is a hapten. Examples of haptens useful with embodiments provided herein are listed in TABLE 1. In some alternatives, the CAR comprises an antibody or a portion thereof, such as one or more binding domains or comprising one or more CDRs. Non-limiting examples of antibodies or antigen binding portions thereof useful with embodiments provided herein include antibodies against the haptens listed in TABLE 1, and the antibodies listed in TABLE 2.

TABLE 2
EXAMPLE ANTIBODIES USEFUL FOR EMBODIMENTS HEREIN

Anti 3-methylindole antibody; 3F12; Anti 3-methylindole antibody; 4A1G; Anti 3-methylindole
antibody; 8F2; Anti 3-methylindole antibody; 8H1; Anti 3-methylindole antibody;
Anit-Fumonisin B1 antibody; Anti1,2-Naphthoquinone-antibody; Anti 15-Acetyldeoxynivalenol
antibody; Anti(2-(2,4-dichlorophenyl)-3(1H-1,2,4-triazol-1-yl)propanol) antibody (AntiDTP
antibody); Anti22-oxacalcitriol antibody (As-1; 2 and 3); Anti(24,25(OH)2D3) antibody
(Ab11); Anti(24,25(OH)2D3) antibody (Ab3); Anti(24,25(OH)2D3) antibody (Ab3-4);
Anti2,4,5-Trichlorophenoxyacetic acid antibody; Anti (2,4,5-Trichlorphenoxyacetic acid)
antibody; Anti(2,4,6-Trichlorophenol) antibody; Anti(2,4,6-Trichlorophenol) antibody;
Anti 2,4,6-Trinitrotoluene(TNT) antibody; Anti2,4-Dichlorophenoxyacetic acid(MAb′s
B5/C3; E2/B5; E2/G2; F6/C10; and F6/E5); Anti (2,4- Dichlorphenoxyacetic acid)
antibody; Anti2-hydroxybiphenyl-antibody; Anti(3,5,6- trichloro-2-pyridinol) antibody
(LIB-MC2; LIB-MC3); Anti (3,5,6-trichloro-2-pyridinol) antibody (LIB-MC2 MAb);
Anti3-Acetyldeoxynivalenol(3-AcDON) antibody; Anti3- phenoxybenzoic acid
(3-PBAc)-antibody; Anti -4-Nitrophenol antibody; anti4-nitrophenyl
4′-carboxymethylphenyl phosphate antibody; Anti7-(Carboxyethyl)guanine(7-CEGua)
antibody (group specific for 7-meGua); Anti7-methylguanine(7-MEGua) antibody;
AntiABA antibody; Anti Acephate antibody (Antiserum 8377); Antiacetyllysine antibody
(mAbs AL3D5; AL11; AKL3H6; AKL5C1); Anti Aculaetiside-A antibody; Anti Aflatoxin
M1(AFM1)antibody (mAbs A1; N12; R16; FF32); Antiagatharesinol antibody;
Antiagatharesinol antibody; Anti Amidochlorantibody; AntiAmitrole antibody (anti
1a-BSA antibody); Anti ampicillin antibody(AMPI I 1D1 and AMPI II 3B5); Antianandamide
antibody (9C11.C9C; 30G8.E6C; 7D2.E2b; 13C2 MAbs); Anti atrazine antibody;
Antiatrazine antibody; AntiAtrazine antibody; AntiAtrazine antibody; AntiAtrazine
antibody; AntiAtrazine antibody; AntiAtrazine antibody (4063-21-1 MAb cell line mAb and
scAbs); AntiAtrazine antibody (4D8 and 6C8 scAb); Anti Atrazine antibody (C193); Anti
Atrazine antibody (In Rabbit/Sheep); Anti Atrazine antibody (K4E7); Anti Atrazine
antibody (MAb: AM7B2.1); Anti Atrazine antibody(ScAb); Anti Atrazine Mercapturic
acid antibody; Anti (Azinphos methyl) antibody (MAB′s LIB-MFH14; LIB-MFH110);
Anti benalaxyl antibody; Anti bensimidazolecarboxylic acid; Anti benzimidazoles antibody
(Ab 587); AntiBenzo[a]pyrene antibody; Anti Benzo(a)pyrene antibody (10C10 and 4D5
MAbs); Anti(Benzoylphenylurea)-antibody (mainly against Diflubenzuron); Antiberberine
antibody; Antibeta Indole Acetic Acid antibody; AntiBiopterin(L-erythro form) antibody;
AntiBrevetoxin PbTx-3-antibody; Anti Bromacil antibody; AntiBromophos antibody;
AntiBromophos ethyl antibody; Anti Butachlor antibody; AntiCaptopril-MCC antibody;
AntiCarbamazepine(CBZ)- antibody; Anti Carbaryl antibody; Anti Carbaryl antibody
(LIB-CNH32; LIB-CNH33,LIB-CNH36; LIB-CNH37; LIB-CNH45; LIB-CNA38);
AntiCarbaryl antibody (LIB/CNH-3.6 MAb); Anti Carbofuran antibody(LIB-BFNB-52;
LIB-BFNB-62; LIB-BFNB-67); Anti Carbofuran antibody(LIB-BFNP21); AntiCDA-
antibody; AntiCDA-antibody (anti2-[2-Chloro-(2′-6′-diethyl)acetanilido]butanoic Acid);
AntiCDA-antibody (anti2-[2-Chloro-(2′-6′-diethyl)acetanilido]ethanoic Acid); AntiCDA-
antibody (anti 5-(4-Chloroacetamido-3,5-diethyl)phenoxypentanoic Acid); anticeftazidime
antibody; Anti(chlorodiamino-s-triazine)antibody (AntiCAAT) (PAb1-8); Anti Chlorothalonil
antibody; AntiChlorpyrifos antibody; AntiChlorpyrifos antibody; AntiChlorpyrifos
antibody(LIB-AR1.1; LIB-AR1.4 Mabs); AntiChlorpyrifos antibody
(LIB-C4); Anti (chlorpyrifos) antibody (LIB-C4 MAb); AntiChlorpyrifos antibody
(LIB-PN1 Mabs); AntiChlorpyrifos antibody(LIB-PN2 Mabs); AntiChlorpyrifos antibody
(LIB-PO Mabs); Antichlorsulfuron antibody; AntiChlorsulfuron antibody; Anti Chlortoluron
antibody (Antiserum); AntiCyanoginosin-LA antibody (mAbs 2B2-2; 2B2-7; 2B2-8; 2B2-9;
2B2-10; 2B5-5; 2B5-8; 2B5-14; 2B5-15; 2B5-23); Anti(D-3-methoxy-4-hydroxyphenylglycol)
antibody; AntiDDA antibody; Anti DDT antibody (PAbs and MAbs); AntiDDT Mabs
(LIB1-11; LIB5-21; LIB5-25; LIB5-28; LIB5-212; LIB5-51; LIB5-52; LIB5-53);
AntiDEC antibody (Anti diethylcarbamazine antibody); Anti DEHA antibody;
Anti(Delor 103) antibody; AntiDeltamethrin antibody; Anti Deltamethrin antibody (Del 01
to Del 12 MAbs and PAbs); Antideoxynivalenol(DON) antibody; AntiDeoxynivalenol(DON)
antibody; Anti Dexamethasone antibody; Anti Dexamethasone antibody;
AntiDinitrophenyl(DNP)-antibody; Anti dinitrophenyl spin labeled antibody (AN01-AN12);
Anti Diuron Antoboides (MAb′s: 21; 60; 195; 202; 275; 481; 488; 520);
Anti -D-MHPG antibody; Anti DNC antibody; AntiEB1089 antibody; Antiecdysone
antibody; Antiendosulfan antibody; AntiEndosulfan antibody; Anti Esfenvalerate antibody
(Ab7588); Anti estradiol antibody; AntiFenitrothion antibody (pAbs and mAbs);
AntiFenpropimorph antibody; Anti Fenthion antibody; AntiFenthion antibody; Anti FITC
antobodies (B13-DEI); AntiFlucofuron antibody(F2A8/1/A4B3); Antiflufenoxuron
antibody; and Anti(Benzoylphenylurea)-antibody; AntiFormononetin antibody;
AntiFurosemide antibody (Furo-26; Furo37; furo-72; Furo 73 Mabs); AntiGR151004
antibody; AntihCG-alpha-peptide antibody (FA36; Anti hydroxyatrazine antibody
(HYB-283-2); AntiHydroxysimazine antibody; Anti Imazalil antibody MoAb′s(9C1-1-1;
9C5-1-1; 9C6-1-1; 9C8-1-1; 9C9-1-1; 9C12-1-1; 9C14-1-1; 9C16-1-1; 9C18-1-1;
9C19-1-1; 9E1- 1; 9G2-1); Anti Irgarol antibody; Anti Isopentenyl adenosine antibody;
Anti Isoproturon antibody; AntiKB-6806 antiserum; Anti −(+)lupanine antibody; Anti
Lysiohosphatidic(LPA) acid; Anti M3G Ab1 and Ab2; Anti M3G Ab1 and Ab2; AntiMBC
antibody (Anti2-succinamidobenzimidazole antiserum); Anti Metanepharine antibody; anti
(+)methamphetamine antibody; Anti Methiocarb antibody (LIB-MXNB31; LIB-MXNB-33;
LIB-MXNH14 and LIB-MXNH-15 MAbs); Anti Metolachlor antibody; AntiMetolachlor
antibody; AntiMetolachlor antibody (MAb 4082-25-4); Anti Molinate antibody;
Anti monuron antibody; Antimorphine-3-glucuronide(E3 scFv antibody); Anti morphine
antibody; AntiMorphine antibody; AntiMorphine antibody (mAbs 8.2.1; 33.2.9;
35.4.12; 39.3.9; 44.4.1; 76.7F.16; 83.3.10; 115.1.3; 124.2.2; 131.5.13; 158.1.3; 180.2.4);
AntiNeopterin(D-erythro form) antibody; AntiNicarbazin antibody (Nic 6; Nic 7; Nic 8; and
Nic 9); Anti Nicergoline antibody(Nic-1; Nic-2; Nic-3 & BNA-1; BNA-3); Antinorflurazon
antibody; Anti NorMetanepharine antibody; Anti (o-DNCP) antibody; Anti - P10 antibody
(TRH elongated peptide); Anti Paraoxon antibody (BD1 and CE3); Anti Paraquat antibody;
AntiParaquat antibody; anti Parathion-methyl antibody; Anti PCB antibody (against
3,3′,4,4′-tetrachlorobiphenyl) MAb S2B1; Anti pentachlorophenol antibody; Anti
Pentachlorophenol antibody; AntiPentachlorophenol antibody; Anti permethrin antibody
(Mabs Py-1; Py-3 and Py-4); Anti Phencyclidine antibody (Mab 6B5 Fab); Antiphenobarbital
antibody; Antiphenobarbital antibody; Anti(p.p′-DDT)- antibody (LIB-DDT-35
and LIB-DDT5-52); Anti premethrin antibody(Ab549); Anti Propoxur antibody
(LIB-PRNP15; LIB-PRNP21; LIB-PRNB21; LIB-PRNB33); AmiProstaglandin E2-antibody;
Antip-tyramine antibody; Anti pyrene antibody; Anti retronecine antibody;
AntiRetronecine antibody; Anti salicylate antibody; Anti Sennoside A antibody(MAb 6G8);
Anti Sennoside B antibody(MAb′s: 7H12; 5G6; 5C7); Anti Simizine antibody; Anti
Sulfonamides antibody (AntiTS); AntiSulocfuron antibody(S2B5/1/C3); Anti
sulphamethazine antibody (21C7); Antisynephrine antibody; AntiThiabendazole antibody
(antibody 300); AntiThiabendazole antibody (antibody 430 and 448); AntiThiram-antibody;
Anti THP antibody (7S and 19S); Anti Thromboxane B2 antibody; Antithymidine glycol
monophosphate antibody (mAb 2.6F.6B.6C); Anti - Thyroliberin (TRH) antibody; Anti
TNT antibody(AB1 and AB2 antiserum); Anti Triadimefon antibody; Antitriazine antibody
(AM1B5.1); Antitriazine antibody (AM5C5.3); Antitriazine antibody (AM5D1.2);
Antitriazine antibody (AM7B2.1); Antitriazine antibody (SA5A1.1); AntiTriazine serum
(antiametryne); AntiTriazine serum (antiatrazine); AntiTriazine serum (antisimazine);
AntiTriazine serum (antisimetryne); Anti Trifluralin antibody; Anti Trifluralin antibody;
Anti Vincristine antibody; AntiZearalenone antibody; Anti Zeatin riboside antibody; E2 G2
and E4 C2; Fab Fragment K411B derived from MAb K4E7 (isotype IgG2b with k light
chain); LIB-BFNP23 Mab; MAb′s H-7 and H-9 (against O,O-diethyl OP pestides); MoAb
33A7-1-1; MoAb 33B8-1-1; MoAb 33C3-1-1; MoAb 3C10-1-1 and MoAb 3E17-1-1;
MoAb 45D6-5-1; MoAb 45E6-1-1; MoAb 45-1-1; Mutant (GlnL89Glu) in Fab Fragment
K411B derived from MAb K4E7 (isotype IgG2b with k light chain); Mutant (GlnL89Glu/
ValH37Ile/GluL3Val)in Fab Fragment K411B derived from MAb K4E7 (isotype IgG2b
with k light chain); Mutant (GlnL89Glu/ValH37Ile/GluL3Val) in Fab Fragment K411B
derived from MAb K4E7 (isotype IgG2b with k light chain); Mutant (GlnL89Glu/
ValH37Ile)in Fab Fragment K411B derived from MAb K4E7 (isotype IgG2b with k light
chain); Mutant (GlnL89Glu/ValH37Ile) in Fab Fragment K411B derived from MAb K4E7
(isotype IgG2b with k light chain); Mutant (GluH50Gln) in Fab Fragment K411B derived
from MAb K4E7 (isotype IgG2b with k light chain); Mutant (GluH50X) in Fab Fragment
K411B derived from MAb K4E7 (isotype IgG2b with k light chain); Mutant (GlyH100aAla)
in Fab Fragment K411B derived from MAb K4E7 (isotype IgG2b with k light chain);
Mutant (GlyH100aSer) in Fab Fragment K411B derived from MAb K4E7 (isotype IgG2b
with k light chain); Mutant (HisH95Phe) in Fab Fragment K411B derived from MAb K4E7
(isotype IgG2b with k light chain); Mutant (HisH95Tyr) in Fab Fragment K411B derived
from MAb K4E7 (isotype IgG2b with k light chain); Mutant (PheL32Leu) in Fab Fragment
K411B derived from MAb K4E7 (isotype IgG2b with k light chain); Mutant
(TrpH33Phe, Tyr, Leu) in Fab Fragment K411B derived from MAb K4E7 (isotype IgG2b
with k light chain); Mutant (Try196Phe) in Fab Fragment K411B derived from MAb K4E7
(isotype IgG2b with k light chain); Mutant (TryL96Phe) in Fab Fragment K411B derived
from MAb K4E7 (isotype IgG2b with k light chain); Mutant (ValH37Ile) in Fab Fragment
K411B derived from MAb K4E7 (isotype IgG2b with k light chain); Mutant (ValH37Ile)in
Fab Fragment K411B derived from MAb K4E7 (isotype IgG2b with k light chain); P6A7
MAb; PNAS2 6/3 56(1)-1 -5 -1; PNAS2 6/3 56(1)-1 -5 -2; PNAS2 6/3 56(1)-1 -10 -4;
PNAS2 6/3 56(1)-1 -10 -5 and PNAS2 6/3 56(1)-3 -1 -5; Alexa Fluor 405/Cascade Blue
dye antibody; Alexa Fluor 488 dye antibody; BODIPY FL dye antibody; Dansyl antibody;
Fluorescein/Oregon Green dye antibody; Lucifer yellow dye antibody; Tetramethylrhodamine
and Rhodamine Red dye antibody; Texas Red and Texas Red-X dye antibody; Biotin antibody;
Dinitrophenyl antibody or Nitrotyrosine antibody or any binding fragment or CDR from the
aforementioned antibodies or any combination of the aforementioned antibodies,
binding fragments thereof, or CDR domains thereof.

A “marker sequence,” as described herein, encodes a protein that is used for selecting or tracking a protein or cell that has a protein of interest. In the alternatives described herein, the fusion protein provided can comprise a marker sequence that can be selected in experiments, such as flow cytometry. In some alternatives, the marker is the protein Her2tG, CD19t, or EGFRt.

“ScFv” as described herein, is a fusion protein of the variable regions of the heavy (VH) and light chains (VL) of immunoglobulins, connected with a short linker peptide of ten to 25 amino acids or about 25 amino acids. In some alternatives, a CAR is provided, wherein the CAR comprises a ScFv specific for a cell surface tumor molecule or a hapten presented on a cell.

A “ribosome skip sequence” as described herein refers to a sequence that during translation, forces the ribosome to “skip” the ribosome skip sequence and translate the region after the ribosome skip sequence without formation of a peptide bond. Several viruses, for example, have ribosome skip sequences that allow sequential translation of several proteins on a single nucleic acid without having the proteins linked via a peptide bond. As described herein, this is the “linker” sequence. In some alternatives of the nucleic acids provided herein, the nucleic acids comprise a ribosome skip sequence between the sequence for the chimeric antigen receptor and the sequence of the marker protein, such that the proteins are co-expressed and not linked by a peptide bond. In some alternatives, the ribosome skip sequence is a P2A, T2A, E2A or F2A sequence. In some alternatives, the ribosome skip sequence is a T2A sequence. In some alternatives, there are ribosome skip sequences between the two chimeric antigen receptors and a second ribosome skip sequence between one of the chimeric antigen receptors and the marker.

“Biotin” has its plain and ordinary meaning when read in light of the specification, and may include but is not limited to, for example, a water-soluble B-vitamin. In the alternatives herein, the hapten is biotin.

“Fluorescein” has its plain and ordinary meaning when read in light of the specification, and may include but is not limited to, for example, a synthetic organic compound that is soluble in water and alcohol. It is widely used as a fluorescent tracer for many applications. In some alternatives herein, fluorescein is a target moiety on a lipid that is recognized and bound by a chimeric antigen receptor. In some alternatives, the hapten is a fluorescein or derivatives thereof. In some alternatives, the lipid is a phospholipid, such as a phospholipid ether.

As used herein, 2,4-Dinitrophenol (2,4-DNP or simply DNP) is an organic compound with the formula HOC₆H₃(NO₂)₂ and has its plain and ordinary meaning when read in light of the specification. DNP is used as an antiseptic, non-selective bioaccumulating pesticide, herbicide, among others. It is a chemical intermediate in the production of sulfur dyes, wood preservatives, and picric acid. In some alternatives herein, DNP is a target moiety on a lipid that is recognized and bound by a chimeric antigen receptor. In some alternatives, the hapten is DNP or derivatives thereof. In some alternatives, the lipid is a phospholipid, such as a phospholipid ether.

As used herein, “lipid” has its plain and ordinary meaning when read in light of the specification, and may include but is not limited to, for example, a class of organic compounds that comprise carbon chains, fatty acids or a fatty acid derivative that is typically insoluble in water but can integrate into or mix with hydrophobic or organic solvents. Without being limiting, lipids can include fats, waxes, fat soluble vitamins, monoglycerides, diglycerides, triglycerides, sphingolipids, cerebrosides, ceramides, or phospholipids. As described herein are amphiphilic lipids that can have a polar head group and a hydrophobic moiety or hydrophobic group. “Hydrophobic group” or hydrophobic moiety has their plain and ordinary meaning when read in light of the specification, and may include but is not limited to, for example, a molecule or a part of a molecule that is repelled from a mass of water and tends to be non-polar. This can include alkanes, oils or fats. Without being limiting, lipids can be glycerolipids, glycerophospholipids, sphingolipids, sterol lipids, prenol lipids, saccharolipids or polyketides.

In some alternatives, the lipid can be a sphingolipid. The sphingolipid can contain a backbone of sphingoid bases, such as a set of aliphatic amino alcohols that includes sphingosine. A sphingolipid with an R group consisting of a hydrogen atom only is a ceramide. Other common R groups include phosphocholine, yielding a sphingomyelin, and various sugar monomers or dimers, yielding cerebrosides and globosides, respectively. Cerebrosides and globosides are collectively known as glycosphingolipids. In some alternatives, the lipid is a glycosphingolipid.

As provided herein, the lipid comprises a polar head group and a hydrophobic group. In some alternatives, the hydrophobic group comprises a fatty acid such as an aliphatic chain. In some alternatives, the fatty acid is saturated or unsaturated. In some alternatives, the hydrophobic group comprises an alkyl, alkenyl or alkynyl group. In some alternatives, the hydrophobic group comprises a terpenoid lipid such as a steroid or cholesterol. In some alternatives, the hydrophobic group comprises an ether linkage, wherein the ether linkage is between the polar head group and the aliphatic chain. In some alternatives, the lipid is a phospholipid ether. In some alternatives, the polar head comprises a choline, a phosphatidylcholine, sphingomyelin, phosphoethanolamine group, an oligosaccharide residue, a sugar residue, phosphatidyl serine or phosphatidyl inositol. In some alternatives, the sugar is a glycerol.

In some alternatives, the lipid is a single chain alkylphospholipid.

In some alternatives, the lipids comprise a structure of synthetic alkylphospholipids such as edelfosine, perifosine or erucylphosphocholine. In some alternatives, the lipid is a lysophosphatidylcholine, edlfosine, erucylphosphocholine, D-21805 or perfisone. Such lipids are described for example, in van der Lui et al (“A new class of anticancer alkylphospholipids uses lipid rafts as membrane gateways to induce apoptosis in lymphoma cells” Mol Cancer Ther 2007; 6(8), 2007; incorporated by reference in its entirety). In some alternatives of the lipids described herein, a choline within the polar head group can be substituted with a piperidine moiety. In some alternatives, the lipid is an anticancer alkylphospholipid. Anticancer phospholipids are described by vander Lui et al. (“A new class of anticancer alkylphospholipids uses lipid rafts as membrane gateways to induce apoptosis in lymphoma cells” Mol Cancer Ther 2007; 6(8), 2007; incorporated by reference in its entirety).

In some alternatives, the lipids provided herein are synthetic and structurally related antitumor agents that interact with a cell membrane. These types of synthetic lipids are alkylphospholipids and are described by e.g., van Blitterswijk et al. (“Anticancer mechanisms and clinical application of alkylphopholipids” Biochimica et Biophysica Acta 1831 (2013)663-674; herein expressly incorporated by reference in its entirety). Without being limiting, the synthetic alkylphospholipids can include edelfosine, miltefosine, perifosine, erucylphosphocholine or Erufosine. In some alternatives, the lipid is edelfosine, miltefosine, perifosine, erucylphosphocholine or Erufosine. In some alternatives, the lipid is a stable analog of lysophosphatidylcholine. In some alternatives, the lipid is a thio-ether variant of edelfosine, or 1-hexadecylthio-2-methoxymethyl-rac-glycero-3-phosphocholine. In some alternatives, the lipid is LysoPC, edelfosine, Ilmofosine, Miltefosine, Perifosine, Erucylphophocholine, or Erufosine.

As used herein, “polar-head group” has its plain and ordinary meaning when read in light of the specification, and may include but is not limited to, for example, the hydrophilic group of a lipid, such as a phospholipid. “Phospholipids” has its plain and ordinary meaning when read in light of the specification, and may include but is not limited to, for example, a specific class of lipids that can form lipid bilayers due to their amphiphilic characteristic. The phospholipid molecule comprises at least one hydrophobic fatty acid “tail” and a hydrophilic “head” or “polar-head group.” In the alternative herein, the phospholipid or phospholipid ether comprises a polar-head group. In some alternatives, the polar-head group comprises phosphocholine, a piperidine moiety or a trimethylarseno-ethyl-phosphate moiety. In some alternatives, the lipid comprises a target moiety and the CAR is joined to said lipid through an interaction with said target moiety. In some alternatives, the lipid comprises a polar-head group (e.g., comprising an aromatic ring) and a carbon alkyl chain. In some alternatives herein, a complex is provided, wherein the complex comprises a lipid. In some alternatives, the lipid comprises a polar head group. In some alternatives, the lipid is a phospholipid ether. In some alternatives, the phospholipid ether comprises a polar-head group and a carbon alkyl chain. In some alternatives the polar head group comprises a choline, a phosphatidylcholine, sphingomyelin, phosphoethanolamine group, an oligosaccharide residue, a sugar residue, phosphatidyl serine or phosphatidyl inositol. In some alternatives, the polar head group comprises phosphocholine, a piperidine moiety or a trimethylarseno-ethyl-phosphate moiety. In some alternatives, the lipid is a phospholipid ether. In some alternatives, the sugar is a glycerol. In some alternatives, the polar head group comprises a sugar group. In some alternatives, the lipid comprises a mannose-containing head group. In some alternatives, the polar head group comprises sphingosine. In some alternatives, the polar head group comprises a glucose. In some alternatives, the polar head group comprises a di-, tri- or tetra-saccharide. In some alternatives, the lipid is a glucosylcerebroside. In some alternatives, the lipid is a lactosylceramide. In some alternatives, the lipid is a glycolipid. In some alternatives, the glycolipid comprises sugar units such as n-glucose, n-galactose or N-actyl-n-glactosamine. In some alternatives, the lipid comprises a hydrocarbon ring such as a sterol.

In some alternatives, the polar head group of the lipid comprises glycerol. In some alternatives, the polar head group of the lipid comprises a phosphate group. In some alternatives, the polar head group of the lipid comprises choline. In some alternatives, the lipid is a phosphatidylethanolomine. In some alternatives, the lipid is a phosphatidylinositol. In some alternatives, the lipid comprises a sphingoid base backbone. In some alternatives, the lipid comprises a sterol lipid, such as cholesterol or its derivatives. In some alternatives, the lipid comprises saccharolipids. In some alternatives, the polar head group comprises choline, phosphate or glycerol.

In some alternatives, the lipid is a glycolipid. In some alternatives, the lipid comprises a sugar. In some alternatives, the lipid is derived from sphingosine. In some alternatives, the lipid is a glycerol-glycolipid or a sphingo-glycolipid.

In some alternatives, the lipid is an ether lipid with branched hydrophobic chains.

As used herein, “phospholipid ether” has its plain and ordinary meaning when read in light of the specification, and may include but is not limited to, for example, a lipid in which one or more of the carbon atoms on a polar head group are bonded to an alkyl chain via an ether linkage as opposed to the more common ester linkage. In some alternatives, the polar head group is a glycerol.

As used herein, an “antibody” has its plain and ordinary meaning, and in view of the specification, may refer to a large Y-shape protein produced by plasma cells that is used by the immune system to identify and neutralize foreign objects such as bacteria and viruses. The antibody protein can comprise four polypeptide chains; two identical heavy chains and two identical light chains connected by disulfide bonds. Each chain is composed of structural domains called immunoglobulin domains. These domains can contain 70-110 amino acids and are classified into different categories according to their size and function. In some alternatives, CDR regions are found within antibody regions as numbered by Kabat as follows: for the light chain; CDRL1 amino acids 24-34; CDRL2 amino acids 50-56; CDRL3 at amino acids 89-97; for the heavy chain at CDRH1 at amino acids 31-35; CDRH2 at amino acids 50-65; and for CDRH3 at amino acids 95-102. CDR regions in antibodies can be readily determined.

Examples of an antibody or binding fragment thereof, which can be conjugated with target moieties, include monoclonal antibodies, bispecific antibodies, Fab, Fab2, Fab3, scFv, Bis-scFv, minibody, triabody, diabody, tetrabody, VhH domain, V-NAR domain, IgNAR, and camel Ig. Additional examples of an antibody are IgG (e.g., IgG1, IgG2, IgG3, or IgG4), IgM, IgE, IgD, and IgA. Non-limiting examples of antibodies include human antibodies, humanized antibodies, or chimeric antibodies. Non-limiting examples of recombinant antibodies include antibodies that specifically bind to NGF.

An antibody or binding fragment thereof may be specific for a target moiety, and may include, for example, an antigen on a tumor or a hapten. Examples of haptens useful with embodiments provided herein are listed in TABLE 1.

Any of the cancer specific antibodies described herein may bind an antigen on a cancer cell, for example on a tumor cell. Specific tumor cell antigens to which antibodies can be generated, which can be conjugated with target moieties, may include, for example, angiopoietins, transmembrane receptors, cell adhesion molecules, cluster of differentiation molecules, gangliosides, glycoproteins, growth factors, integrins, interleukins, Notch receptors, transmembrane glycoproteins, tumor necrosis factors, or tyrosine kinases. In some embodiments, a tumor cell antigen may include, for example, 5T4, B7-H3, carbonic anhydrase IX, carcinoembryonic antigen, CA-125, CD-3, CD-19, CD-20, CD-22, CD-30, CD-33, CD-38, CD-40, CD-51, CD-52, CD-56, CD-70, CD-74, CD-79b, CD-138, CD-221, CD-319, CD-326, cell adhesion molecule 5, CTLA-4, cytokeratin polypeptides, death receptor 2, DLL4, EGFL7, EGFR, endosialin, EpCAM, FAP, FR-alpha, fibronectin, frizzled receptors, GD2, GPNMB, HER-1, HER-2, HER-3, IGF-IR, IGLF2, LOXL2, mesothelin, MS4A1, mucin SAC, MUC1, Nectin-4, neuropilin, N-glycolil GM3, PSMA, SLAMF7, TAG-72, TRAIL, TYRP1, VEGF, or other cancer expressing antigens. Also contemplated are antibodies that may bind a hapten target moiety. Examples of haptens useful with embodiments provided herein are listed in TABLE 1.

Several types of “spacers” are contemplated for use with embodiments described herein. The spacer for a chimeric antigen receptor refers to a polypeptide spacer, wherein the length of the spacer is selected to modulate e.g., increase or improve the ability of the chimeric antigen receptor to bind its target. The lipid can also comprise a spacer that separates the target moiety from the lipid and is bound to the polar-head group of the lipid. Selected polypeptide spacers for use with chimeric antigen receptors may be screened so as to identify a specific spacer, which is oriented in a manner that promotes a desired binding characteristic e.g., avidity to a target moiety (e.g., a desired receptor interaction or a desired avidity with the receptor). Regarding the spacers that are specific for a lipid, the spacer of the lipid can comprise a PEG spacer, a Hapten spacer, a small peptide or an alkane chain. In some alternatives, the hapten spacer comprises two haptens and is referred to as a hapten (2×) spacer. In some alternatives, the lipid comprises a hydrophobic group, such as an alkane chain. In some alternatives, the alkane chain can comprise 2, 3, 4, 5, 6, 7, 8, 9, 10, 11, 12, 13, 14, 15, 16, 17, or 18 carbons, or any number of carbons in between a range defined by any two aforementioned values. In some alternatives, the PEG spacer comprises 1, 2, 3, 4, 5, 6, 7, 8, 9, 10, 11, 12, 13, 14, 15 or 16 PEG molecules, or any amount of PEG molecules that is within a range defined by any two aforementioned values.

“Cytotoxic T lymphocyte” (CTL), as used herein, refers to a T lymphocyte that expresses CD8 on the surface thereof (e.g., a CD8⁺ T-cell). In some alternatives, such cells are preferably “memory” T-cells (T_{M} cells) that are antigen-experienced. In some alternatives, a cell for fusion protein secretion is provided. In some alternatives, the cell is a cytotoxic T lymphocyte. “Central memory” T-cell (or “T_{CM}”) as used herein, refers to an antigen experienced CTL that expresses CD62L, CCR-7 and/or CD45RO on the surface thereof, and does not express or has decreased expression of CD45RA, as compared to naive cells. In some alternatives, a cell for fusion protein secretion is provided. In some alternatives, the cell is a central memory T-cell (T_{CM}). In some alternatives, the central memory cells are positive for expression of CD62L, CCR7, CD28, CD127, CD45RO, and/or CD95, and may have decreased expression of CD54RA, as compared to naïve cells. “Effector memory” T-cell (or “T_{EM}”) as used herein refers to an antigen experienced T-cell that does not express or has decreased expression of CD62L on the surface thereof, as compared to central memory cells, and does not express or has a decreased expression of CD45RA, as compared to naïve cell. In some alternatives, a cell for fusion protein secretion is provided. In some alternatives, the cell is an effector memory T-cell. In some alternatives, effector memory cells are negative for expression of CD62L and/or CCR7, as compared to naïve cells or central memory cells, and may have variable expression of CD28 and/or CD45RA.

“Naïve T-cells” as used herein, refers to a non-antigen experienced T lymphocyte that expresses CD62L and/or CD45RA, and does not express CD45RO−, as compared to central or effector memory cells. In some alternatives, a cell for fusion protein secretion is provided. In some alternatives, the cell is a naïve T-cell. In some alternatives, naïve CD8+T lymphocytes are characterized by the expression of phenotypic markers of naïve T-cells including CD62L, CCR7, CD28, CD127, and/or CD45RA.

“T-cells” or “T lymphocytes” as used herein can be from any mammalian, preferably primate, species, including monkeys, dogs, and humans. In some alternatives the T-cells are allogeneic (from the same species but different donor) as the recipient subject; in some alternatives the T-cells are autologous (the donor and the recipient are the same); in some alternatives the T-cells arc syngeneic (the donor and the recipients are different but are identical twins).

“T cell precursors” as described herein refers to lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8 and are therefore classed as double-negative (CD4⁻CD8⁻) cells. As they progress through their development, they become double-positive thymocytes (CD4⁺CD8⁺), and finally mature to single-positive (CD4⁺CD8⁻ or CD4⁻CD8⁺) thymocytes that are then released from the thymus to peripheral tissues.

As described herein, “CD8 T-cells” or “killer T-cells” are T— lymphocytes that can kill cancer cells, cells that are infected with viruses or cells that are damages. CD8 T-cells recognize specific antigens, or a protein that is capable of stimulating an immune response and is produced by cancer cells or viruses. If the T-cell receptor of the CD8 T— cell recognizes the antigen, the CD8 T-cell can bind to the presented antigen and destroy the cell.

“Central memory T-cell” (T_{CM}) as used herein refers to an antigen experienced CTL that expresses CD62L or CCR-7 and CD45RO on the surface thereof and does not express or has decreased expression of CD45RA as compared to naïve cells. In some alternatives, central memory cells are positive for expression of CD62L, CCR7, CD28, CD127, CD45RO, and/or CD95, and have decreased expression of CD54RA as compared to naïve cells.

“Effector memory” T-cell (or “T_{EM}”) as used herein refers to an antigen experienced T-cell that does not express or has decreased expression of CD62L on the surface thereof as compared to central memory cells, and does not express or has decreased expression of CD45RA as compared to naïve cell. In some alternatives, effector memory cells are negative for expression of CD62L and/or CCR7, as compared to naïve cells or central memory cells, and have variable expression of CD28 and/or CD45RA. “Effector T-cells” (T_{E} cells) as used herein, refers to antigen experienced cytotoxic T lymphocyte cells that do not express or have decreased expression of CD62L, CCR7, and/or CD28, and are positive for granzyme B and/or perforin, as compared to central memory or naïve T-cells. In some alternatives, a cell for fusion protein secretion is provided. In some alternatives, the cell is an effector T-cell. In some alternatives, the cell does not express or have decreased expression of CD62L, CCR7, and/or CD28, and are positive for granzyme B and/or perforin, as compared to central memory or naïve T-cells.

A “leader sequence” as described herein is also known as a signal sequence that can direct a protein to the cell surface. The leader sequence under the context of a CAR, refers to the first sequence of amino acids in a CAR that directs surface expression. This leader sequence, or signal sequence can be required for surface expression of a protein. In some alternatives, the leader sequence comprises a Granulocyte-macrophage colony-stimulating factor signal sequence.

“Hapten presenting cells” (H-APC) has its plain and ordinary meaning when read in light of the specification, and may include but is not limited to, for example, a cell labelled with a hapten. In some embodiments, a hapten is attached to the extracellular surface of a cell. In some embodiments, an H-APCs can be created from healthy cells of a patient, or cells that are compatible with said patient, and are labeled with a hapten. Examples of haptens useful with embodiments provided herein are listed in TABLE 1. There are many ways to label a cell with a hapten, e.g. chemical, peptide, aptamer, lipid, or protein. An example of how to load cells with a hapten comprises incubation with a fluorescein-lipid overnight with cells of interest. One benefit to the use of fluorescein as a hapten is its fluorescence. Therefore, hapten integration can be monitored by the fluorescence of the fluorescein moiety via flow cytometry. Thus, after incubation excess fluorescein-lipid can be removed, a fraction of the cells can be subjected to flow analysis to analyze hapten integration, and the remaining cells can be used for patient infusion. Post patient infusion H-APCs will slowly lose the hapten (metabolized, defused from the surface, etc.) and return to their original healthy cell form if not targeted by a CAR T cell, demonstrating a layer of safety in this approach. In some embodiments, a cell can be transduced to express a hapten on the extracellular surface of the cell. In some embodiments, a hapten can be covalently attached to the extracellular surface of a cell. In some embodiments, a hapten can be covalently attached to the extracellular surface of a cell via a phospholipid, such as a phospholipid ether.

“Stimulation” or activation of T-cells refers to the method of inducing a T-cell to initiate a response, such as a signal transduction response, e.g., proliferation, while preserving T-cell viability and immune function. Stimulation of the cell may also induce that activity of the T cell comprising the CAR. In some alternatives, the stimulating is performed with an antibody-bound support comprising antiCD3 and/or antiCD28 antibodies. In some alternatives, the method further comprises removing the antibody-bound support, such as beads or particles or a substrate such as a dish or tube. As described in the alternatives herein, the T cells comprising the hapten specific CAR may be stimulated using hapten antigen presenting cells (H-APC) or stimulation may be performed ex vivo, using a support such as beads that are conjugated to a hapten, for example.

“Chemotherapeutic drugs” are category of anticancer medicaments that can use, for example, chemical substances, such as anticancer drugs (chemotherapeutic agents) that can be given as part of a standardized chemotherapy regimen. Chemotherapeutic drugs can be given with a curative intent, or it can aim to prolong life or to reduce symptoms (palliative chemotherapy). Additional chemotherapies can also include hormonal therapy and targeted therapy, as it is one of the major categories of medical oncology (pharmacotherapy for cancer). These modalities are often used in conjunction with other cancer therapies, such as radiation therapy, surgery, and/or hyperthermia therapy. In few cases, cancer has been known to spread due to surgery. In some alternatives, a genetically modified immune cell is administered to the tumor site prior to or after a surgical procedure. In some alternatives herein, the subject treated with the CAR T cell therapy are selected to receive chemotherapeutic drugs or anticancer drugs. Some newer anticancer drugs (for example, various monoclonal antibodies, humanized versions thereof and binding fragments thereof) are not indiscriminately cytotoxic, but rather target proteins that are abnormally expressed in cancer cells and that are essential for their growth. Such therapies are often referred to as targeted therapy (as distinct from classic chemotherapy) and are often used alongside traditional chemotherapeutic agents in antineoplastic treatment regimens. In some alternatives, the methods described herein can further comprise administering any one or more of these targeted anticancer therapies (for example, various monoclonal antibodies, humanized versions thereof and/or binding fragments thereof).

Chemotherapy, in which chemotherapeutic drugs are administered, can use one drug at a time (single-agent chemotherapy) or several drugs at once (combination chemotherapy or polychemotherapy). The combination of chemotherapy and radiotherapy is chemoradiotherapy. Chemotherapy using drugs that convert to cytotoxic activity only upon light exposure is called photochemotherapy or photodynamic therapy. In some alternatives of administering the genetically modified immune cell described herein, the method can further comprise administering to a subject having cancer, photochemotherapy or photodynamic therapy after receiving the genetically modified immune cells or genetically engineered macrophages (GEMs).

Chemotherapeutic drugs can include but are not limited to antibody-drug conjugates (for example, an antibody attached to a drug by a linker), nanoparticles (for example a nanoparticle can be 1-1000 nanometer sized particle for promoting tumor selectivity and aid in delivering low-solubility drugs), electochemotherapy, alkylating agents, antimetabolites (for example, 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, and Thioguanine), antitumor antibiotics, topoisomerase inhibitors, mitotic inhibitors, corticosteroids, DNA intercalating agents, or checkpoint inhibitors (for example checkpoint kinases CHK1, or CHK2). In some alternatives of the methods described herein, the genetically modified immune cells comprising CAR or compositions comprising genetically modified immune cells comprising a CAR are administered in combination with one or more anticancer agents, such as any one or more of the foregoing compounds or therapies. In some alternatives, the one or more anticancer agent that is co-administered or administered in conjunction with the genetically modified immune cells, comprises antibody-drug conjugates, nanoparticles, electrochemotherapy, alkylating agents, antimetabolites, antitumor antibiotics, topoisomerase inhibitors, mitotic inhibitors, corticosteroids, DNA intercalating agents, or checkpoint inhibitors. In some alternatives, the antimetabolites comprises 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, or Thioguanine.

“Cancer,” has its plain and ordinary meaning when read in light of the specification, and may include but is not limited to, for example, a group of diseases involving abnormal cell growth with the potential to invade or spread to other parts of the body. Subjects that can be addressed using the methods described herein include subjects identified or selected as having cancer, including but not limited to colon, lung, liver, breast, renal, prostate, ovarian, skin (including melanoma), bone, and/or brain cancer, etc. Such identification and/or selection can be made by clinical or diagnostic evaluation. In some alternatives, the tumor associated antigens or molecules are known, such as melanoma, breast cancer, brain cancer, squamous cell carcinoma, colon cancer, leukemia, myeloma, or prostate cancer or any combination thereof. Examples include but are not limited to B cell lymphoma, breast cancer, brain cancer, prostate cancer, or leukemia. In some alternatives, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia. In some alternatives, a method of treating, ameliorating, or inhibiting a cancer in a subject is provided. In some alternatives, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer. In some alternatives, the subject is selected to receive an additional cancer therapy, which can include a cancer therapeutic, radiation, chemotherapy, or a drug suitable for cancer therapy. In some alternatives, the drugs comprise Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

“Tumor microenvironment” has its plain and ordinary meaning when read in light of the specification, and may include but is not limited to, for example, a cellular environment, wherein a tumor exists. Without being limiting, the tumor microenvironment can include surrounding blood vessels, immune cells, fibroblasts, bone marrow-derived inflammatory cells, lymphocytes, signaling molecules and/or the extracellular matrix (ECM). In some alternatives herein, the T-cells bearing the CAR are administered within a tumor environment and are stimulated using a H-APC.

### DETAILED DESCRIPTION

Some embodiments of the methods and compositions provided herein relate to the use of hapten labeled cells to stimulate chimeric antigen receptor (CAR) T cells. In some embodiments, CAR T cells can include a CAR that specifically binds to a hapten. Some embodiments relate to the in vivo or in vitro stimulation CAR T cells by hapten labeled cells.

Chimeric antigen bearing cells are immune cells that are engineered to direct the immune cells (T-cells) to a biomarker that is associated with the surface of a malignant cell. These surface targets or antigens allow a directed, specific therapy that reduces healthy tissue destruction and preserves the patient's immune system during therapy. T cells are a critical component of the adaptive immune system as they not only orchestrate cytotoxic effects but may provide long term cellular ‘memory’ of specific antigens. T cells endogenously require the interaction between MHC displayed peptides and their TCR to activate, but CAR T cells are engineered to activate via a tumor-associated or tumor-specific antigen (TAA and TSA, respectively). Thus, CAR T cells may be considered as a “living drug” comprising a targeting domain (single chain variable fragment (scFv), peptides, polypeptides, ligands, muteins, spacers, and/or linkers) fused to the signaling domain of a T cell. Upon recognition and binding of the targeting domain to its specific target, the T cell activates and subsequent target cell killing is initiated. CAR T cell therapy has been revolutionary in the treatment of hematological malignancies with the targets CD19 and CD20. However, CAR T cells have been unable to translate effectively and efficiently to solid tumors, and work will need to be done to address this area. Embodiments related to the stimulation of CAR T cells stimulated are provided herein. Additionally, stimulation of the CAR T cells may obviate current challenges facing CAR T cell therapy, such as persistence in vivo and the immunosuppressive tumor microenvironment, which are important for further CAR T cell development and success.

In some of the alternatives herein, T cells are transduced, transfected, or transformed to express at least two unique CARs (dual CAR) in one cell, wherein one CAR is specific to a tumor target and the other CAR is specific to a hapten, e.g. fluorescein. Alternatively, T cells are transduced, transfected, or transformed to express a single CAR that contains two targeting moieties, e.g. two scFvs, (bispecific CAR), wherein one targeting moiety is tumor-specific and the other CAR is specific for a hapten. However, if the tumor is labeled with the hapten then the antiHapten CAR would be the only CAR necessary. Dual and bispecific CAR T cells can be generated by many different methods, e.g. dual transduction with viral vectors, a single transduction with a viral vector where the virus contains both CARs, non-viral transposon vectors, etc. There are many ways to select for pure or isolated CAR T cell populations. For example, using two surface tags, e.g. EGFRt, Her2tG, CD19t, etc., and then sorting the cells by each surface marker. Also, in some alternatives herein, the antihapten CAR is sorted using a substrate, such as magnetic beads, or a dish or tube labeled with hapten. A unique characteristic of this approach is that the antihapten CAR can also be constitutively expressed since it should not recognize any endogenous epitopes in the patient.

The H-APC (hapten antigen presenting cells) are preferably generated from healthy cells of a patient, or cells that are compatible with said patient, and ex vivo labeling of the cells with a hapten. Without being limiting, examples of haptens are fluorescein, urushiol, quinone, or biotin. More examples of haptens useful with embodiments provided herein are listed in TABLE 1. There are many ways to label a cell with a hapten, (e.g. chemical, peptide, aptamer, lipid, or protein). For example, one may incubate a fluorescein-lipid overnight with cells of interest. A benefit to the use of fluorescein as a hapten is its fluorescence. By this approach, hapten integration can be monitored by the fluorescence of the fluorescein moiety via flow cytometry. Thus, after incubation excess fluorescein-lipid can be removed, a fraction of the cells can be subjected to flow analysis to analyze hapten integration, and the remaining cells can be used for patient infusion. Post patient infusion, H-APCs will slowly lose the hapten (metabolized, defused from the surface) and return to their original healthy cell form if not targeted by a CAR T cell, thereby providing a layer of safety in this approach.

H-APCs can be administered at any point during therapy if CAR T cells need to be stimulated in a patient. One example of this need is when CAR T cells contract and lose potency once hematologic cancers reach final stages of regression due to low cancer cell levels. In this case, H-APC are infused to expand and activate the CAR T cells to continue the regression of the cancer and hopefully effect complete tumor remission.

Another example of this need is during solid tumor therapy. Solid tumors are often very immuno-suppressive and the addition of H-APC may help stimulate T cells to overcome the immunosuppressive tumor environment. The H-APC approach offers a safe way to stimulate CAR T cells in vivo.

H-APC can also be used to stimulate CAR T cells in vitro. Under certain clinical protocols, magnetic beads are used to stimulate CAR T cells through the TCR before they are infused back into the patient. H-APC could be made using magnetic beads labeled with the hapten. In this case the H-APC would stimulate the cells through the CAR prior to infusion.

Additionally, if a rapid expansion protocol (REP) prior to infusion back into a patient is desired, H-APC is a safe alternative. Standard REP uses irradiated TM-LCL and PBMC as feeder cells. As an alternative, there are multiple ways HAPCs can be utilized in a REP. First, if H-APCs were made from the patient's own cells the irradiation step could be skipped, and the culturing of TM-LCL's and isolation of PBMC would be unnecessary. Second, H-APC could be generated by irradiated cells from another donor. Finally, this sort of H-APC REP can be used for laboratory work instead of a standard REP. These examples provide several approaches to selectively expand CAR T cells via hapten-specific stimulation.

The clinical hurdles faced by CAR T cell therapy, especially for solid tumors, may necessitate the use of supplemental support beyond the activity of a single CAR. H-APC provide a mechanism to improve CAR T cell engraftment and persistence beyond what is demonstrated in current clinical protocols and may promote T cell migration to immunosuppressive tumor metastasis sites of solid tumors. Whereas in hematologic cancers the threshold of cancer can be too low to allow for primary CAR T cell engraftment, and the H-APC can be used to promote primary activation. In both cases the antiHapten CAR will drive the activation, proliferation and dispersion of the infused CAR T cells while the other expressed CAR will orchestrate the ablation of the tumor. This strategy also offers a unique way to REP CAR T cells before infusion into a patient.

The alternatives described herein aim to improve the curative properties of CAR T cell therapy in both solid and hematologic cancers. H-APC provide the potential to stimulate CAR T cells in vivo to overcome the immunosuppressive tumor microenvironment, help expand the ability of CAR T cells to find and eradicate trace amounts of cancer or simply to help support the CAR T cells. The H-APC can be safe to use and H-APC not lysed by CAR T cells will have the hapten safely degraded overtime and will return to a normal healthy cell. Additionally, using REP with a H-APC to stimulate cells would have the benefits of lower costs and shorter periods for cell culturing.

Another factor to consider regarding manufacturing a cell with two CARs in one viral vector is pushing the size limit. In some alternatives herein, co— transduction of additional vectors made be performed. Alternatives to manufacturing the CAR T cells to avoid potential size limitation are contemplated, as well.

The toxicity of a hapten is also considered. However, one of skill in the art would appreciate that assays may be performed to determine if a selected hapten may be well tolerated by people, e.g. fluorescein. The toxicity of the binding component conjugated to the hapten (e.g. lipid, protein, peptide, or aptamer) could be a problem too. Again, control is present because a binding component can be selected, which is metabolized or passed through the body rapidly. PCT/US2018/017126 describes one such chemical (herein expressly incorporated by reference in its entirety).

Similarly, autologous T cells transfected to express cell-surface tROR 1 (ROR1+T-APC) was previously developed (Berger et al. 2015, Cancer Immunology Research, 3(2), 206-216). However, a major difference between Berger and the alternatives described herein is that Berger et al. had to transduce, expand, culture and characterize their ROR1+T-APC product, which is on the time scale of weeks to months and has a high cost associated with it. In the alternatives described herein, cells only need to be loaded with hapten, which can be performed in a very short time scale (e.g. hours) and then can be infused back into the patient. The type of cell can be varied, as well, in the system of the alternatives described herein. Therefore, precious T cells would not need to be used. Additionally, the technique described by Berger has not been used in context with a solid tumor. In addition, Berger genetically engineered a T-APC, which requires substantial costs and time. In contrast, embodiments provided herein provide hapten labeled cells quickly and efficiently by direct attachment to the extracellular surface of a cell. As such, the methods described in the alternatives herein will revolutionize the field of solid tumor T cell immunotherapy and greatly improve the hematologic cancer therapy with CAR T cells that are currently existing.

Some embodiments of the methods and compositions provided herein include aspects disclosed in WO 2018/148224; WO 2019/156795; WO 2019/144095; U.S. 2019/0224237; and PCT/US2019/044981 published as WO 2020/033272, which are all each expressly incorporated herein by reference in its entirety.

### Inducing Expansion of CAR T Cells

Some embodiments of the methods and composition provided herein include inducing expansion of a chimeric antigen receptor (CAR) T cell. In some such embodiments, a CAR T cell is incubated with a hapten antigen presenting cell (H-APC) under conditions, which the induce expansion of the CAR T cell. In some embodiments, a CAR of the CAR T cell specifically binds to a hapten attached to the H-APC. Some embodiments include treating, inhibiting, or ameliorating a cancer in a subject. In some embodiments, a subject is administered an effective amount of CAR T cell, in which the CAR of the CAR T cell specifically binds to a tumor specific antigen of the cancer, and inducing expansion of the CAR T cell by incubating the CAR T cell with a hapten antigen presenting cell (H-APC), wherein a CAR of the CAR T cell specifically binds to a hapten attached to the H-APC. In some embodiments, the CAR T cell and the H-APC are derived from a single subject, such as a human. In some embodiments, the subject is mammalian, such as human, livestock animal, or domestic animal.

In some embodiments, the CAR T-cell can include a bispecific CAR. For example, a CAR can have two specific binding domains, a first binding domain that can specifically bind a target, such as a tumor specific antigen; and a second binding domain that can specifically bind to the hapten.

In some embodiments, the CAR T-cell can include more than one CAR. For example, a CAR can include a first CAR that includes a first binding domain that can specifically bind a target, such as a tumor specific antigen; and a second CAR that includes a second binding domain that can specifically bind to the hapten.

In some embodiments, the CAR T-cell can include a CAR that can bind a target, such as a tumor specific antigen, and can also bind a hapten. In some such embodiments, the target and the hapten can comprise the same binding moiety, or substantially the same binding moiety, such that the CAR can bind the binding moiety of the target, and the binding moiety of the hapten. In some such embodiments, a target and a hapten can be a tumor antigen provided herein.

Examples of target antigens that can be used with embodiments provided herein include CD19, CD22, HER2, CD7, CD30, B cell maturation antigen (BCMA), GD2, glypican-3, MUC1, CD70, CD33, epithelial cell adhesion molecule (EpCAM), Epidermal Growth Factor variant III, receptor tyrosine kinase-like orphan receptor 1 (ROR1), CD123, Prostate Stem Cell Antigen (PSCA), CD5, Lewis Y antigen, B7H3, CD20, CD43, HSP90, or IL13 or any combination thereof.

Examples of haptens that can be used with embodiments provided herein include those haptens listed in TABLE 1. In some embodiments, haptens useful with embodiments provided herein include fluorescein, urushiol, quinone, biotin, or dinitrophenol, and/or derivatives thereof.

In some embodiments, a hapten is covalently attached to the extracellular surface of a cell to prepare a H-APC. In some embodiments, the hapten is attached to the H-APC via a phospholipid ether (PLE).

In some embodiments, the incubation can be in vitro. For example, CAR T cells can be prepared by transducing cells with a vector encoding a CAR, and the transduced cells can be induced to expand by incubating the cells with a H-APC. In some embodiments, the expanded cells can be administered to a subject, such as a human. In some embodiments, the incubation can be in vivo. For example, a subject can be administered CAR T cells. The subject can also be administered H-APC which induce expansion of the CAR T cells in vivo.

In some embodiments, the CAR T cell is derived from a CD4+ cell or a CD8+ cell. In some embodiments, the CD8+ cell is a CD8+T cytotoxic lymphocyte cell selected from the group consisting of naïve CD8+ T cells, central memory CD8+ T cells, effector memory CD8+ T cells, and bulk CD8+ T cells. In some embodiments, the CD8+ cell is a CD8+ cytotoxic T lymphocyte cell is a central memory T cell and, wherein the central memory T cell is positive for CD45RO+, CD62L+, and CD8+. In some embodiments, the CD4+ cell is a CD4+T helper lymphocyte cell selected from the group consisting of naïve CD4+ T cells, central memory CD4+ T cells, effector memory CD4+ T cells, and bulk CD4+ T cells. In some embodiments, the CD4+ helper lymphocyte cell is a naïve CD4+ T cell and, wherein the naïve CD4+ T cell is positive for CD45RA+, CD62L+ and CD4+ and negative for CD45RO. In some embodiments, the CAR T cell is derived from a precursor T cell. In some embodiments, CAR T cell is derived from hematopoietic stem cell. In some embodiments, the H-APC is derived from a healthy cell of a subject, such as a T cell, and a B cell.

In some embodiments, the healthy cells can be T cells, B cells, monocyte, macrophages, dendric cells, NK cells, or red blood cells. In some embodiments, the healthy cells can be any peripheral blood mononuclear cells. In some embodiments, the healthy cells can be any healthy cells from the the body. In some embodiments, the healthy cells can be any cells from an apheresis product. In some embodiments, the healthy cells can be any cells that can be labeled ex vivo.

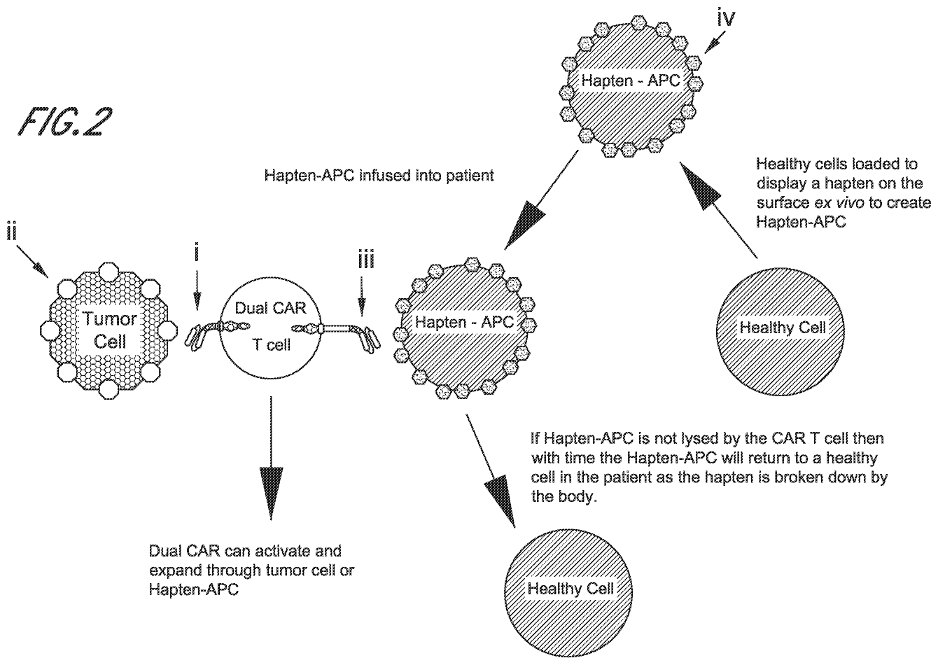

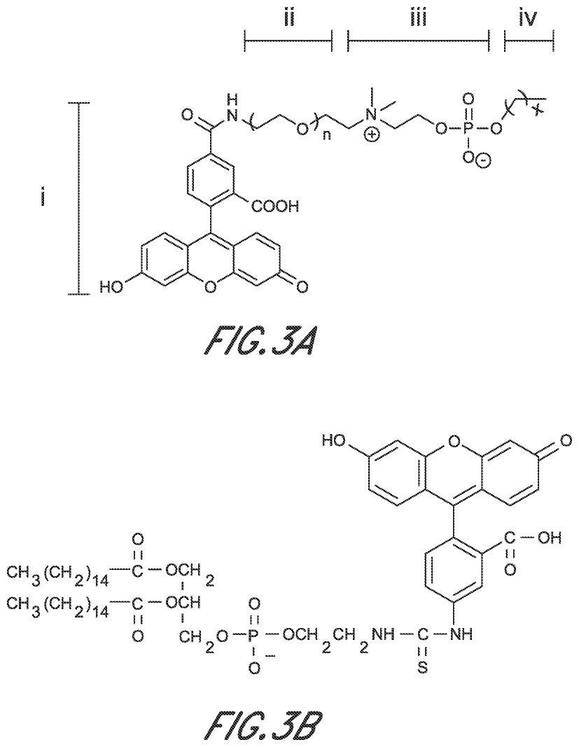

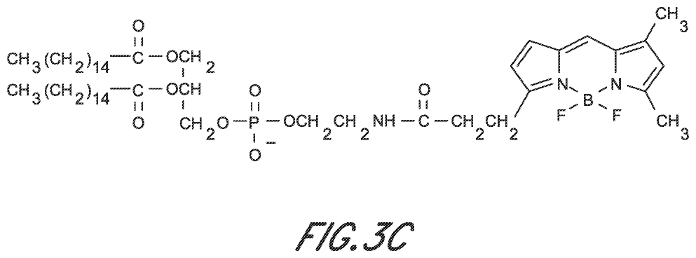

In some embodiments, a single CAR cell is used with H-APC. In some embodiments, a multimeric CAR is used with H-APC. In some embodiments, a single antihapten CAR T cell is used, e.g., when a tumor is labeled with a hapten (for example, CD19 antibody labeled with hapten, hapten-PLE, small molecule labeled with hapten, peptide labeled with hapten, aptamer labeled hapten, or other hapten-labeled tumor cells) and the H-APC is made with the same hapten to expand the antihapten CAR T cells in a patient. In some embodiments, a dual or bispecific CAR cell can be used, where one CAR (e.g., CD19, CD22, or ROR1) attacks the cancer and the antihapten CAR is used to expand the dual or bispecific CAR via H-APC (See, for example, FIG. 2). In some embodiments, this can be extended further, where more than two CARs plus an antihapten CAR are loaded into a cell (for example, CD19 and CD22 for fighting ALL) and the antihapten CAR is used to activate and expanded the CAR T cells.

In some alternatives, the T cell is a non-autologous T cell.

In some alternatives, the methods disclosed herein can be used to expand any cell via a CAR and H-APC. For example, a B cell expressing an anti-hapten CAR could use to expand those cells with H-APC. As such this approach could be used to expand any type of cells in vivo.

In some alternatives, CAR T cells can be generated not just as a therapy for a cancer but also as a therapy for viral infections (e.g., HIV or hepatitis) as well as, CAR T cells that can be a therapy for autoimmune diseases and conditions associated therewith.

In some alternatives, tumor infiltrating lymphocytes (TILs) can be collected from a tumor/cancer, transduced with CAR, and expanded in vitrolin vivo using H-APC.

### Nucleic Acids Encoding CARs and Bispecific CARs

In some alternatives, one or more nucleic acids for the expression of a first chimeric antigen receptor and a second chimeric antigen receptor is provided. The nucleic acid or nucleic acids may be provided within a single vector or within a plurality of vectors in order to accommodate the payload size of two CARs. The one or more nucleic acids may comprise a first sequence encoding the first chimeric antigen receptor, wherein the first chimeric antigen receptor comprises a first ligand binding domain, which is specific for a tumor antigen, a first polypeptide spacer, a first transmembrane domain and a first intracellular signaling domain; and a second sequence encoding the second chimeric antigen receptor, wherein the second chimeric antigen receptor comprises a second ligand binding domain specific for a hapten, a second polypeptide spacer, a second transmembrane domain and a second intracellular signaling domain. In some alternatives the first ligand binding domain is specific for a tumor cell antigen. In some alternatives, the tumor cell antigen comprises 5T4, B7-H3, carbonic anhydrase IX, carcinoembryonic antigen, CA-125, CD-3, CD-19, CD-20, CD-22, CD-30, CD-33, CD-38, CD-40, CD-51, CD-52, CD-56, CD-70, CD-74, CD-79b, CD-138, CD-221, CD-319, CD-326, cell adhesion molecule 5, CTLA-4, cytokeratin polypeptides, death receptor 2, DLL4, EGFL7, EGFR, endosialin, EpCAM, FAP, FR-alpha, fibronectin, frizzled receptors, GD2, GPNMB, HER-1, HER-2, HER-3, IGF-IR, IGLF2, LOXL2, mesothelin, MS4A1, mucin SAC, MUC1, Nectin-4, neuropilin, N-glycolil GM3, PSMA, SLAMF7, TAG-72, TRAIL, TYRP1 or VEGF. In some embodiments, the CAR can specifically bind to a hapten listed in TABLE 1. In some embodiments, the hapten can be selected from fluorescein, urushiol, quinone, biotin, or dinitrophenol, or a derivative thereof. In some alternatives, the first and/or second ligand binding domain comprises an antibody or binding fragment thereof or scFv. In some alternatives, the second ligand binding domain comprises a binding fragment of an antibody such as an antibody directed against a hapten listed in TABLE 1, or an antibody listed in TABLE 2. Example amino acid sequences and nucleic acids encoding antigen binding domains, such as svFc, that can bind haptens, such as fluorescein or dinitrophenol, are listed in TABLE 3, all of which can be incorporated into one or more of the embodiments described herein.

TABLE 3

| SEQ ID NO: | Sequence |
|---|---|
| SEQ ID NO: 01 | SVLTQPSSVSAAPGQKVTISCSGSTSNIGNNYVSWYQQHPGKAPKLMIYDVSK |
| FITCE2 scFv | RPSGVPDRFSGSKSGNSASLDISGLQSEDEADYYCAAWDDSLSEFLFGTGTKLT |
| | VLGGGGGSGGGGSGGGGSQVQLVESGGNLVQPGGSLRLSCAASGFTFGSFS |
| | MSWVRQAPGGGLEWVAGLSARSSLTHYADSVKGRFTISRDNAKNSVYLQM |
| | NSLRVEDTAVYYCARRSYDSSGYWGHFYSYMDVWGQGTLVTVS |
| SEQ ID NO: 02 | SVLTQPSSVSAAPGQKVTISCSGSTSNIGNNYVSWYQQHPGKAPKLMIYDVSK |
| FITCE2 TyrH133Ala | RPSGVPDRFSGSKSGNSASLDISGLQSEDEADYYCAAWDDSLSEFLFGTGTKLT |
| scFv | VLGGGGGSGGGGSGGGGSQVQLVESGGNLVQPGGSLRLSCAASGFTFGSFS |
| | MSWVRQAPGGGLEWVAGLSARSSLTHYADSVKGRFTISRDNAKNSVYLQM |
| | NSLRVEDTAVYYCARRSYDSSGYWGHFASYMDVWGQGTLVTVS |
| SEQ ID NO: 03 | VLTQPSSVSAAPGQKVTISCSGSTSNIGNNYVSWYQQHPGKAPKLMIYDVSKR |
| FITCE2 HisH131Ala | PSGVPDRFSGSKSGNSASLDISGLQSEDEADYYCAAWDDSLSEFLFGTGTKLTV |
| scFv | LGGGGGSGGGGSGGGGSQVQLVESGGNLVQPGGSLRLSCAASGFTFGSFSM |
| | SWVRQAPGGGLEWVAGLSARSSLTHYADSVKGRFTISRDNAKNSVYLQMNS |
| | LRVEDTAVYYCARRSYDSSGYWGAFYSYMDVWGQGTLVTVS |
| SEQ ID NO: 04 | DVVMTQTPLSLPVSLGDQASISCRSSQSLVHSNGNTYLRWYLQKPGQSPKVLI |
| 4M5.3 scFv | YKVSNRVSGVPDRFSGSGSGTDFTLKINRVEAEDLGVYFCSQSTHVPWTFGGG |
| | TKLEIKSSADDAKKDAAKKDDAKKDDAKKDGGVKLDETGGGLVQPGGAMKL |
| | SCVTSGFTFGHYWMNWVRQSPEKGLEWVAQFRNKPYNYETYYSDSVKGRFT |
| | ISRDDSKSSVYLQMNNLRVEDTGIYYCTGASYGMEYLGQGTSVTVS |
| SEQ ID NO: 05 | DYKDIQMTQSPSSLSASVGDRVTITCRASQSLVHSQGNTYLRWYQQKPGKAP |
| 4D5Flu scFv | KVLIYKVSNRFSGVPSRFSGSGSGTDFTLTISSLQPEDFATYYCQQSTHVPWTF |
| | GQGTKVELKRAGGGGSGGGGSGGGGSSGGGSGGGGSGGGGSEVQLVESG |
| | GGLVQPGGSLRLSCAASGFTFSDYWMNWVRQAPGKGLEWVAQIRNKPYNY |
| | ETYYADSVKGRFTISRDTSKNTVYLQMNSLRAEDTAVYYCTGSYYGMDYWGQ |
| | GTLVTVSS |
| SEQ ID NO: 06 | DVVMTQTPLSLPVSLGDQASISCRSSQSLVHSQGNTYLRWYLQKPGQSPKVLI |
| 4420 | YKVSNRFSGVPDRFSGSGSGTDFTLKISRVEAEDLGVYFCSQSTHVPWTFGGG |
| | TKLEIGGGGSGGGGSGGGGSEVKLDETGGGLVQPGRPMKLSCVASGFTFSDY |
| | WMNWVRQSPEKGLEWVAQIRNKPYNYETYYSDSVKGRFTISRDDSKSSVYLQ |
| | MNNLRVEDMGIYYCTGSYYGMDYWGQGTSVTVSS |
| SEQ ID NO: 07 | cagtgtcagcagctggagcagtccggaggaggagccgaaggaggcctggtcaagcctgggggat |
| anti DNP scFv | ccctggaactctgctgcaaagcctctggattctccctcagtagtagctactgcatatgttgggtccgc |
| (VH to VL from | caggctccagggaaggggctggagtggatcggatgcatttatgctggtagtagtggtagcacttac |
| rabbit) | tacgcgagctgggtgaatggccgattcactctctccagagacattgaccagagcacaggttgccta |
| | caactgaacagtctgacagccgcggacacggccatgtattactgtgcgagagccccctatagtagt |
| | ggctgggtcctctactttaacttgtggggcccaggcaccctggtcattgtctcctcaggcggagggg |
| | gctctggcggcggaggatctgggggagggggcagcccaggtgccacatttgcccaagtgctgacc |
| | cagactccatcgcctgtgtctgcagctgtgggaggcacagtcaccatcagttgccagtccagtgag |
| | agtgtttatggtaacagccgcttagcctggtatcagcagaaaccagggcagtctcccaagctcctg |
| | atctattatgcatccactctggcatctggggtcccttcgcggttcaaaggcagtggatctgggacac |
| | agttcactctcaccattagcgacctggagtgtgacgatgctgcctcttactactgtcaaggcggttat |
| | tatagtggtaatcttgatgcgcttgctttcggcggagggaccgaggtggtggtcagaggt |
| SEQ ID NO: 08 | QCQQLEQSGGGAEGGLVKPGGSLELCCKASGFSLSSSYCICWVRQAPGKGLE |
| anti DNP scFv | WIGCIYAGSSGSTYYASWVNGRFTLSRDIDQSTGCLQLNSLTAADTAMYYCAR |
| (VH to VL from | APYSSGWVLYFNLWGPGTLVIVSSGGGGSGGGGSGGGGSPGATFAQVLTQT |
| rabbit) | PSPVSAAVGGTVTISCQSSESVYGNSRLAWYQQKPGQSPKLLIYYASTLASGVP |
| | SRFKGSGSGTQFTLTISDLECDDAASYYCQGGYYSGNLDALAFGGGTEVVVRG |
| SEQ ID NO: 09 | ccaggtgccacatttgcccaagtgctgacccagactccatcgcctgtgtctgcagctgtgggaggc |
| anti DNP scFv | acagtcaccatcagttgccagtccagtgagagtgtttatggtaacagccgcttagcctggtatcagc |
| (VL to VH from | agaaaccagggcagtctcccaagctcctgatctattatgcatccactctggcatctggggtcccttc |
| rabbit) | gcggttcaaaggcagtggatctgggacacagttcactctcaccattagcgacctggagtgtgacga |
| | tgctgcctcttactactgtcaaggcggttattatagtggtaatcttgatgcgcttgctttcggcggagg |
| | gaccgaggtggtggtcagaggtggcggagggggctctggcggcggaggatctgggggagggggc |
| | agccagtgtcagcagctggagcagtccggaggaggagccgaaggaggcctggtcaagcctgggg |
| | gatccctggaactctgctgcaaagcctctggattctccctcagtagtagctactgcatatgttgggtc |
| | cgccaggctccagggaaggggctggagtggatcggatgcatttatgctggtagtagtggtagcact |
| | tactacgcgagctgggtgaatggccgattcactctctccagagacattgaccagagcacaggttgc |
| | ctacaactgaacagtctgacagccgcggacacggccatgtattactgtgcgagagccccctatagt |
| | agtggctgggtcctctactttaacttgtggggcccaggcaccctggtcattgtctcctca |
| SEQ ID NO: 10 | PGATFAQVLTQTPSPVSAAVGGTVTISCQSSESVYGNSRLAWYQQKPGQSPK |
| anti DNP scFv | LLIYYASTLASGVPSRFKGSGSGTQFTLTISDLECDDAASYYCQGGYYSGNLDAL |
| (VL to VH from | AFGGGTEVVVRGGGGGSGGGGSGGGGSQCQQLEQSGGGAEGGLVKPGGS |
| rabbit) | LELCCKASGFSLSSSYCICWVRQAPGKGLEWIGCIYAGSSGSTYYASWVNGRFT |
| | LSRDIDQSTGCLQLNSLTAADTAMYYCARAPYSSGWVLYFNLWGPGTLVIVSS |

In some alternatives, the first polypeptide spacer or second polypeptide spacer or both comprise a length of 1-24, 25-50, 51-75, 76-100, 101-125, 126-150, 151-175, 176-200, 201-225, 226-250 or 251-275 amino acids. In some alternatives, the nucleic acid further comprises a leader sequence. In some alternatives, the first and/or second intracellular signaling domains comprises CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83 or CD3-zeta cytoplasmic domains or both. In some alternatives, the intracellular signaling domain comprises a portion of CD3 zeta and a portion of 4-1BB. In some alternatives, the nucleic acid further comprises a sequence encoding a marker sequence. In some alternatives, the marker is EGFRt, CD19t, or Her2tG. In some alternatives, the first or second transmembrane domain or both comprises the transmembrane domain of CD28. In some alternatives, the nucleic acid further comprises a sequence encoding a cleavable linker. In some alternatives, the linker is a ribosome skip sequence. In some alternatives, the ribosome skip sequence is P2A, T2A, E2A or F2A. The cleavable linker may be in between the sequences encoding the two chimeric antigen receptors. Additionally, a cleavable linker may be used in between any one of the chimeric antigen receptors and the sequence encoding the marker protein. In some alternatives, one or more vectors comprising the one or more nucleic acids of any one of the alternatives described herein is provided. In some alternatives, the chimeric antigen receptors encoded by the nucleic acids of any one of the alternatives herein or the vector of any one of the alternatives herein is provided.

In some alternatives, one or more nucleic acids for the expression of a first chimeric antigen receptor and a second chimeric antigen receptor are provided and the one or more nucleic acids comprise a first nucleic acid comprising a first sequence encoding the first chimeric antigen receptor, wherein the first chimeric antigen receptor comprises a first ligand binding domain, which is specific for a tumor antigen, a first polypeptide spacer, a first transmembrane domain and a first intracellular signaling domain and a second nucleic acid comprising a second sequence encoding the second chimeric antigen receptor, wherein the second chimeric antigen receptor comprises a second ligand binding domain, which is specific for a hapten, a second polypeptide spacer, a second transmembrane domain and a second intracellular signaling domain. In some alternatives, the first ligand binding domain is specific for 5T4, B7-H3, carbonic anhydrase IX, carcinoembryonic antigen, CA-125, CD-3, CD-19, CD-20, CD-22, CD-30, CD-33, CD-38, CD-40, CD-51, CD-52, CD-56, CD-70, CD-74, CD-79b, CD-138, CD-221, CD-319, CD-326, cell adhesion molecule 5, CTLA-4, cytokeratin polypeptides, death receptor 2, DLL4, EGFL7, EGFR, endosialin, EpCAM, FAP, FR-alpha, fibronectin, frizzled receptors, GD2, GPNMB, HER-1, HER-2, HER-3, IGF-IR, IGLF2, LOXL2, mesothelin, MS4A1, mucin SAC, MUC1, Nectin-4, neuropilin, N-glycolil GM3, PSMA, SLAMF7, TAG-72, TRAIL, TYRP1, or VEGF or any combination thereof. In some embodiments, the CAR can specifically bind to a hapten listed in TABLE 1. In some embodiments, the hapten can be selected from fluorescein, urushiol, quinone, biotin, or dinitrophenol, or a derivative thereof. In some alternatives, the first or second ligand binding domain comprises an antibody or binding fragment thereof or an scFv that is specific for 5T4, B7-H3, carbonic anhydrase IX, carcinoembryonic antigen, CA-125, CD-3, CD-19, CD-20, CD-22, CD-30, CD-33, CD-38, CD-40, CD-51, CD-52, CD-56, CD-70, CD-74, CD-79b, CD-138, CD-221, CD-319, CD-326, cell adhesion molecule 5, CTLA-4, cytokeratin polypeptides, death receptor 2, DLL4, EGFL7, EGFR, endosialin, EpCAM, FAP, FR-alpha, fibronectin, frizzled receptors, GD2, GPNMB, HER-1, HER-2, HER-3, IGF-IR, IGLF2, LOXL2, mesothelin, MS4A1, mucin SAC, MUC1, Nectin-4, neuropilin, N-glycolil GM3, PSMA, SLAMF7, TAG-72, TRAIL, TYRP1 or VEGF or any combination thereof. In some alternatives, the second ligand binding domain comprises a binding fragment of an antibody such as an antibody against a hapten listed in TABLE 1, or an antibody listed in TABLE 2. In some alternatives, the first polypeptide spacer or second polypeptide spacer or both comprises a length of 1-24, 25-50, 51-75, 76-100, 101-125, 126-150, 151-175, 176-200, 201-225, 226-250 or 251-275 amino acids. In some alternatives, the nucleic acids further comprise a leader sequence. In some alternatives, the first and/or second intracellular signaling domains comprises CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83 or CD3-zeta cytoplasmic domains or both. In some alternatives, the intracellular signaling domain comprises a portion of CD3 zeta and a portion of 4-1BB. In some alternatives, the nucleic acids further comprising a sequence encoding a marker sequence. In some alternatives, the marker is EGFRt, CD19t, or Her2tG. In some alternatives, the first and/or second transmembrane domain comprises the transmembrane domain of CD28. In some alternatives, the nucleic acids further comprise a sequence encoding a cleavable linker. In some alternatives, the linker is a ribosome skip sequence. In some alternatives, the ribosome skip sequence is P2A, T2A, E2A or F2A. In some alternatives, a plurality of vectors comprising the nucleic acids of any one of the alternatives herein are provided. In some alternatives, the chimeric antigen receptors encoded by the nucleic acids of any one of the alternatives herein or the vector of any one of the alternatives herein is provided.

### Bispecific Chimeric Antigen Receptors

In some alternatives, one or more nucleic acids for the expression of a bispecific chimeric antigen receptor is provided. In some embodiments, the nucleic acid comprises a sequence encoding a first ligand binding domain, which is specific for a tumor antigen, a Gly-Ser linker, a second ligand binding domain specific for a hapten, a polypeptide spacer, a transmembrane domain and intracellular signaling domain. In some alternatives, the first ligand binding domain is specific for 5T4, B7-H3, carbonic anhydrase IX, carcinoembryonic antigen, CA-125, CD-3, CD-19, CD-20, CD-22, CD-30, CD-33, CD-38, CD-40, CD-51, CD-52, CD-56, CD-70, CD-74, CD-79b, CD-138, CD-221, CD-319, CD-326, cell adhesion molecule 5, CTLA-4, cytokeratin polypeptides, death receptor 2, DLL4, EGFL7, EGFR, endosialin, EpCAM, FAP, FR-alpha, fibronectin, frizzled receptors, GD2, GPNMB, HER-1, HER-2, HER-3, IGF-IR, IGLF2, LOXL2, mesothelin, MS4A1, mucin SAC, MUC1, Nectin-4, neuropilin, N-glycolil GM3, PSMA, SLAMF7, TAG-72, TRAIL, TYRP1, VEGF, or other cancer expressing antigens. In some embodiments, the CAR can specifically bind to a hapten listed in TABLE 1. In some embodiments, the hapten can be selected from fluorescein, urushiol, quinone, biotin, or dinitrophenol, or a derivative thereof. In some alternatives, the first or second ligand binding domain or both comprises an antibody or binding fragment thereof or scFv. In some alternatives, the second ligand binding domain comprises a binding fragment of an antibody such as an antibody against a hapten listed in TABLE 1, or an antibody listed in TABLE 2. In some alternatives, the first polypeptide spacer or second polypeptide spacer or both comprises a length of 1-24, 25-50, 51-75, 76-100, 101-125, 126-150, 151-175, 176-200, 201-225, 226-250 or 251-275 amino acids. In some alternatives, the nucleic acid further comprises a leader sequence. In some alternatives, the intracellular signaling domain comprises CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83 or CD3-zeta cytoplasmic domains or both. In some alternatives, the intracellular signaling domain comprises a portion of CD3 zeta and a portion of 4-1BB. In some alternatives, the nucleic acid further comprises a sequence encoding a marker sequence. In some alternatives, the marker is EGFRt, CD19t, or Her2tG. In some alternatives, the transmembrane domain comprises the transmembrane domain of CD28. In some alternatives, one or more vectors for bispecific CAR expression comprising the one or more nucleic acids of any one the alternatives herein are provided. In some alternatives a bi-specific chimeric antigen receptor encoded by the nucleic acids of any one of the alternatives herein or the vector of any one of the alternatives herein is provided.

### Cells Comprising CARs or Bispecific CARs

In some alternatives, a cell comprising the one or more nucleic acids of any one of the alternatives herein, the one or more vectors of any one of the alternatives herein, or the bi-specific chimeric antigen receptor any one of the alternatives herein is provided. The nucleic acid or nucleic acids may be provided within a single vector or within a plurality of vectors in order to accommodate the payload size of two CARs. The one or more vectors may comprise any one of the alternative nucleic acids provided herein. Alternatively, the nucleic acid may be integrated using a transposon system or integrase system. The one or more nucleic acids may comprise a first sequence encoding the first chimeric antigen receptor, wherein the first chimeric antigen receptor comprises a first ligand binding domain, which is specific for a tumor antigen, a first polypeptide spacer, a first transmembrane domain and a first intracellular signaling domain; and a second sequence encoding the second chimeric antigen receptor, wherein the second chimeric antigen receptor comprises a second ligand binding domain specific for a hapten, a second polypeptide spacer, a second transmembrane domain and a second intracellular signaling domain. In some alternatives, a plurality of nucleic acids are provided, wherein the first nucleic acid comprises a first sequence encoding the first chimeric antigen receptor, wherein the first chimeric antigen receptor comprises a first ligand binding domain, which is specific for a tumor antigen, a first polypeptide spacer, a first transmembrane domain and a first intracellular signaling domain and the second nucleic acid comprises a second sequence encoding the second chimeric antigen receptor, wherein the second chimeric antigen receptor comprises a second ligand binding domain, which is specific for a hapten, a second polypeptide spacer, a second transmembrane domain and a second intracellular signaling domain, is provided. In some alternatives the first ligand binding domain is specific for a tumor cell antigen. In some alternatives, the antigen comprises 5T4, B7-H3, carbonic anhydrase IX, carcinoembryonic antigen, CA-125, CD-3, CD-19, CD-20, CD-22, CD-30, CD-33, CD-38, CD-40, CD-51, CD-52, CD-56, CD-70, CD-74, CD-79b, CD-138, CD-221, CD-319, CD-326, cell adhesion molecule 5, CTLA-4, cytokeratin polypeptides, death receptor 2, DLL4, EGFL7, EGFR, endosialin, EpCAM, FAP, FR-alpha, fibronectin, frizzled receptors, GD2, GPNMB, HER-1, HER-2, HER-3, IGF-IR, IGLF2, LOXL2, mesothelin, MS4A1, mucin SAC, MUC1, Nectin-4, neuropilin, N-glycolil GM3, PSMA, SLAMF7, TAG-72, TRAIL, TYRP1 or VEGF or any combination thereof. In some embodiments, the CAR can specifically bind to a hapten listed in TABLE 1. In some embodiments, the hapten can be selected from fluorescein, urushiol, quinone, biotin, or dinitrophenol, or a derivative thereof. In some alternatives, the first or second ligand binding domain or both comprises an antibody or binding fragment thereof or scFv. In some alternatives, the second ligand binding domain comprises a binding fragment of an antibody such as an antibody against a hapten listed in TABLE 1, or an antibody listed in TABLE 2. In some alternatives, the first polypeptide spacer or second polypeptide spacer or both comprises a length of 1-24, 25-50, 51-75, 76-100, 101-125, 126-150, 151-175, 176-200, 201-225, 226-250 or 251-275 amino acids. In some alternatives, the nucleic acid further comprises a leader sequence. In some alternatives, the first or second intracellular signaling domains or both comprises CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83 or CD3-zeta cytoplasmic domains. In some alternatives, the intracellular signaling domain comprises CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83 or CD3-zeta cytoplasmic domains. In some alternatives, the nucleic acid further comprises a sequence encoding a marker sequence. In some alternatives, the marker is EGFRt, CD19t, or Her2tG. In some alternatives, the first and/or second transmembrane domain comprises the transmembrane domain of CD28. In some alternatives, the nucleic acid further comprises a sequence encoding a cleavable linker. In some alternatives, the linker is a ribosome skip sequence. In some alternatives, the ribosome skip sequence is P2A, T2A, E2A or F2A. The cleavable linker may be in between the sequences encoding the two chimeric antigen receptors. Additionally, a cleavable linker may be used in between any one of the chimeric antigen receptors and the sequence encoding the marker protein. In some alternatives, one or more vectors for bispecific CAR expression comprising the one or more nucleic acids of any one the alternatives herein are provided. In some alternatives, a bi-specific chimeric antigen receptor encoded by the one or more nucleic acids is comprised in a cell. The one or more nucleic acids for the bi-specific chimeric antigen receptor comprises a sequence encoding a first ligand binding domain, which is specific for a tumor antigen, a Gly-Ser linker, a second ligand binding domain specific for a hapten, a polypeptide spacer, a transmembrane domain and an intracellular signaling domain. In some alternatives, the first ligand binding domain is specific for 5T4, B7-H3, carbonic anhydrase IX, carcinoembryonic antigen, CA-125, CD-3, CD-19, CD-20, CD-22, CD-30, CD-33, CD-38, CD-40, CD-51, CD-52, CD-56, CD-70, CD-74, CD-79b, CD-138, CD-221, CD-319, CD-326, cell adhesion molecule 5, CTLA-4, cytokeratin polypeptides, death receptor 2, DLL4, EGFL7, EGFR, endosialin, EpCAM, FAP, FR-alpha, fibronectin, frizzled receptors, GD2, GPNMB, HER-1, HER-2, HER-3, IGF-IR, IGLF2, LOXL2, mesothelin, MS4A1, mucin SAC, MUC1, Nectin-4, neuropilin, N-glycolil GM3, PSMA, SLAMF7, TAG-72, TRAIL, TYRP1, oro VEGF, or another antigen expressed on a cancer cell. In some embodiments, the hapten is selected from a hapten listed in TABLE 1. In some embodiments, the hapten can be selected from fluorescein, urushiol, quinone, biotin, or dinitrophenol, or a derivative thereof. In some alternatives, the first or second ligand binding domain or both comprises an antibody or binding fragment thereof or scFv. In some alternatives, the second ligand binding domain comprises a binding fragment of an antibody such as an antibody against a hapten listed in TABLE 1, or an antibody listed in TABLE 2. In some alternatives, the first polypeptide spacer or second polypeptide spacer or both comprises a length of 1-24, 25-50, 51-75, 76-100, 101-125, 126-150, 151-175, 176-200, 201-225, 226-250 or 251-275 amino acids. In some alternatives, the nucleic acid further comprises a leader sequence. In some alternatives, the intracellular signaling domain comprises CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83 or CD3-zeta cytoplasmic domains. In some alternatives, the intracellular signaling domain comprises a portion of CD3 zeta and a portion of 4-1BB. In some alternatives, the nucleic acid further comprises a sequence encoding a marker sequence. In some alternatives, the marker is EGFRt, CD19t, or Her2tG. In some alternatives, the transmembrane domain comprises the transmembrane domain of CD28. In some alternatives, the cell is a CD8+T cytotoxic lymphocyte cell selected from the group consisting of naïve CD8+ T cells, central memory CD8+ T cells, effector memory CD8+ T cells and bulk CD8+ T cells. In some alternatives, the CD8+ cytotoxic T lymphocyte cell is a central memory T cell and, wherein the central memory T cell is positive for CD45RO+, CD62L+, and CD8+. In some alternatives, the cell is a CD4+T helper lymphocyte cell selected from the group consisting of naïve CD4+ T cells, central memory CD4+ T cells, effector memory CD4+ T cells, and bulk CD4+ T cells. In some alternatives, the cell is a naïve CD4+ T cell and, wherein the naïve CD4+ T cell is positive for CD45RA+, CD62L+ and CD4+ and negative for CD45RO. In some alternatives, the cell is a precursor T cell. In some alternatives, the cell is a hematopoietic stem cell.

### Preparing Cells Comprising Two CARs or a Bi-Specific CAR

In the alternatives herein, a method of making a cell that expresses a first chimeric antigen receptor, which is specific for a hapten, and a second chimeric antigen receptor, which is specific for a tumor antigen is provided. In some instances, the method comprises introducing the one or more nucleic acids of any one of the alternatives herein or the one or more vectors of any one of the alternatives herein into a cell under conditions whereby the first and second chimeric antigen receptor are expressed. In some alternatives, a method of making a cell that expresses a bispecific chimeric antigen receptor, which is specific for a hapten and a tumor antigen is provided. The method comprises introducing the one or more nucleic acids of any one of the alternatives herein or the one or more vector of any one of the alternatives herein into a cell under conditions whereby the first and second chimeric antigen receptor are expressed is also provided. In some alternatives, the cell is a CD8+T cytotoxic lymphocyte cell selected from the group consisting of naïve CD8+ T cells, central memory CD8+ T cells, effector memory CD8+ T cells and bulk CD8+ T cells. In some alternatives, the CD8+ cytotoxic T lymphocyte cell is a central memory T cell and, wherein the central memory T cell is positive for CD45RO+, CD62L+, and CD8+. In some alternatives, the cell is a CD4+T helper lymphocyte cell selected from the group consisting of naïve CD4+ T cells, central memory CD4+ T cells, effector memory CD4+ T cells, and bulk CD4+ T cells. In some alternatives, the CD4+ helper lymphocyte cell is a naïve CD4+ T cell and, wherein the naïve CD4+ T cell is positive for CD45RA+, CD62L+ and CD4+ and negative for CD45RO. In some alternatives, the cell is a precursor T cell. In some alternatives, the cell is a hematopoietic stem cell. The one or more nucleic acids comprise a first nucleic acid comprising a first sequence encoding the first chimeric antigen receptor, wherein the first chimeric antigen receptor comprises a first ligand binding domain, which is specific for a tumor antigen, a first polypeptide spacer, a first transmembrane domain and a first intracellular signaling domain and a second nucleic acid comprising a second sequence encoding the second chimeric antigen receptor, wherein the second chimeric antigen receptor comprises a second ligand binding domain, which is specific for a hapten, a second polypeptide spacer, a second transmembrane domain and a second intracellular signaling domain. In some alternatives, the first ligand binding domain is specific for 5T4, B7-H3, carbonic anhydrase IX, carcinoembryonic antigen, CA-125, CD-3, CD-19, CD-20, CD-22, CD-30, CD-33, CD-38, CD-40, CD-51, CD-52, CD-56, CD-70, CD-74, CD-79b, CD-138, CD-221, CD-319, CD-326, cell adhesion molecule 5, CTLA-4, cytokeratin polypeptides, death receptor 2, DLL4, EGFL7, EGFR, endosialin, EpCAM, FAP, FR-alpha, fibronectin, frizzled receptors, GD2, GPNMB, HER-1, HER-2, HER-3, IGF-IR, IGLF2, LOXL2, mesothelin, MS4A1, mucin SAC, MUC1, Nectin-4, neuropilin, N-glycolil GM3, PSMA, SLAMF7, TAG-72, TRAIL, TYRP1, or VEGF or any combination thereof. In some embodiments, the hapten is selected from a a hapten listed in TABLE 1. In some embodiments, the hapten can be selected from fluorescein, urushiol, quinone, biotin, or dinitrophenol, or a derivative thereof. In some alternatives, the first or second ligand binding domain or both comprises an antibody or binding fragment thereof or scFv is specific for 5T4, B7-H3, carbonic anhydrase IX, carcinoembryonic antigen, CA-125, CD-3, CD-19, CD-20, CD-22, CD-30, CD-33, CD-38, CD-40, CD-51, CD-52, CD-56, CD-70, CD-74, CD-79b, CD-138, CD-221, CD-319, CD-326, cell adhesion molecule 5, CTLA-4, cytokeratin polypeptides, death receptor 2, DLL4, EGFL7, EGFR, endosialin, EpCAM, FAP, FR-alpha, fibronectin, frizzled receptors, GD2, GPNMB, HER-1, HER-2, HER-3, IGF-IR, IGLF2, LOXL2, mesothelin, MS4A1, mucin SAC, MUC1, Nectin-4, neuropilin, N-glycolil GM3, PSMA, SLAMF7, TAG-72, TRAIL, TYRP1 or VEGF or any combination thereof. In some alternatives, the second ligand binding domain comprises a binding fragment of an antibody such as an antibody against a hapten listed in TABLE 1, or an antibody listed in TABLE 2. In some alternatives, the first polypeptide spacer or second polypeptide spacer or both comprises a length of 1-24, 25-50, 51-75, 76-100, 101-125, 126-150, 151-175, 176-200, 201-225, 226-250 or 251-275 amino acids. In some alternatives, the nucleic acids further comprise a leader sequence. In some alternatives, the first and/or second intracellular signaling domains comprises CD27, CD28, 4-1BB, OX40, CD30, CD40, ICOS, lymphocyte function-associated antigen-1 (LFA-1), CD2, CD7, LIGHT, NKG2C, B7-H3, or a ligand that specifically binds with CD83 or CD3-zeta cytoplasmic domains. In some alternatives, the intracellular signaling domain comprises a portion of CD3 zeta and a portion of 4-1BB. In some alternatives, the nucleic acids further comprising a sequence encoding a marker sequence. In some alternatives, the marker is EGFRt, CD19t, or Her2tG. In some alternatives, the first and/or second transmembrane domain comprises the transmembrane domain of CD28. In some alternatives, the nucleic acids further comprise a sequence encoding a cleavable linker. In some alternatives, the linker is a ribosome skip sequence. In some alternatives, the ribosome skip sequence is P2A, T2A, E2A or F2A. The nucleic acid for the bispecific chimeric antigen receptor comprises a sequence encoding a first ligand binding domain, which is specific for a tumor antigen, a Gly-Ser linker, a second ligand binding domain specific for a hapten, a polypeptide spacer, a transmembrane domain and intracellular signaling domain. In some alternatives, a plurality of vectors comprising the one or more nucleic acids of any one of the alternatives herein are provided.

T lymphocytes can be collected in accordance with known techniques and enriched or depleted by known techniques such as by affinity binding to antibodies, flow cytometry and/or immunomagnetic selection. After enrichment and/or depletion steps, in vitro expansion of the desired T lymphocytes can be carried out in accordance with known techniques or variations thereof that will be apparent to those skilled in the art. In some alternatives, the T cells are autologous T cells obtained from the patient.

For example, the desired T cell population or subpopulation can be expanded by adding an initial T lymphocyte population to a culture medium in vitro, and then adding to the culture medium feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture (e.g. for a time sufficient to expand the numbers of T cells). The non-dividing feeder cells can comprise gamma-irradiated PBMC feeder cells. In some alternatives, the PBMC are irradiated with gamma rays in the range of 3000 to 3600 rads to prevent cell division. In some alternatives, the PBMC are irradiated with gamma rays of 3000, 3100, 3200, 3300, 3400, 3500 or 3600 rads or any value of rads between any two endpoints of any of the listed values to prevent cell division. The order of addition of the T cells and feeder cells to the culture media can be reversed if desired. The culture can typically be incubated under conditions of temperature and the like that are suitable for the growth of T lymphocytes. For the growth of human T lymphocytes, for example, the temperature will generally be at least 25 degrees Celsius, preferably at least 30 degrees, more preferably 37 degrees. In some alternatives, the temperature for the growth of human T lymphocytes is 22, 24, 26, 28, 30, 32, 34, 36, 37 degrees Celsius or any other temperature between any two endpoints of any of the listed values.

The T lymphocytes expanded may include CD8+ cytotoxic T lymphocytes (CTL) and CD4+ helper T lymphocytes that can be specific for an antigen present on a human tumor or a pathogen. In some alternatives, the cells include precursor T cells. In some alternatives, the cells are hematopoietic stem cells.

In some alternatives, the expansion method can further comprise adding non-dividing EBV-transformed lymphoblastoid cells (LCL) as feeder cells. LCL can be irradiated with gamma rays in the range of 6000 to 10,000 rads. In some alternatives, the LCL are irradiated with gamma rays in of 6000, 6500, 7000, 7500, 8000, 8500, 9000, 9500 or 10,000 rads or any amount of rads between two endpoints of any of the listed values. The LCL feeder cells can be provided in any suitable amount, such as a ratio of LCL feeder cells to initial T lymphocytes of at least 10:1.

In some alternatives, the expansion method can further comprise adding antiCD3 and/or anti CD28 antibody to the culture medium (e.g., at a concentration of at least 0.5 ng/ml). In some alternatives, the expansion method can further comprise adding IL-2 and/or IL-15 to the culture medium (e.g., wherein the concentration of IL-2 is at least 10 units/ml). After isolation of T lymphocytes both cytotoxic and helper T lymphocytes can be sorted into naïve, memory, and effector T cell subpopulations either before or after expansion.

CD8+ cells can also be obtained by using standard methods. In some alternatives, CD8+ cells are further sorted into naïve, central memory, and effector memory cells by identifying cell surface antigens that are associated with each of those types of CD8+ cells. In some alternatives, memory T cells are present in both CD62L+ and CD62L− subsets of CD8+ peripheral blood lymphocytes. PBMC are sorted into CD62L− CD8+ and CD62L+CD8+ fractions after staining with antiCD8 and antiCD62L antibodies. In some alternatives, the expression of phenotypic markers of central memory T_{CM} include CD45RO, CD62L, CCR7, CD28, CD3, and/or CD127 and are negative or low for granzyme B. In some alternatives, central memory T cells are CD45RO+, CD62L+, and/or CD8+ T cells. In some alternatives, effector TE are negative for CD62L, CCR7, CD28, and/or CD127, and positive for granzyme B and/or perforin. In some alternatives, naïve CD8+T lymphocytes are characterized by the expression of phenotypic markers of naïve T cells including CD62L, CCR7, CD28, CD3, CD127, and/or CD45RA.

CD4+T helper cells are sorted into naïve, central memory, and effector cells by identifying cell populations that have cell surface antigens. CD4+ lymphocytes can be obtained by standard methods. In some alternatives, naïve CD4+T lymphocytes are CD45RO−, CD45RA+, CD62L+, and/or CD4+ T cells. In some alternatives, central memory CD4+ cells are CD62L+ and/or CD45RO+. In some alternatives, effector CD4+ cells are CD62L− and/or CD45RO−.

Whether a cell or cell population is positive for a particular cell surface marker can be determined by flow cytometry using staining with a specific antibody for the surface marker and an isotype matched control antibody. A cell population negative for a marker refers to the absence of significant staining of the cell population with the specific antibody above the isotype control, positive refers to uniform staining of the cell population above the isotype control.

In some alternatives, a decrease in expression of one or markers refers to loss of 1 log 10 in the mean fluorescence intensity and/or decrease of percentage of cells that exhibit the marker of at least 20% of the cells, 25% of-the cells, 30% of the cells, 35% of the cells, 40% of the cells, 45% of the cells, 50% of the cells, 55% of the cells, 60% of the cells, 65% of the cells, 70% of the cells, 75% of the cells, 80% of the cells, 85% of the cells, 90% of the cell, 95% of the cells, and 100% of the cells or any % between 20 and 100% when compared to a reference cell population. In some alternatives, a cell population positive for one or markers refers to a percentage of cells that exhibit the marker of at least 50% of the cells, 55% of the cells, 60% of the cells, 65% of the cells, 70% of the cells, 75% of the cells, 80% of the cells, 85% of the cells, 90% of the cell, 95% of the cells, or 100% of the cells or any % between 50 and 100% when compared to a reference cell population.

In some alternatives, populations of CD4+ and CD8+ that are antigen specific can be obtained by stimulating naïve or antigen specific T lymphocytes with antigen. For example, antigen-specific T cell lines or clones can be generated to Cytomegalovirus antigens by isolating T cells from infected subjects and stimulating the cells in vitro with the same antigen. Naïve T cells can also be used. Any number of antigens from tumor cells can be utilized as targets to elicit T cell responses. In some alternatives, the adoptive cellular immunotherapy compositions are useful in the treatment of a disease or disorder including a solid tumor and/or hematologic malignancy.

Additional methods for stimulating cells ex vivo are also contemplated. Cells that comprise a hapten or a hapten conjugated to a bead may also be used to stimulate the cells prior to use as a method of treatment. The CAR T bearing cells may be stimulated using a hapten bearing cell that is made by standard known techniques of by exposure to a hapten conjugated support (e.g. on a bead, well or dish).

### Stimulation of the Chimeric Antigen Receptor In Vivo

A method of stimulating or re-stimulating chimeric antigen receptor (CAR)-bearing T-cells in a subject suffering from a disease, such as cancer is also provided. The method comprises providing the cell of any one of the alternative cells provided herein, to the subject, monitoring the subject for inhibition of said disease; and providing hapten antigen presenting cells (H-APC) to the subject, wherein said subject is optionally, selected to receive a CAR T cell therapy utilizing CAR T cells having receptors specific for an antigen associated with the disease, such as a tumor antigen. The cell may comprise the one or more vectors or the one or more nucleic acids of any one of the alternatives herein, or the bi-specific chimeric antigen receptor any one of the alternatives herein. The one or more nucleic acid or nucleic acids may be provided within a single vector or within a plurality of vectors in order to accommodate the payload size of two CARs. The one or more vectors may comprise any one of the alternative nucleic acids provided herein. Alternatively, the nucleic acid may be integrated using a transposon system or integrase system. The nucleic acid may comprise a first sequence encoding the first chimeric antigen receptor, wherein the first chimeric antigen receptor comprises a first ligand binding domain, which is specific for a tumor antigen, a first polypeptide spacer, a first transmembrane domain and a first intracellular signaling domain; and a second sequence encoding the second chimeric antigen receptor, wherein the second chimeric antigen receptor comprises a second ligand binding domain specific for a hapten, a second polypeptide spacer, a second transmembrane domain and a second intracellular signaling domain. In some alternatives, a plurality of nucleic acids are provided, wherein the first nucleic acid comprises a first sequence encoding the first chimeric antigen receptor, wherein the first chimeric antigen receptor comprises a first ligand binding domain, which is specific for a tumor antigen, a first polypeptide spacer, a first transmembrane domain and a first intracellular signaling domain and the second nucleic acid comprises a second sequence encoding the second chimeric antigen receptor, wherein the second chimeric antigen receptor comprises a second ligand binding domain, which is specific for a hapten, a second polypeptide spacer, a second transmembrane domain and a second intracellular signaling domain, is provided. In some alternatives the first ligand binding domain is specific for a tumor cell antigen. In some alternatives a bi-specific chimeric antigen receptor encoded by the nucleic acids is comprised in a cell. The nucleic acid for the bi-specific chimeric antigen receptor comprises a sequence encoding a first ligand binding domain, which is specific for a tumor antigen, a Gly-Ser linker, a second ligand binding domain specific for a hapten, a polypeptide spacer, a transmembrane domain and intracellular signaling domain. In some alternatives, the H-APC is generated from healthy cells of the subject by ex vivo labeling the healthy cells with a hapten.

The H-APC is created from healthy cells of a patient, such as a human, or cells that are compatible with said patient, and ex vivo labeling of the cells with a hapten. Examples of haptens are fluorescein, urushiol, quinone, or biotin. There are many ways to label a cell with a hapten, e.g. chemical, peptide, aptamer, lipid, or protein. An example of how to load cells with a hapten comprises incubation of a fluorescein-lipid overnight with cells of interest. One benefit to the use of fluorescein as a hapten is its fluorescence. Therefore, hapten integration can be monitored by the fluorescence of the fluorescein moiety via flow cytometry. Thus, after incubation excess fluorescein-lipid can be removed, a fraction of the cells can be subjected to flow analysis to analyze hapten integration, and the remaining cells can be used for patient infusion. Post patient infusion H-APCs will slowly lose the hapten (metabolized, defused from the surface, etc.) and return to their original healthy cell form if not targeted by a CAR T cell, demonstrating a layer of safety in this approach. The hapten may be bound to a lipid for integration into the cell to make an H-APC.

In some embodiments, the hapten is selected from a hapten listed in TABLE 1. In some embodiments, the hapten can be selected from fluorescein, urushiol, quinone, biotin, or dinitrophenol, or a derivative thereof. In some alternatives, the monitoring and the providing steps are repeated. In some alternatives, the subject has a cancer. In some alternatives, the cancer is solid tumor. In some alternatives, the subject is selected or identified to receive a cancer therapy e.g., by conventional clinical evaluation and diagnostic testing or both. In some alternatives, the subject is subjected to combination therapy, such as chemotherapy or radiation.

### Ex Vivo Stimulation of Cells

In some alternatives, a method of stimulating or re-stimulating chimeric antigen receptor (CAR)-bearing T-cells ex vivo is provided. In some instances, the method comprises providing the cell of any one of the alternatives herein, providing hapten antigen presenting cells (H-APC) or a hapten, mixing the cell and the H-APC cells, thereby making activated cells and isolating the activated cells. The cell may comprise the one or more vectors or nucleic acids of any one of the alternatives herein, or the bi-specific chimeric antigen receptor any one of the alternatives herein. The one or more nucleic acid or nucleic acids may be provided within a single vector or within a plurality of vectors in order to accommodate the payload size of two CARs. The one or more vectors may comprise any one of the alternative nucleic acids provided herein. Alternatively, the nucleic acid may be integrated using a transposon system or integrase system. The one or more nucleic acids may comprise a first sequence encoding the first chimeric antigen receptor, wherein the first chimeric antigen receptor comprises a first ligand binding domain, which is specific for a tumor antigen, a first polypeptide spacer, a first transmembrane domain and a first intracellular signaling domain; and a second sequence encoding the second chimeric antigen receptor, wherein the second chimeric antigen receptor comprises a second ligand binding domain specific for a hapten, a second polypeptide spacer, a second transmembrane domain and a second intracellular signaling domain. In some alternatives, a plurality of nucleic acids are provided, wherein the first nucleic acid comprises a first sequence encoding the first chimeric antigen receptor, wherein the first chimeric antigen receptor comprises a first ligand binding domain, which is specific for a tumor antigen, a first polypeptide spacer, a first transmembrane domain and a first intracellular signaling domain and the second nucleic acid comprises a second sequence encoding the second chimeric antigen receptor, wherein the second chimeric antigen receptor comprises a second ligand binding domain, which is specific for a hapten, a second polypeptide spacer, a second transmembrane domain and a second intracellular signaling domain, is provided. In some alternatives the first ligand binding domain is specific for a tumor cell antigen. In some alternatives a bi-specific chimeric antigen receptor encoded by the nucleic acids is comprised in a cell. The nucleic acid for the bi-specific chimeric antigen receptor comprises a sequence encoding a first ligand binding domain, which is specific for a tumor antigen, a Gly-Ser linker, a second ligand binding domain specific for a hapten, a polypeptide spacer, a transmembrane domain and intracellular signaling domain. In some embodiments, the hapten is selected from a hapten listed in TABLE 1. In some alternatives, the H-APC comprises a hapten, wherein the hapten is selected from a hapten listed in TABLE 1. In some embodiments, the hapten can be selected from fluorescein, urushiol, quinone, biotin, or dinitrophenol, or a derivative thereof.

In some alternatives, isolating the activated cells comprises affinity isolation with hapten complexed affinity beads. In some alternatives, isolating the activated cells comprises affinity isolation with EGFRt, CD19t, or Her2tG complexed affinity beads.

In some embodiments, a CAR can have the structure: antiFL(FITC-E2)scFv-IgG4hinge-CH2(L235D, N297Q)-CH3--CD28tm/41BB-zeta-T2A-EGFRt. Example amino acid sequences that can be used with embodiments of the methods and compositions provided herein are listed in TABLE 4.

TABLE 4

| SEQ ID NO: | Sequence |
|---|---|
| SEQ ID NO: 11 | MLLLVTSLLLCELPHPAFLLIP |
| GM-CSF | |
| scFv | See TABLE 3 for examples |
| SEQ ID NO: 12 | ESKYGPPCPPCPAPEFDGGPSVFLFPPKPKDTLMISRTPEVTCVVVDVSQEDPEVQF |
| Spacer (long): | NWYVDGVEVHNAKTKPREEQFNSTYRVVSVLTVLHQDWLNGKEYKCKVSNKGLPS |
| IgG4hinge- | SIEKTISKAKGQPREPQVYTLPPSQEEMTKNQVSLTCLVKGFYPSDIAVEWESNGQPE |
| CH2(L235D)-CH3 | NNYKTTPPVLDSDGSFFLYSRLTVDKSRWQEGNVFSCSVMHEALHNHYTQKSLSLSL |
| | GK |
| SEQ ID NO: 13 | ESKYGPPCPPCPGQPREPQVYTLPPSQEEMTKNQVSLTCLVKGFYPSDIAVEWESNG |
| Spacer (medium): | QPENNYKTTPPVLDSDGSFFLYSRLTVDKSRWQEGNVFSCSVMHEALHNHYTQKSL |
| IgG4 hinge-CH3 | SLSLGK |
| SEQ ID NO: 14 | ESKYGPPCPPCP |
| Spacer (short): IgG4 | |
| hinged | |
| SEQ ID NO: 15 | MFWVLVVVGGVLACYSLLVTVAFIIFWV |
| CD28tm | |
| SEQ ID NO: 16 | KRGRKKLLYIFKQPFMRPVQTTQEEDGCSCRFPEEEEGGCEL |
| 4-1BB | |
| SEQ ID NO: 17 | RVKFSRSADAPAYQQGQNQLYNELNLGRREEYDVLDKRRGRDPEMGGKPRRKNP |
| CD3 zeta | QEGLYNELQKDKMAEAYSEIGMKGERRRGKGHDGLYQGLSTATKDTYDALHMQAL |
| | PPR |
| SEQ ID NO: 18 | GGGEGRGSLLTCGDVEENPGP |
| T2A | |
| SEQ ID NO: 19 | MLLLVTSLLLCELPHPAFLLIPRKVCNGIGIGEFKDSLSINATNIKHFKNCTSISGDLHILP |
| GM-CSF receptor ss | VAFRGDSFTHTPPLDPQELDILKTVKEITGFLLIQAWPENRTDLHAFENLEIIRGRTKQ |
| to EGFRt | HGQFSLAVVSLNITSLGLRSLKEISDGDVIISGNKNLCYANTINWKKLFGTSGQKTKIIS |
| | NRGENSCKATGQVCHALCSPEGCWGPEPRDCVSCRNVSRGRECVDKCNLLEGEPR |
| | EFVENSECIQCHPECLPQAMNITCTGRGPDNCIQCAHYIDGPHCVKTCPAGVMGEN |
| | NTLVWKYADAGHVCHLCHPNCTYGCTGPGLEGCPTNGPKIPSIATGMVGALLLLLV |
| | VALGIGLFM |
| SEQ ID NO: 20 | MVGSLNCIVAVSQNMGIGKNGDFPWPPLRNESRYFQRMTTTSSVEGKQNLVIMG |
| DHFRdm | KKTWFSIPEKNRPLKGRINLVLSRELKEPPQGAHFLSRSLDDALKLTEQPELANKVDM |
| | VWIVGGSSVYKEAMNHPGHLKLFVTRIMQDFESDTFFPEIDLEKYKLLPEYPGVLSDV |
| | QEEKGIKYKFEVYEKND |
| SEQ ID NO: 21 | MLLLVTSLLLCELPHPAFLLIPDVQLQESGPSLVKPSQSLSLTCTVTGYSITSDFAWNW |
| Dual CAR sequence: | IRQFPGNKLEWMGYISYSGNTRYNPSLKSRISITRDTSKNQFFLQLNSVTIEDTATYYC |
| mAb806 VHVLscFv- | VTAGRGFPYWGQGTLVTVSAGSTSGSGKPGSGEGSTKGDILMTQSPSSMSVSLGD |
| IgG4hinge- | TVSITCHSSQDINSNIGWLQQRPGKSFKGLIYHGTNLDDEVPSRFSGSGSGADYSLTIS |
| CD28tm/CD28gg- | SLESEDFADYYCVQYAQFPWTFGGGTKLEIKRESKYGPPCPPCPMFWVLVVVGGVL |
| Zeta-T2A-EGFRt-P2- | ACYSLLVTVAFIIFWVRSKRSRGGHSDYMNMTPRRPGPTRKHYQPYAPPRDFAAYR |
| AntiFL(FITC-E2 | SRVKFSRSADAPAYQQGQNQLYNELNLGRREEYDVLDKRRGRDPEMGGKPRRKNP |
| Tyr100gAla)scFv- | QEGLYNELQKDKMAEAYSEIGMKGERRRGKGHDGLYQGLSTATKDTYDALHMQAL |
| IgG4hinge- | PPRLEGSGEGRGSLLTCGDVEENPGPMLLLVTSLLLCELPHPAFLLIPRKVCNGIGIGEF |
| CH2(L235D, | KDSLSINATNIKHFKNCTSISGDLHILPVAFRGDSFTHTPPLDPQELDILKTVKEITGFLLI |
| N297Q)-CH3-- | QAWPENRTDLHAFENLEIIRGRTKQHGQFSLAVVSLNITSLGLRSLKEISDGDVIISGN |
| CD28tm/41BB-zeta- | KNLCYANTINWKKLFGTSGQKTKIISNRGENSCKATGQVCHALCSPEGCWGPEPRD |
| T2A-DHFRdm- | CVSCRNVSRGRECVDKCNLLEGEPREFVENSECIQCHPECLPQAMNITCTGRGPDN |
| epHIV7.2 | CIQCAHYIDGPHCVKTCPAGVMGENNTLVWKYADAGHVCHLCHPNCTYGCTGPG |
| | LEGCPTNGPKIPSIATGMVGALLLLLVVALGIGLFMGSGATNFSLLKQAGDVEENPG |
| | PMLLLVTSLLLCELPHPAFLLIPSVLTQPSSVSAAPGQKVTISCSGSTSNIGNNYVSWY |
| | QQHPGKAPKLMIYDVSKRPSGVPDRFSGSKSGNSASLDISGLQSEDEADYYCAAWD |
| | DSLSEFLFGTGTKLTVLGGGGGSGGGGSGGGGSQVQLVESGGNLVQPGGSLRLSCA |
| | ASGFTFGSFSMSWVRQAPGGGLEWVAGLSARSSLTHYADSVKGRFTISRDNAKNS |
| | VYLQMNSLRVEDTAVYYCARRSYDSSGYWGHFASYMDVWGQGTLVTVSESKYGP |
| | PCPPCPAPEFDGGPSVFLFPPKPKDTLMISRTPEVTCVVVDVSQEDPEVQFNWYVD |
| | GVEVHNAKTKPREEQFQSTYRVVSVLTVLHQDWLNGKEYKCKVSNKGLPSSIEKTIS |
| | KAKGQPREPQVYTLPPSQEEMTKNQVSLTCLVKGFYPSDIAVEWESNGQPENNYKT |
| | TPPVLDSDGSFFLYSRLTVDKSRWQEGNVFSCSVMHEALHNHYTOKSLSLSLGKMF |
| | WVLVVVGGVLACYSLLVTVAFIIFWVKRGRKKLLYIFKQPFMRPVQTTQEEDGCSCR |
| | FPEEEEGGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLSTATKDT |
| | YDALHMQALPPRGSGEGRGSLLTCGDVEENPGPMVGSLNCIVAVSQNMGIGKNG |
| | DFPWPPLRNESRYFQRMTTTSSVEGKQNLVIMGKKTWFSIPEKNRPLKGRINLVLSR |
| | ELKEPPQGAHFLSRSLDDALKLTEQPELANKVDMVWIVGGSSVYKEAMNHPGHLKL |
| | FVTRIMQDFESDTFFPEIDLEKYKLLPEYPGVLSDVQEEKGIKYKFEVYEKND |

### EXAMPLES

### Example 1—Preparation of Cells Having Tethered Haptens

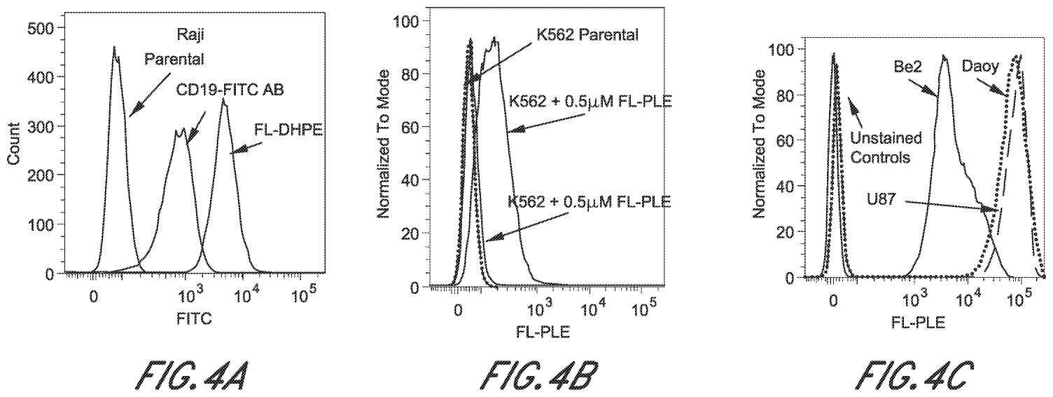

Hapten-labeled cells were prepared by either attaching the hapten, fluorescein (FL), to the cells via an integrated phospholipid or via an antibody. CD19+ Raji cells (lymphoma cell line) were incubated either overnight with 5 μM FL-DHPE (FIG. 3B), or for 20 min with an antiCD19 antibody labeled with fluorescein isothiocyanate (FITC). The cells were washed, stained, and analyzed by flow cytometry for the presence of FL. Both cells showed a positive shift for the hapten, FL, compared to untreated control cells. The levels of FL were higher for cells treated with FL-DHPE compared to cells treated with the antiCD19 antibody (FIG. 4A). This was consistent with different tethering techniques providing different levels of hapten on the surface of a cell.

K562 cells (leukemia cell line) were incubated overnight with either 0.5 μM or 5 μM FL-PLE (FIG. 3A) in the presence of FBS which can reduce the amount of integration of a phospholipid into a cell surface. The level of integration of FL-PLE into the cells was analyzed by flow cytometry. Higher levels of FL were detected in cells treated with 5 μM FL-PLE compared with cells treated with 0.5 μM FL-PLE (FIG. 4B). Higher levels of FL were detected in cells treated with 0.5 μM FL-PLE compared with untreated control cells. Thus, the concentration of FL-PLE can be modulated to change the level of FL on a cell surface. By changing the concentration of a tethering agent, such as FL-PLE, the density of a hapten, such as FL, on the surface of the cell can also change.

Be2 cells (neuroblastoma cell line), U87 cells (glioblastoma cell line), and daoy cells (medulloblastoma cell line) were incubated overnight with 5 μM FL-PLE and analyzed by flow cytometry. FL-PLE integrated into each cell line, with U87 cells and doay cells having higher levels of FL than Be2 cells (FIG. 4C). This demonstrated that different cell types incorporate FL-PLE and can incorporate FL-PLE at different levels.

### Example 2—Accessibility of Tethered Haptens

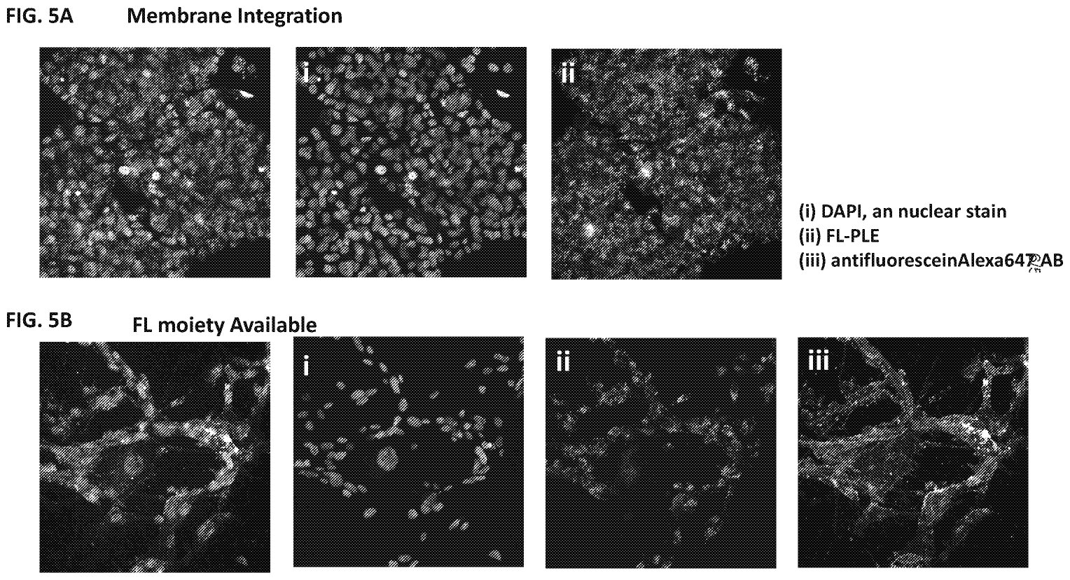

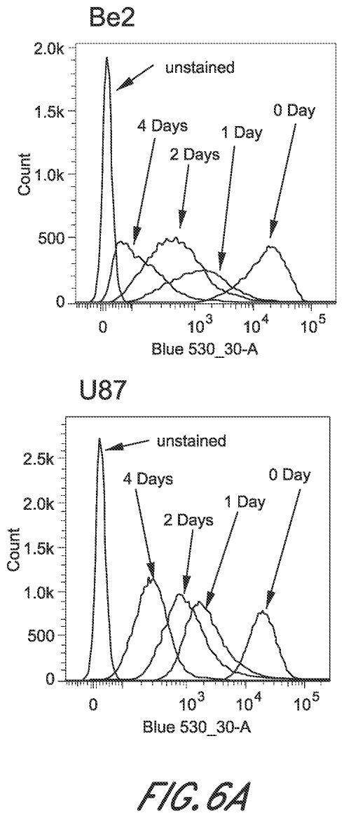

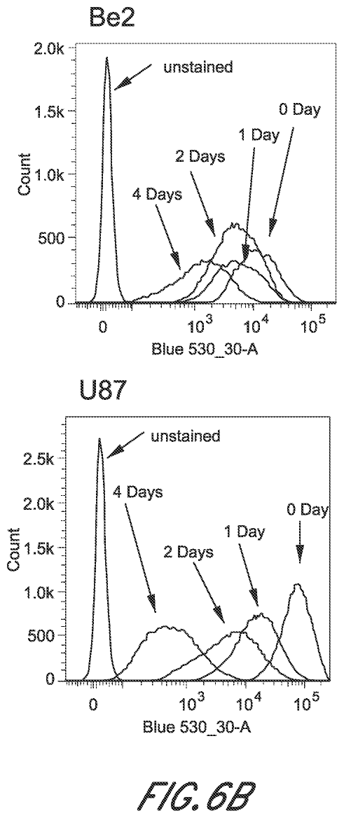

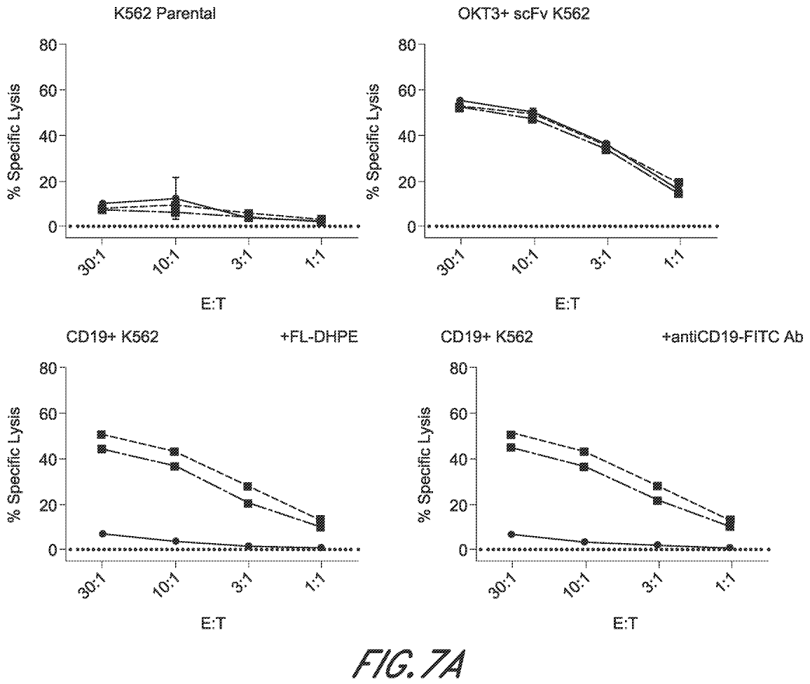

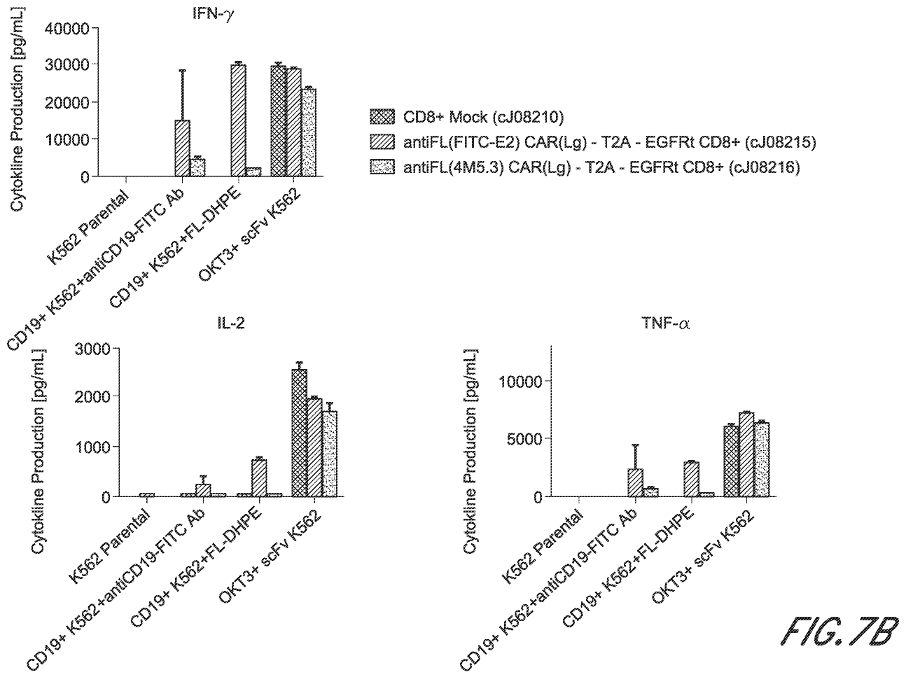

In order to confirm extracellular accessibility of loaded hapten on a cell, U87 cells were incubated overnight with 5 μM FL-PLE, then imaged by confocal microscopy to confirm the location of the FL moiety in the cells. Cell nuclei were stained with DAPI. Green fluorescent staining was observed throughout the surfaces of cells. Thus, the FL-PLE integrated over the whole cell surface (FIG. 5A). Shown on the left is a full overlay confocal image. To the right of each full overlay confocal image is a grey scale image for each layer ((i) nucleus and (ii) FL-PLE) making up the full overlay confocal image.

To determine the accessibility of the FL moiety on the cell surface, cells labeled with FL-PLE were stained with an antifluorescein antibody conjugated with an Alexa Fluor 647 fluorophore. Anti-fluorecein antibody staining was observed throughout the surfaces of cells (FIG. 5B). This confirmed that the FL moiety was accessible for extracellular binding. Shown on the left is a full overlay confocal image. To the right of each full overlay confocal image is a grey scale image for each layer ((i) nucleus, (ii) FL-PLE, and (iii) antifluorescein-Alexa Fluor 647 antibody) making up the full overlay confocal image.

### Example 3—Cell Surface Retention of Tethered Haptens

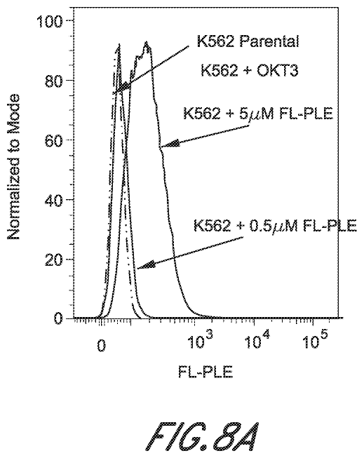

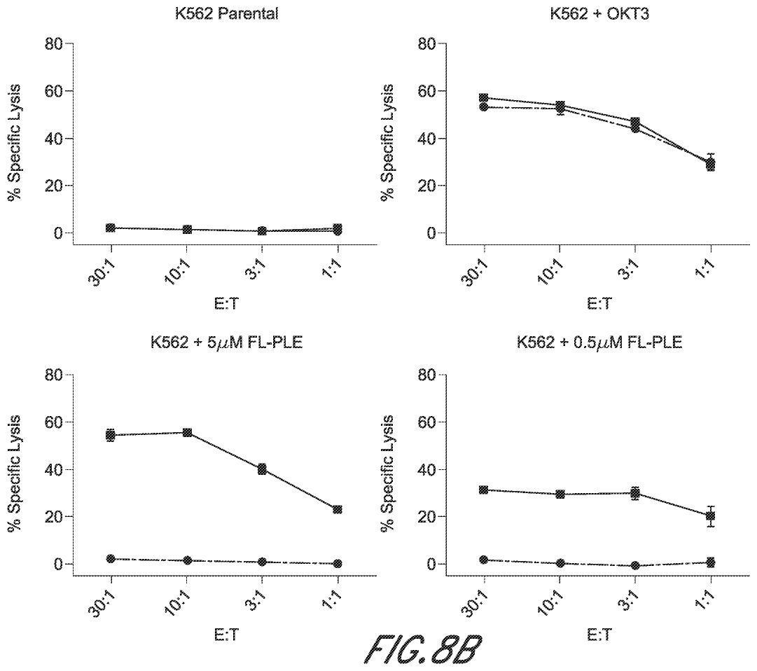

Be2 cells or U87 cells were incubated overnight in the presence of either 5 μM FL-DHPE or 5 μM FL-PLE. Cells were washed to remove any residual FL-DHPE or FL-PLE, and then cultured in fresh media for up to 4 days. Cells were analyzed by flow cytometry. Cells treated with either FL-DHPE or FL-PLE retained FL over a period of at least 4 days (FIG. 6A and FIG. 6B). Cells treated with FL-PLE had higher levels of FL at 4 days compared to cells treated with FL-DHPE. This was consistent with different tethering agents, such as phospholipids, providing different periods for a hapten to be present on the surface of a cell.

### Example 4—Recognition of Tethered Haptens, and Activation of Antihapten CAR T Cells

Hapten-labeled cells were prepared. CD19+K562 cells were incubated either overnight with 5 μM FL-DHPE or for 20 min with an CD19 antibody labeled with FITC. The hapten-labeled cells were incubated with either one of two antiFL CAR T cells (FITC-E2 scFv, or 4M5.3 scFv). Cytotoxicity, cytokine release, and proliferation assays were performed with the CAR T cells with methods substantially similar to that described in Hudecek M, et al., (2013). Hudecek M, et al., (2013) Clin Cancer Res. 19:3153-64, which is incorporated by reference in its entirety.

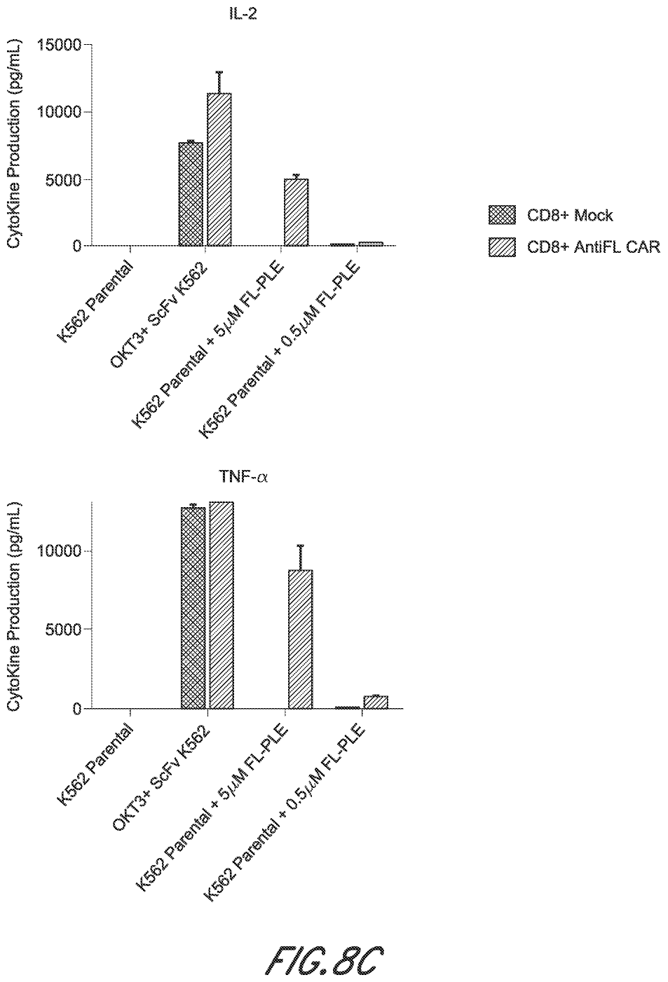

A chromium release assay was used to determine the lytic capabilities of the antiFL CAR T cells against the hapten-labelled cells. Unlabeled control K562 cells did not induce lysis with the antiFL(FITC-E2) CAR T cells, or antiFL(4M5.3) CAR T cells (FIG. 7A, top left panel). A positive control which included the use of OKT3 cells which can activate T cells through the TCR demonstrated that lysis could be induced with the antiFL(FITC-E2) CAR T cells, or antiFL(4M5.3) CAR T (FIG. 7A, top right panel). Both hapten-labeled cells induced lysis by each one of the two antiFL CAR T cells (FIG. 7A, lower panels).

The levels of cytokines released by the antiFL CAR T cells were determined. Both hapten-labeled cells induced the release of IFN-γ, IL-2 and TNF-α in contact with the antiFL(FITC-E2) CAR T cell (FIG. 7B). The levels of released IFN-γ, and TNF-α were lower for hapten-labeled cells contacted with the antiFL(4M5.3) CAR T cell. Also, there was a trend for hapten-labeled cells prepared with FL-DHPE inducing higher levels of cytokine release compared to hapten-labeled cells prepared with an CD19 antibody labeled with FITC.

### Example 5—Recognition of Tethered Haptens, and Activation of Antihapten CAR T Cells

Hapten-labeled cells were prepared. K562 cells were incubated overnight with either with either 0.5 μM or 5 μM FL-PLE. Cell integration of FL-PLE was analyzed by flow cytometry. Hapten-labeled cells were incubated with antiFL CAR T cells, and the ability of the hapten-labeled cells to induce specific lysis and cytokine release activities of the antiFL CAR T cells were measured.

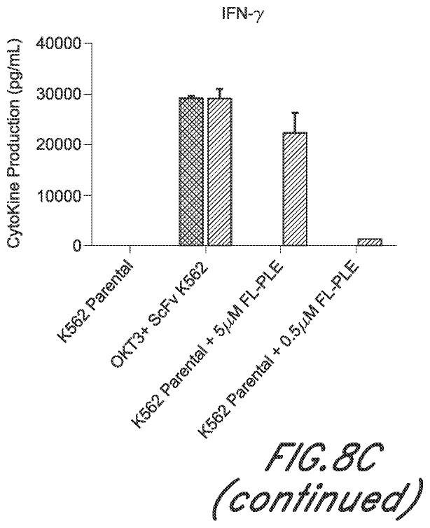

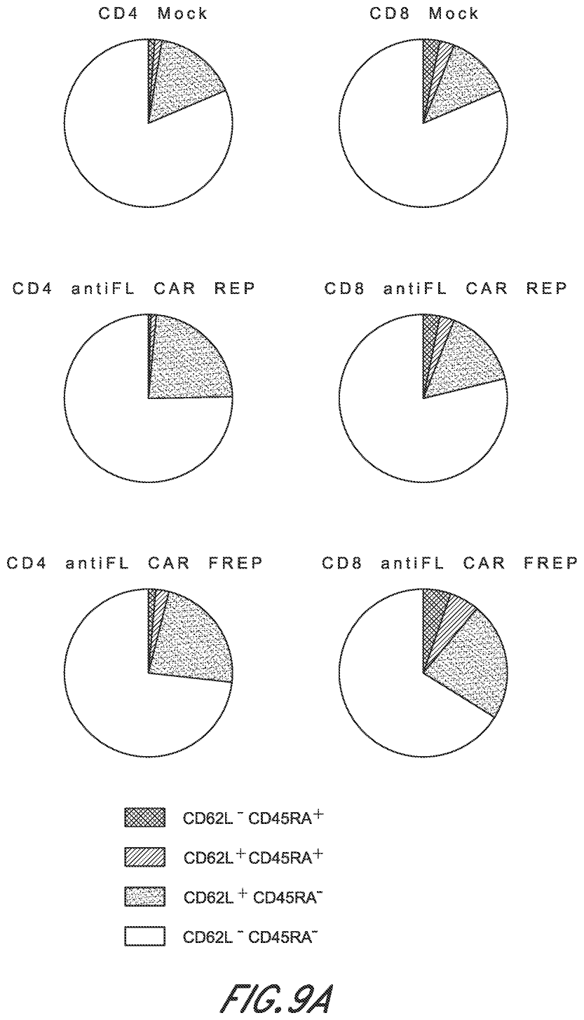

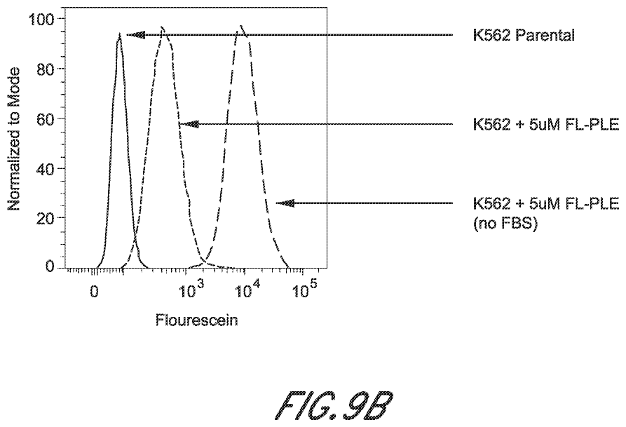

Higher levels of FL were detected in cells treated with 5 μM FL-PLE compared with cells treated with 0.5 μM FL-PLE, or untreated control cell (FIG. 8A). Higher levels of lysis and cytokine release were also observed for cells treated with 5 μM FL-PLE compared with cells treated with 0.5 μM FL-PLE, or untreated control cell (FIG. 8B and FIG. 8C). Thus, FL-PLE treated cells having a tethered extracellular FL moiety are recognized by antiFL CAR T cells and can activate the antiFL CAR T cells. The levels of antiFL CAR T cells activation may be associated with the levels of FL on the surface of the hapten-labeled cell.

### Example 6—In Vitro Expansion of Antihapten CAR T Cells

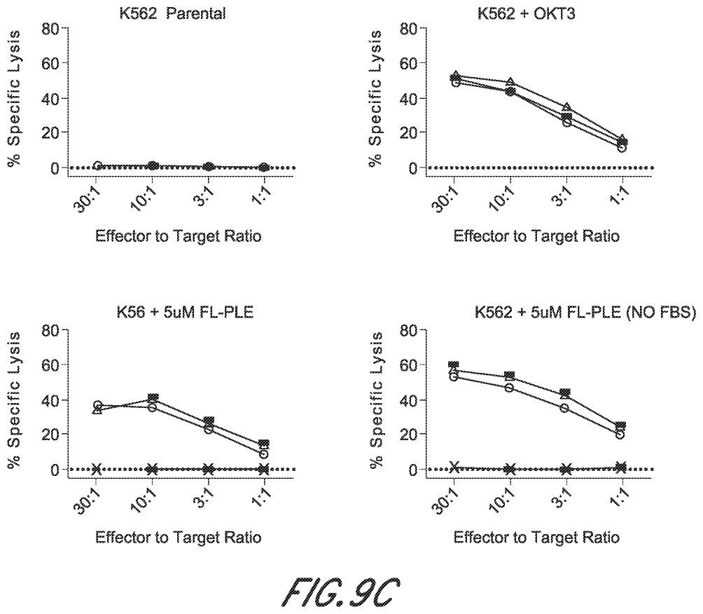

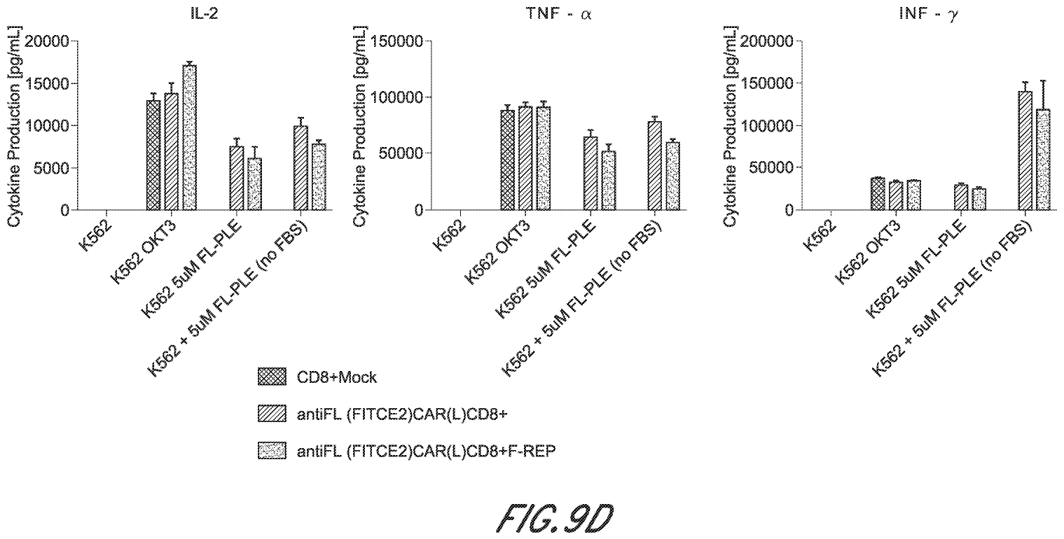

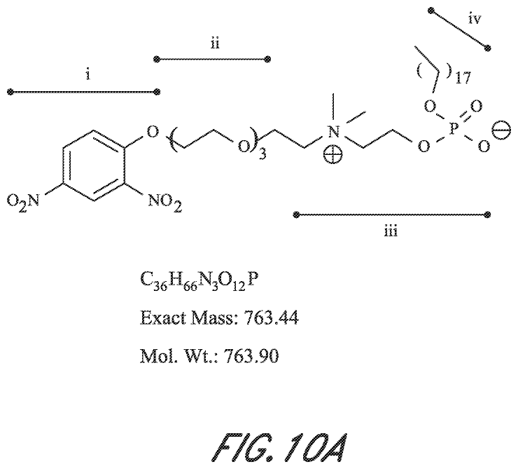

CD4+ and CD8+ antiFL CAR T cells were generated by transducing vectors into T cells. After 18 days, the transduced cells were expanded for a first time using a standard rapid expansion protocol (REP) using irradiated TM-LCL and PBMCs. The expanded cells were expanded a second time by either using a standard REP, or a fluorescein REP (FREP). For the FREP, the cells were incubated with feeder cells that had been treated with FL-PLE. After 14 days of second expansion, cells were analyzed by flow cytometry, specific lysis assays, and cytokine release assays. For the specific lysis assays and cytokine release assays, the expanded antiFL CAR T cells were incubated with K562 cells that had been incubated with FL-PLE overnight. Cell integration of FL-PLE was analyzed by flow cytometry (FIG. 9B)

Cells expanded with either REP or FREP expressed similar phenotypic markers (FIG. 9A). CD8+ antiFL CAR T cells that had been expanded using FREP had substantially similar cytotoxic activities to CD8+ antiFL CAR T cells that had been expanded using REP (FIG. 9C). CD8+ antiFL CAR T cells that had been expanded using FREP also had substantially similar cytokine release activities to CD8+ antiFL CAR T cells that had been expanded using REP (FIG. 9D). CD4+ antiFL CAR T cells that had been expanded using FREP also had substantially similar cytotoxic activities and cytokine release activities to CD4+ antiFL CAR T cells that had been expanded using REP. Therefore, cells labeled with a hapten, such as FL, can induce expansion of CAR T cells, and such expanded CAR T cells have substantially similar activity to CAR T cells expanded using irradiated TM-LCL and PBMCs.

### Example 7—Generation of Cells with Tethered Extracellular Exposed Haptens Specifically DNP Using DNP-PLE

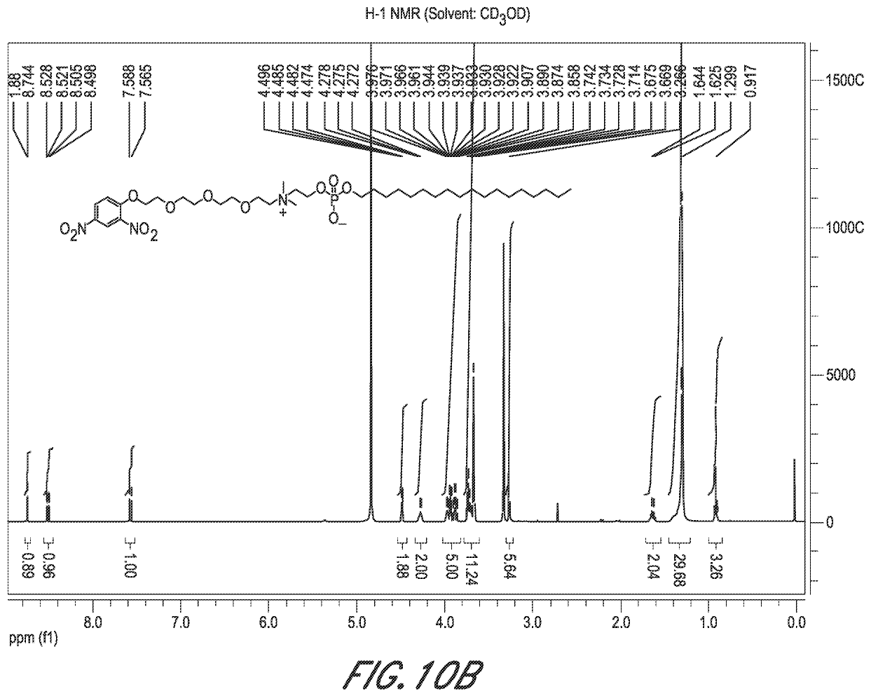

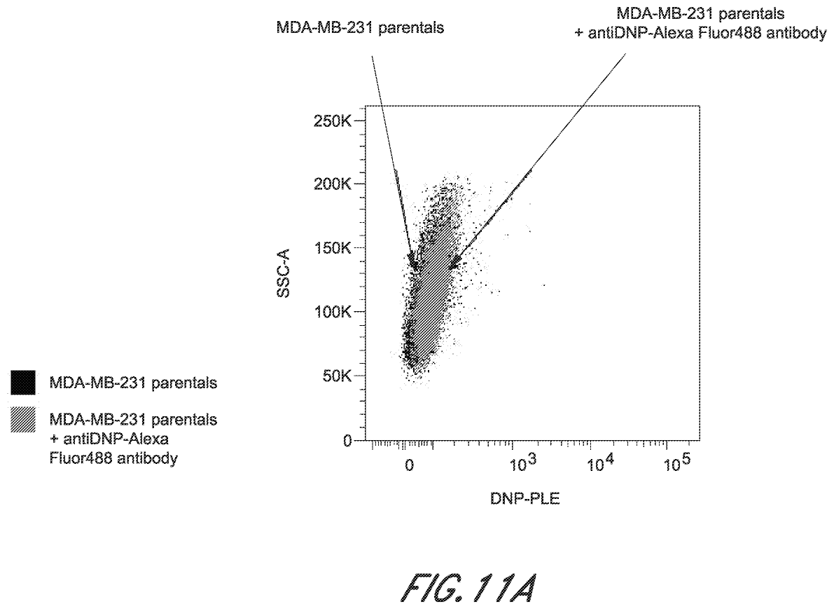

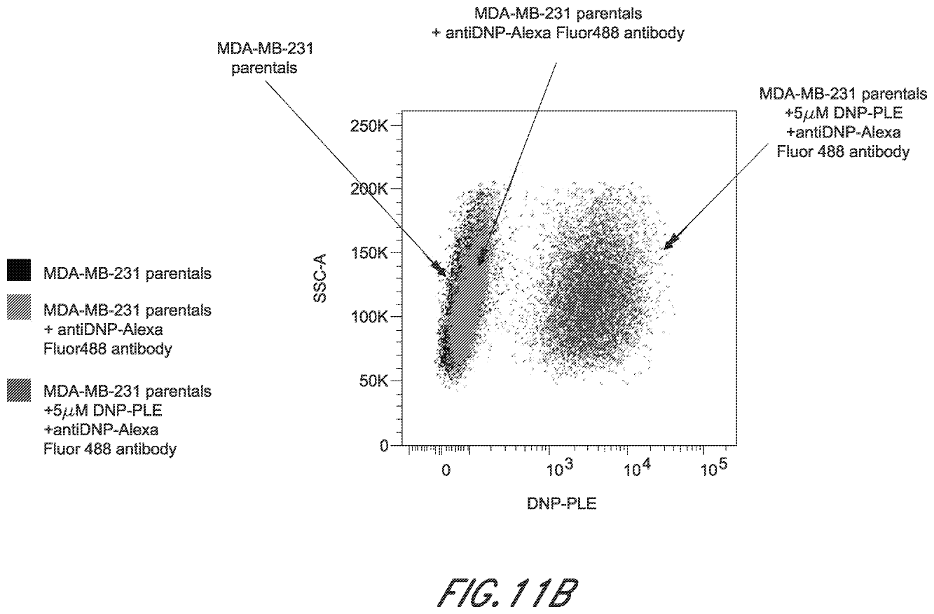

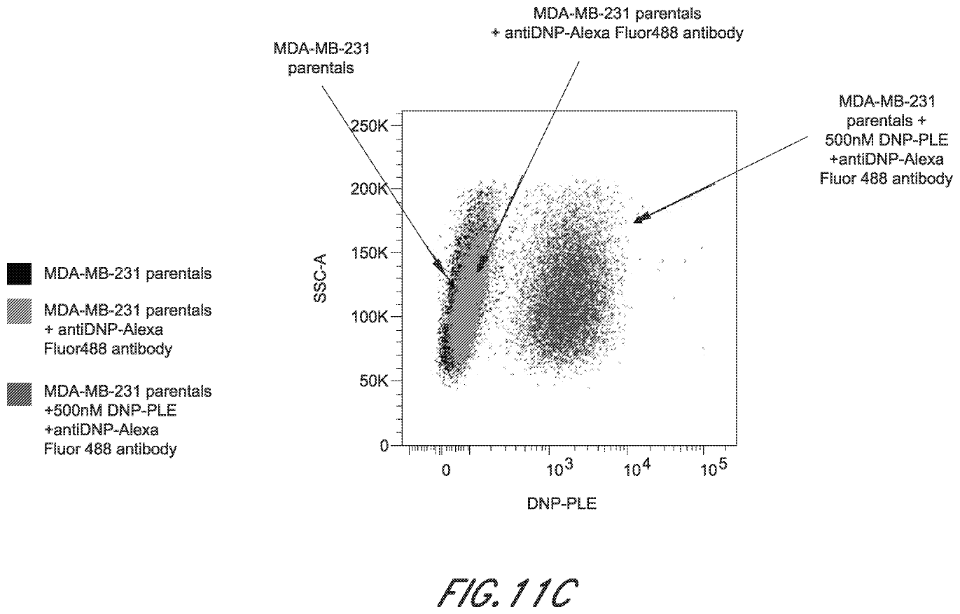

MDA-MB-231 (Adenocarcinoma) cells were incubated with DNP-PLE overnight in the presence of complete media. Cell integration of DNP-PLE was analyzed by flow cytometry post cellular staining for the exposed DNP molecules with antiDNP Alexa Fluor 488 antibody (DNP is not fluorescent). Almost no shift was seen between MDA-MB-231 parentals and MDA-MB-231 cells stained with the antiDNP-Alexa Fluor 488 antibody as shown in the control data in FIG. 11A. This is as expected since there is no DNP exposed on the surface of the MDA-MB-231 cells.

There was a clear shift from the control MDA-MB-231 parental with the MDA-MB-231 parental incubated with 5 μM DNP-PLE and stained antiDNP-Alexa Fluor 488 antibody (FIG. 11B) whereas there is a smaller shift with MDA-MB-231 parental incubated with 50 nM DNP-PLE and stained with antiDNP-Alexa Fluor 488 antibody (FIG. 11D). The difference in the shift corresponded to a difference in the amount DNP exposed on the surface of the cell for CAR T cell recognition. By changing the concentration of the chemical (DNP), the density of the hapten on the surface of the cell can also changed. The amount of DNP exposed on the surface of MDA-MB-231 parental cells incubated with 500 nM DNP-PLE was between those for 50 nM and 5 μM DNP-PLE (FIG. 11C). Histogram plots for the data in FIG. 11A-FIG. 11D are shown in FIG. 11E.

These data show that cells with tethered extracellular exposed haptens specifically DNP using DNP-PLE were successfully generated.

### Example 8—Confirming antiDNP CAR's Ability to Recognize DNP on DNP-PLE Loaded

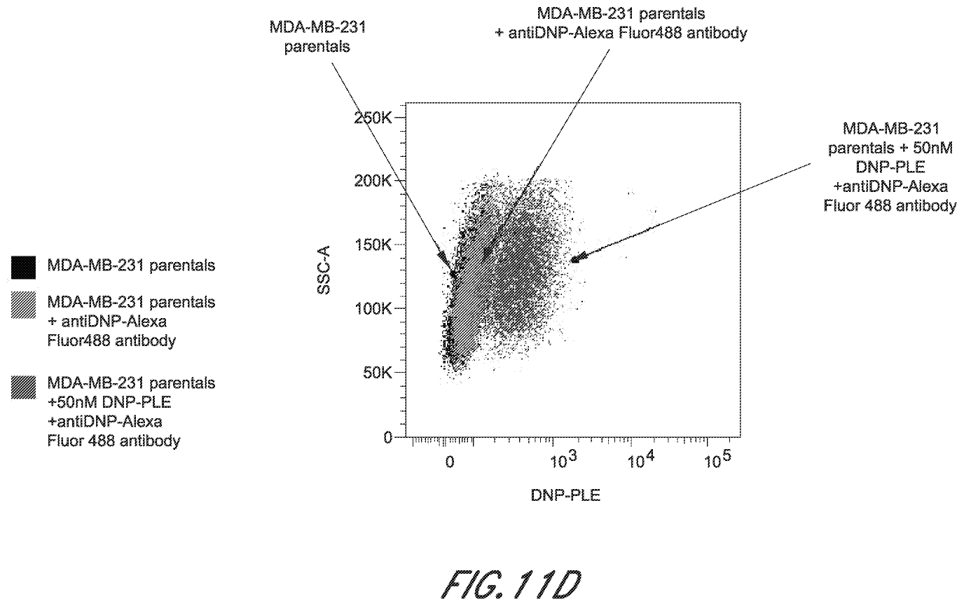

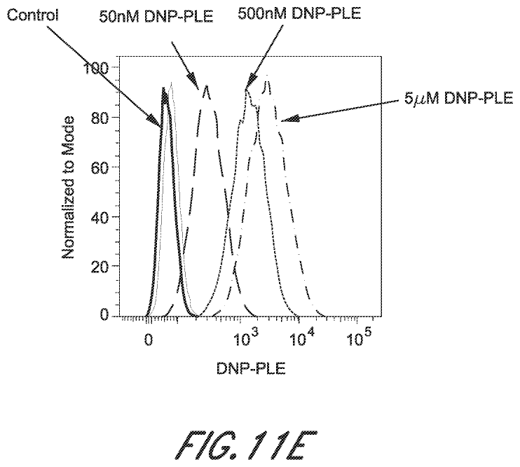

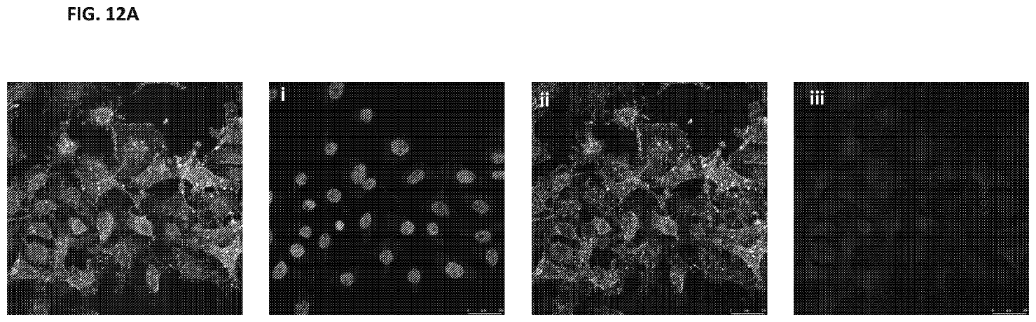

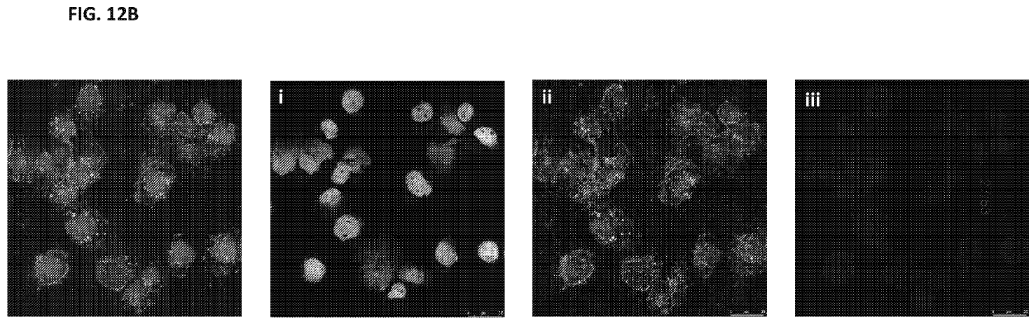

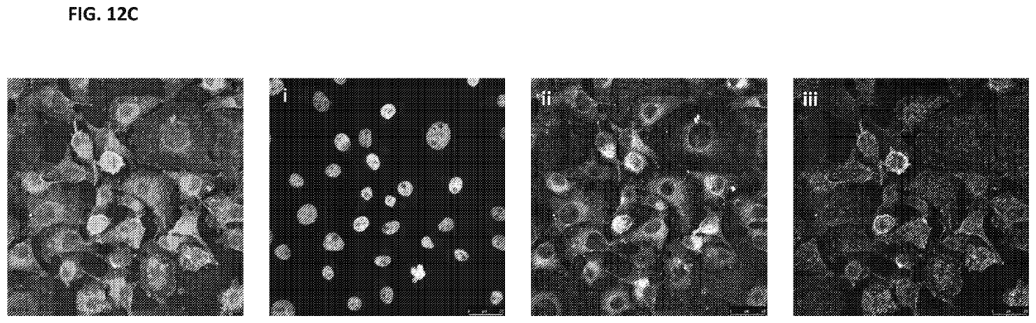

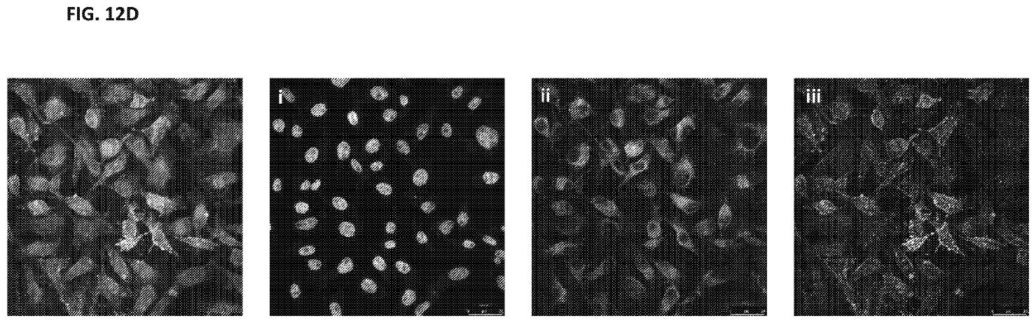

MDA-MB-231 (Adenocarcinoma) cells were loaded with 504 DNP-PLE, 1 μM DNP-PLE, or no DNP-PLE overnight, washed, and then imaged by confocal microscopy to determine where the DNP-PLE is integrated into the cells. The nucleus of the cells was stained with DAPI (i). The surface of the cell was stained with wheat germ agglutinin (WGA) (ii). Since DNP is not fluorescent, confirmed by FIG. 12B, the DNP moiety was stained with antiDNP Alexa Fluor 488 antibody (iii). The fluorescence of the antiDNP antibody is seen in (iii) and confirms that DNP-PLE integrates over the whole cell surface (FIG. 12C, FIG. 12D). These images demonstrate that the DNP moiety is accessible for binding since the antibody is able to bind. FIG. 12C is brighter than FIG. 12D, which correlates to the amount of DNP exposed on the surface. (FIG. 12A) shows the control image of MDA-MB-231 parental cells only and the antiDNP antibody is not able to bind shown by the lack of staining in the image—antiDNP AB cannot stain because there is no DNP on the surface. In FIG. 12A-FIG. 12D, the image on the left shows a full overlay confocal image of images (i)-(iv) in the respective figures. To the right of each full overlay confocal image is a grey scale version for each layer (nucleus (i), cell surface (ii), and DNP-PLE (iii)) making up the full overlay confocal image.

Thus, the ability of antiDNP CAR cells to recognize DNP on DNP-PLE loaded was confirmed.

### Example 9—Confirmation of Extracellular Accessibility of Loaded Hapten on a Cell and that the PLE was Loading in Membrane

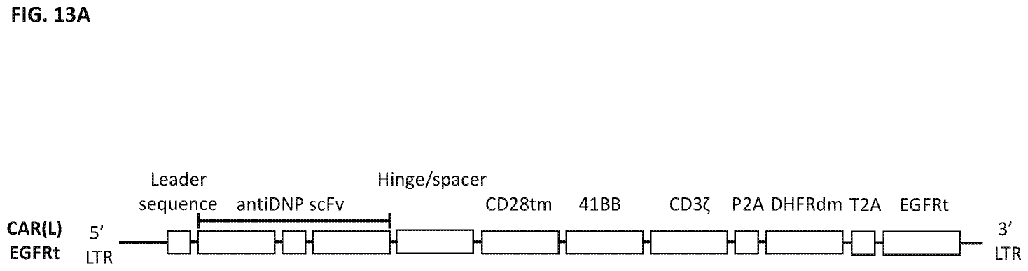

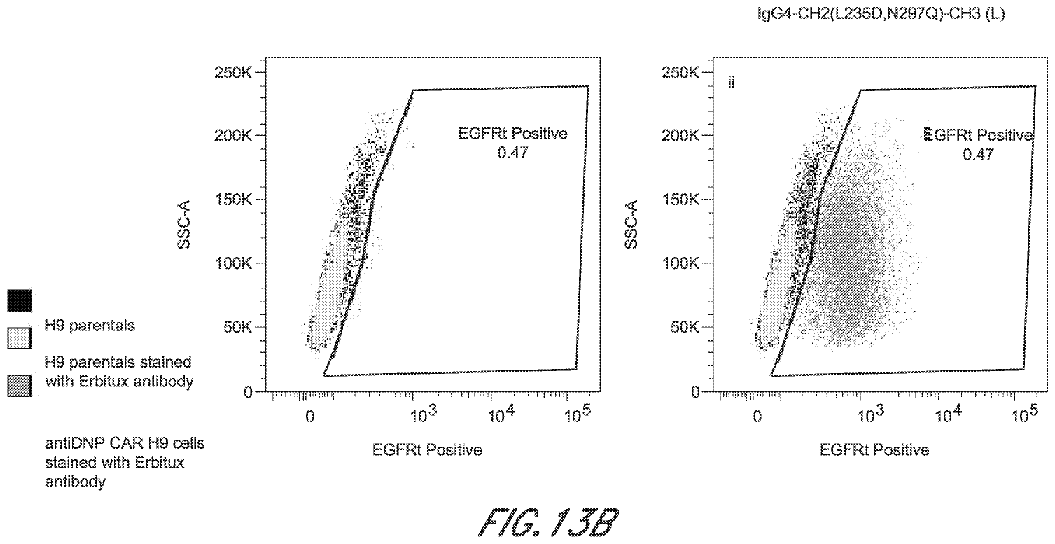

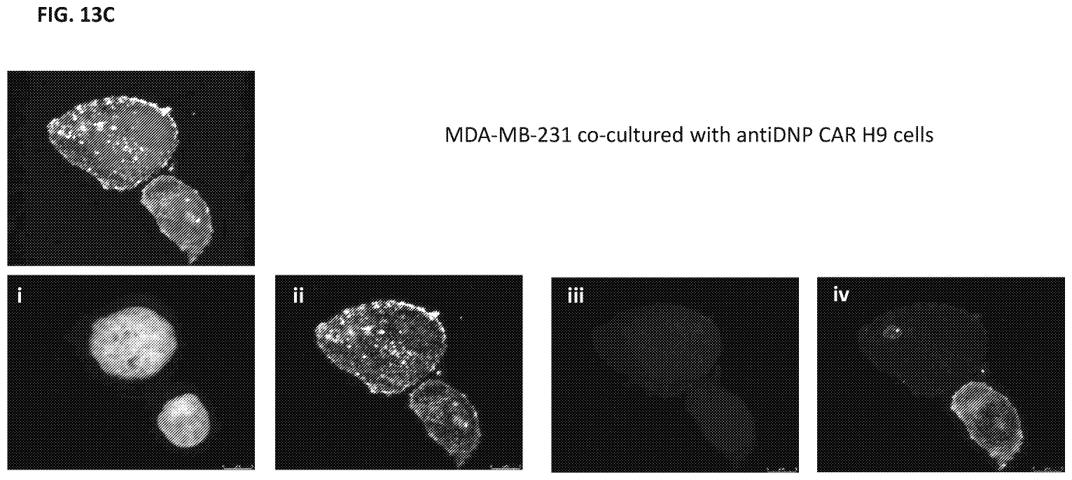

FIG. 13A shows a schematic of a second generation long CAR cassette for an antiDNP CAR. This cassette harbors a gene for a double mutant dihydrofolate reductase that allows for methotrexate selection of the CAR positive cells and the gene for EGFRt which is a surface marker that correlates to CAR positivity.

The plasmid of FIG. 13A was transduced into H9 cells (cutaneous T lymphocyte positive for CD4+ and CD3+) then methotrexated selected for a pure antiDNP CAR population. Staining for the surface marker EGFRt was used to determine the purity of the antiDNP CAR H9 cells. The cells were analyzed by flow cytometry post cellular staining and the flow plots show a 92% positive antiDNP CAR H9 populations.

The MDA-MB-231 (Adenocarcinoma) cells were loaded with or without 5 μM DNP-PLE, washed, cocultured with pure antiDNP CAR expressed in H9 cells and imaged by confocal microscopy to determine if there is recognition between the DNP exposed on the surface of the cells and the antiDNP CAR (FIG. 13C and FIG. 13D). This experiment had 2 groups: MDA-MB-231 cells cocultured with antiDNP CAR H9 cells (FIG. 13C) and MDA-MB-231 cells loaded with 504 DNP-PLE cocultured with antiDNP CAR H9 cells (FIG. 13D). The nucleus of the cells were stained with DAPI (i). The surface of the cell was stained with wheat germ agglutinin (WGA) (ii). Since DNP is not fluorescent the DNP moiety was stained with antiDNP Alexa Fluor 488 antibody ((iii), and (iv)). To determine the CAR H9 cells from the MDA-MB-231, the CAR H9 cells were stained with an antiCD3 antibody (red). Under each color image is a grey scale for each layer (nucleus (i), cell surface (ii), DNP-PLE (iii) and (iv) antiDNP CAR H9 cells) making up the full confocal image. FIG. 13C shows no binding between the targets and effectors. FIG. 13D showed an interaction between the targets and effectors. In FIG. 13C, the top left image shows full overlay confocal image of images (i)-(iv) of FIG. 13C. In FIG. 13D, the top left image shows full overlay confocal image of images (i)-(iv) of FIG. 13D. These images showed a synapse formation between the cells, thus confirming recognition of the DNP exposed on the surface of the target cell by the antiDNP CAR This is clear in FIG. 13D (iv) where the synapse is seen extending far into the target cells.

Thus, extracellular accessibility of loaded hapten on a cell and that the PLE was loading in the membrane was confirmed. The data show the generation of an antiDNP CAR and accessibility of DNP on the cell surface with the antiDNP-antibody, thus demonstrating that the antiDNP CAR can bind to DNP exposed on the surface of a cell.

### Example 10—Cytokine Production by CD19 CAR-Transduced T Cells Against Multiple Targets and Non-Autologous T-APC In Vitro

Data related to correlation of induction of CD19 CAR T cell activation to the production of specific cytokines. For cytokine production analysis, pure CD8+CD19 CAR T cell and CD8+ mock T cells [cells were used 8 days following a CD3 CD28 microbead stimulation followed by a rapid expansion protocols](effector) were plated against a panel of CD19 specific target cells at a 2:1 ratio, then incubated for 24 hours. The target cells were K562 Parental (negative control), K562 OKT3 (positive control), K562 CD19, and non-autologous clinically manufactured mixed CD4+/CD8+ truncated CD19 (CD19t) Transduced-Antigen Presenting Cells (T-APC) (positive targets, same targets used in example 11). Supernatants were analyzed for presence of cytokines. A BioPlex assay was performed to determine levels of IL-2, TNF-α and IFN-γ production. Significant amounts of cytokine were produced by CD19 CAR T cells when co-cultured with all CD19-specific target cells, including the non-autologous CD4/CD8 T-APCs. No cytokine production was detected in the non-CD19 expressing K562 Parental cell line. This experiment shows that non-autologous T-APCS can activate CD19 CAR T cells by the production of specific cytokines.

Thus, the production of cytokines by CD19 CAR-transduced T cells from non-autologous T-APCs was confirmed.

### Example 11—Autologous T-APC Activation In Vitro

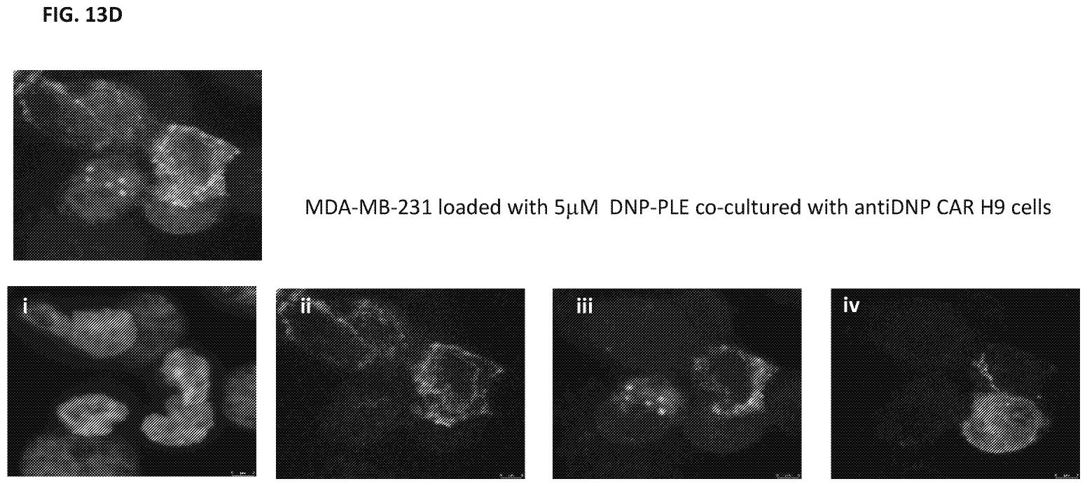

Clinically manufactured mixed CD4+/CD8+ truncated CD19 (CD19t) Transduced-Antigen Presenting Cells (T-APC) were stained and analyzed by flow cytometry for the expression of CD19t and truncated EGFR (EGFRt) on the cell surface. The CD19t T-APC are 63% positive for CD19t, and as expected, lack EGFRt expression verifying CAR negativity (FIG. 15A). Autologous CD4+ and CD8+ transduced CD19 CAR T cells were clinically manufactured and expanded via Rapid Expansion Protocol (REP) by stimulation with irradiated CD19⁺ feeder cells (TM-LCL) at a 7:1 feeder to T cell ratio in the presence of rhIL-2 and rhIL-15. Cells were stained and examined for EGFRt expression by flow cytometry on day 7 of expansion culture. Both CD4+ and CD8+ transduced CD19 CAR T cells show 99.9% positivity for EGFRt expression which correlates to CAR expression (FIG. 15B).

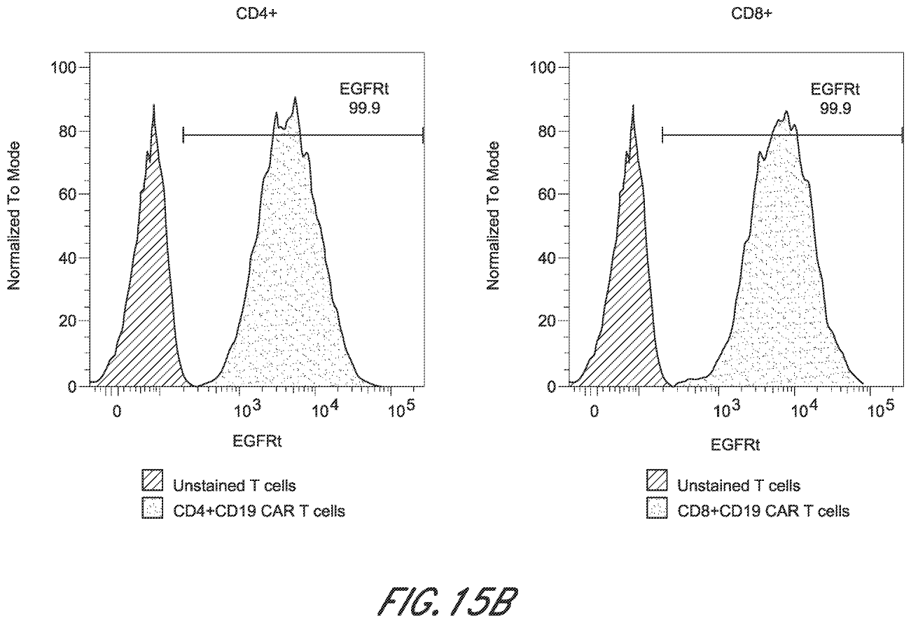

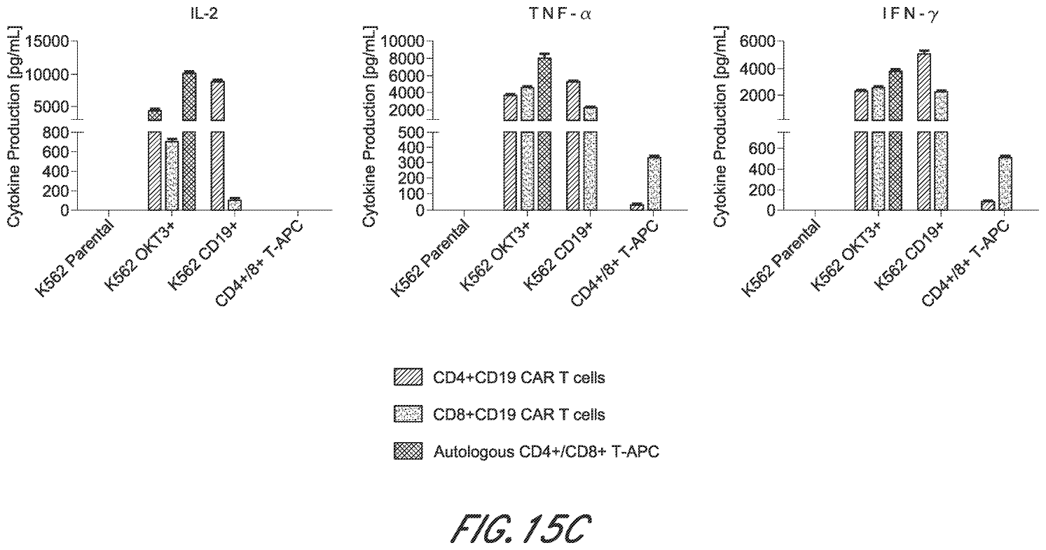

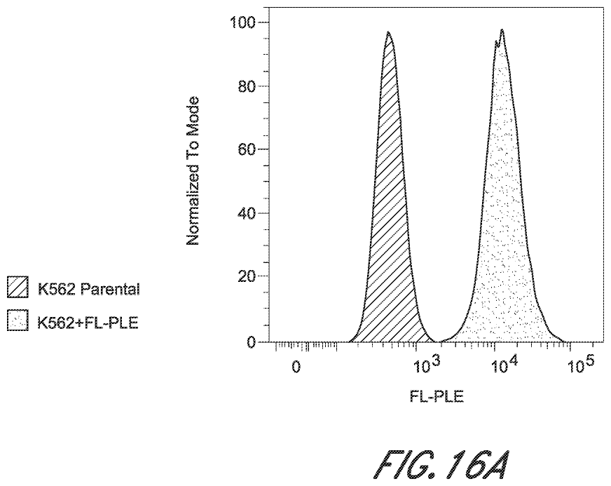

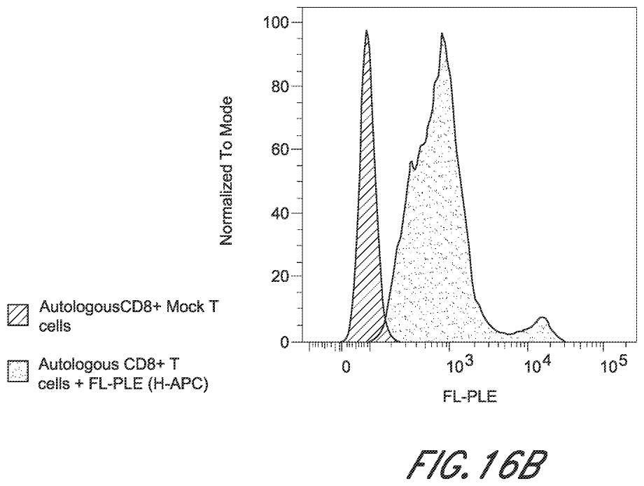

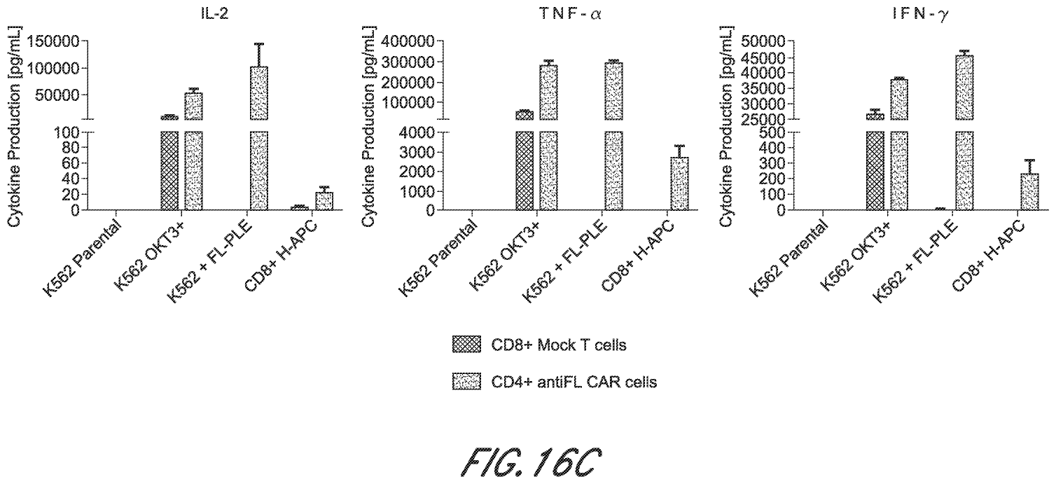

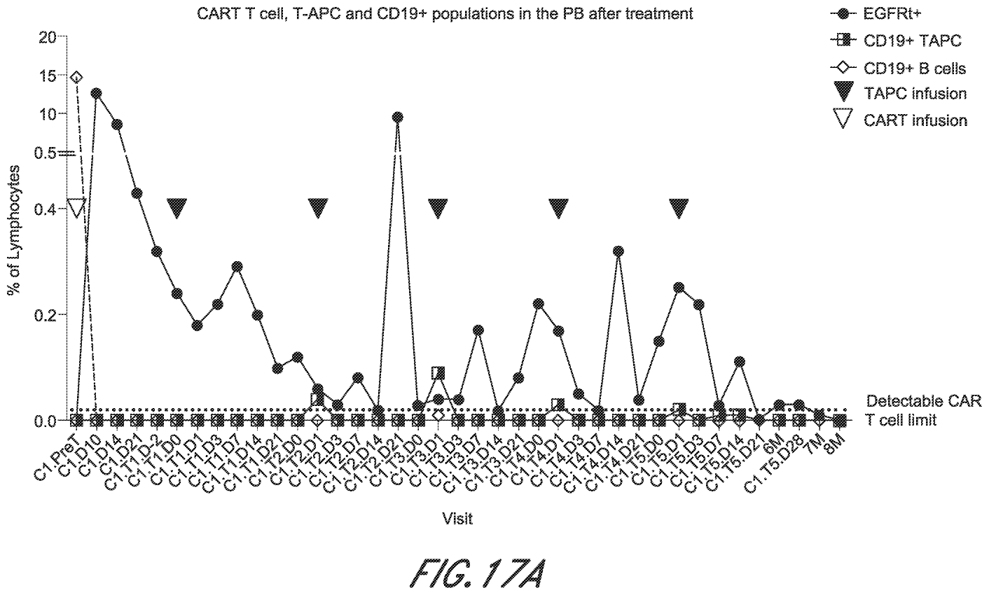

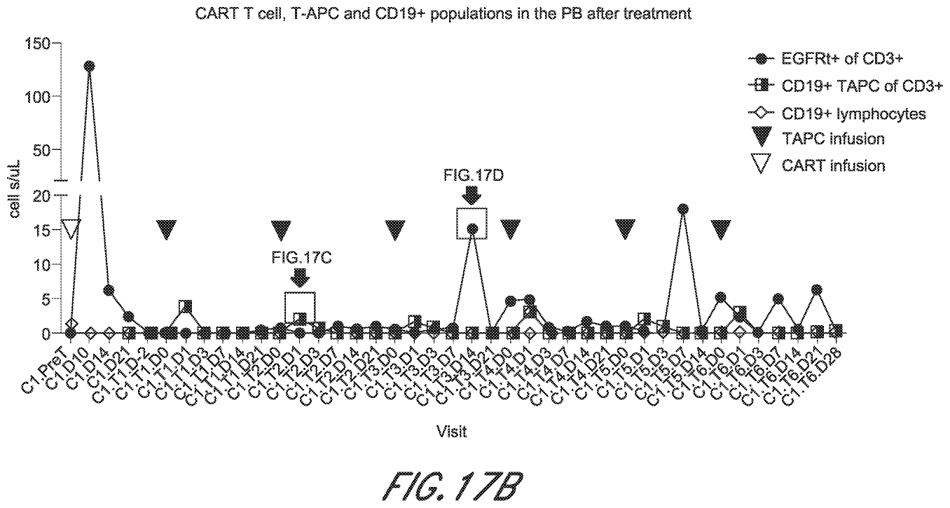

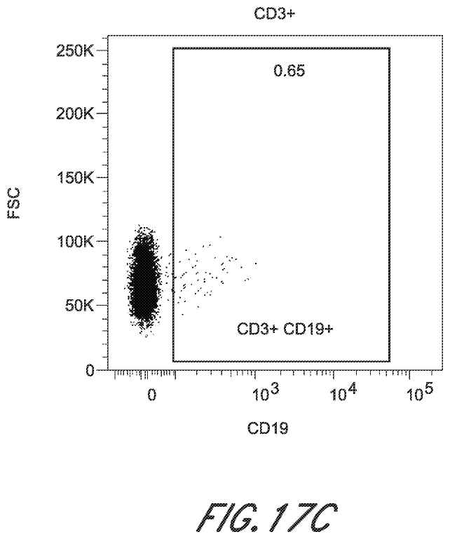

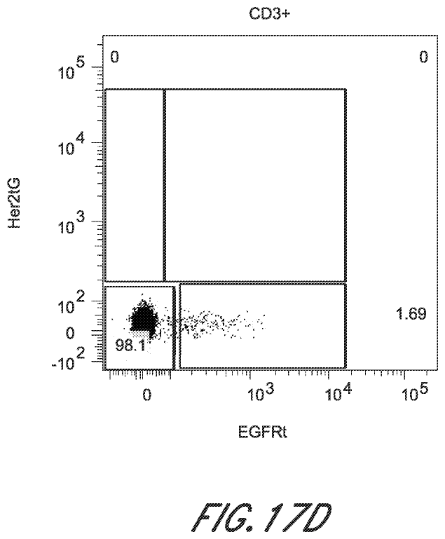

On Day 7 of expansion culture CD19t T-APC (FIG. 15A) and CD4+ and CD8+CD19 CAR T cells (FIG. 15B) were examined for cytokine production by evaluating supernatants of a 2:1 effector to target ratio after a 24 hours co-culture via a Bio-Plex Assay Kit manufactured by the Bio-Rad Corporation. CD4+ and CD8+CD19 CAR T cells and CD4+/CD8+CD19t T-APCs, were co-cultured with CD19t T-APCs, K562-CD19+(K562 parental modified to express CD19), K562-OKT3 (K562 parental modified to express agonist OKT3scFv to act as a universal positive control), and K562 Parental (negative target) cells for 24 hours. Supernatants were collected and frozen until analysis for the presence of cytokines (FIG. 15C). Following Bio-Plex assay, CD4+ and CD8+CD19 CAR T cells demonstrated antiCD19 specific cytokine production, as they were only able to produce cytokine when in the presence of the K562 CD19+ and CD19t T-APCs or K562 OKT3 positive control cell line. As expected, the CD4+/CD8+T-APCs are only able to produce cytokine in the presence of the K562 OKT3 cell line. While the co-culture of CD19t T-APCs and CD4+ and CD8+CD19 CAR T cells produce low levels of cytokine, it produces levels significant to activate the autologous CD19 CAR T cells and leads to great clinical success (See, Example 13, and FIG. 17A-FIG. 17D).

These data show that autologous T-APC could be activated in vitro.

### Example 12—Autologous Hapten-APC Activation In Vitro

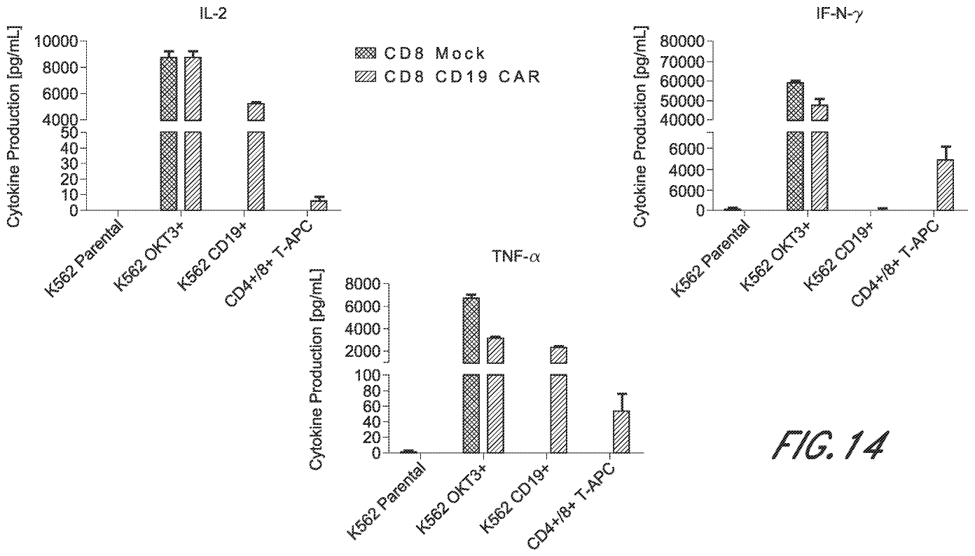

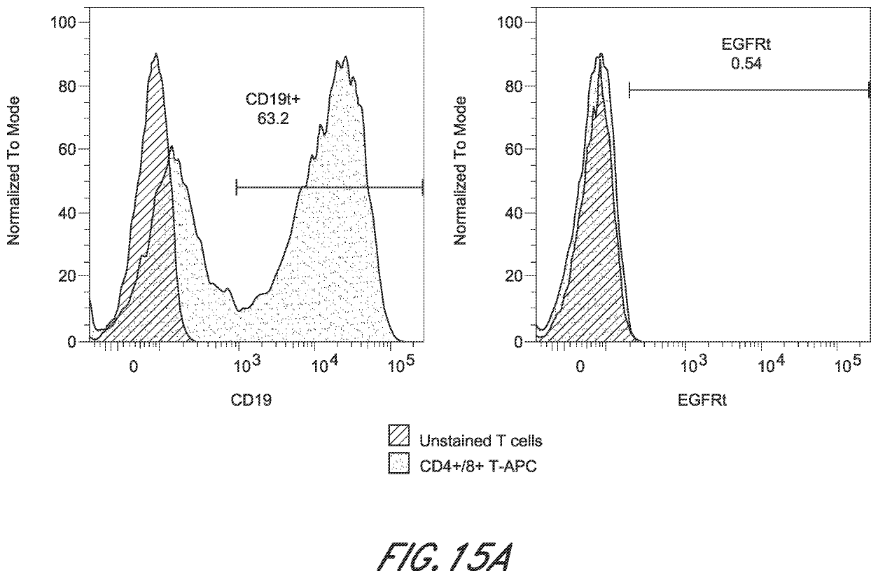

K562 leukemia cells (FIG. 16A) and primary CD8+ T cells (FIG. 16B) were incubated overnight with or without 5 μM FL-PLE and measured for fluorescence by flow cytometry. Flow cytometry analysis demonstrates FL positivity indicating successful cellular loading with the fluorescein Hapten. The ability of FL-PLE loaded cells to activate antiFL CAR T cell was measured with a cytokine release assay (FIG. 16C). Autologous CD4+ antiFL CAR effector T cells or autologous primary CD8+ T cells were co-cultured with a panel of FL-PLE loaded cells for 24 hours and supernatants were analyzed for the presence of the indicated cytokines. Both autologous CD8+ T cells and CD4+ antiFL CAR T cells were used 21 days following a CD3 CD28 microbead stimulation and two expansion protocols. Cytokine was produced by antiFL CAR T cells when co-cultured with both the K562 FL-PLE loaded cells, as well as, the autologous CD8+FL-PLE loaded cells (H-APC). As expected, no cytokine production was detected in the nonFL-PLE loaded K562 parental cell line or with the CD8+ T cells lacking CAR expression and the positive control cell line, K562 OKT3+(Non-CAR, TCR mediated activation) produced cytokine. The level of cytokine production is comparable to that from transduced-APC (T-APC) (FIG. 15A-FIG. 15C) in vitro, which have demonstrated efficacy in patients (See, Example 13, and FIG. 17A-FIG. 17D).

It is believed that the H-APC would show the same efficacy as T-APC in vivo in animal models and subjects/patients in the clinic (e.g., in clinical trials and in treatment).

These data show that autologous Hapten-APC could be activated in vitro.

### Example 13—CAR T Cell Persistence in Peripheral Blood (PB)

Persistence of CAR T cells in peripheral blood (PB) from two pediatric ALL patients following sequential T-APC dosing was investigated. Values are shown as percent of lymphocytes (FIG. 17A) or cells/μl (FIG. 17B). Patients received an infusion of CD19 CAR T cells on Day 0 (open triangle) and persistence was monitored longitudinally by surface staining for the CAR transduction marker EGFRt (filled circle) (FIG. 17A and FIG. 17B). The amount of ALL was monitored by staining for CD19+ B cells (open diamond). The patient received CD19 CAR T cell, which contain a surface marker EGFRt (filled circle) for monitoring, on day 0. The ALL quickly regressed to undetectable amounts by day 10. By Day 10 (C1.D10), CD19+ B cells were undetectable in PB and this appeared to be associated with a rapid engraftment of CAR T cells. Persistence of CAR T cells gradually declined after Day 10. As the CAR T cells were not persisting at a high enough level, in order to boost persistence, the patient received sequential doses of Transduced-Antigen Presenting Cells (T-APC) at the indicated timepoints (closed triangles). T-APC are autologous T cells engineered to express CD19 surface antigen. The patient received transduced antigen presenting cells (T-APC) where the autologous T cells express CD19 surface protein, the CAR T cell target. The T-APC express the CD3 antigen which is not found on CD19+ B cells allowing the two CD19+ populations to be differentiated. This patient received five infusions of T-APCs. After each dose of T-APC the CAR T cells expanded which in turn kept the ALL from returning. CD19+T-APC were monitored over time (half-open squares) and were distinguished from CD19+ B cells by CD3 expression. Episodic expansion of CD19 CAR T cells was observed after each infusion of T-APCs, which appeared to correlate with prolonged CD19+ B cell aplasia. Examples of the multiparameter flow of a patient's peripheral blood from the patient in FIG. 17B showing detection of CD19+T-APCs on Day 1 after dose 2 of T-APCs (FIG. 17C) and EGFR+ CAR T cells detected in PB at Day 14 after dose 3 of T-APCs (FIG. 17D). These data show that CAR T cell persist in peripheral blood (PB) of patients.

### Example 14—Labelling Peripheral Blood Mononuclear Cells with FL-PLE

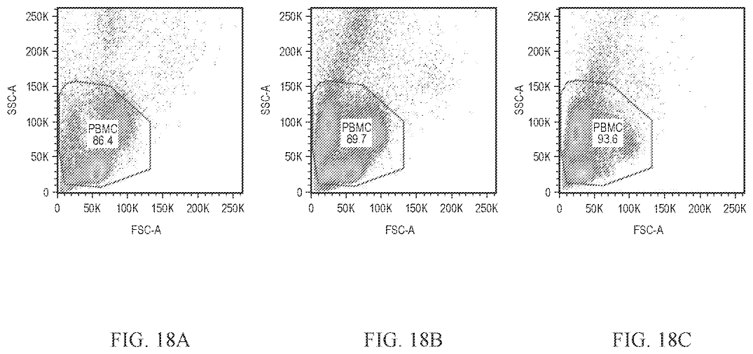

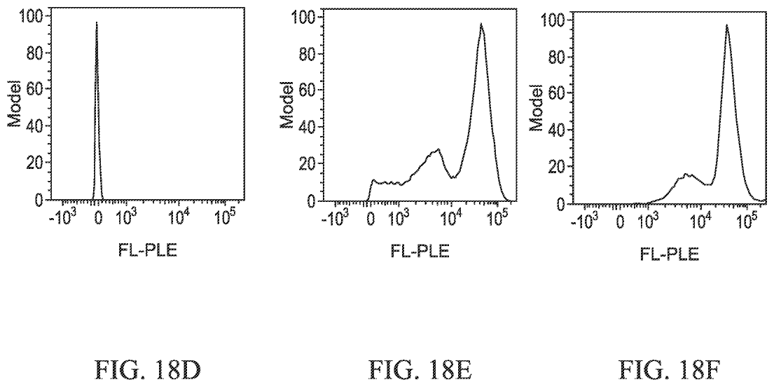

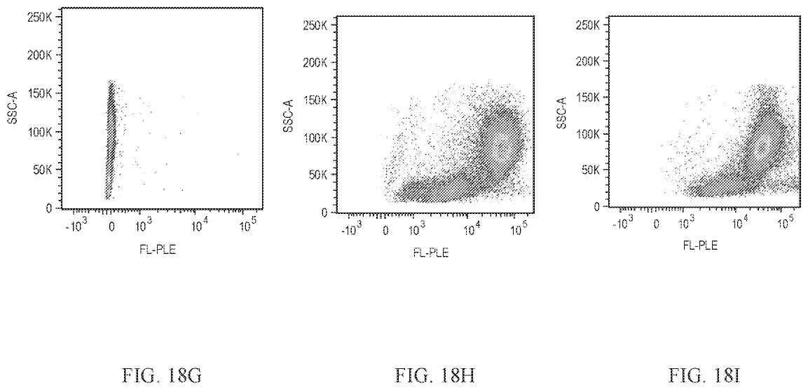

Peripheral blood mononuclear cells (PBMC) were isolated from a blood cone. The T cells were removed by a sequential CD8+ and CD4+ magnetic bead separation from the PBMC. The PMBC that had T cells removed can be seen in FIG. 18A and FIG. 18D. In particular, FIG. 18A depicts the cell population of the PBMC, and FIG. 18D depicts the amount of FL-PLE loaded onto these cells. Some of the remaining PMBC cells from the separation “PMBC (depleted of T cells)” were stain with 5 μM FL-PLE (FIG. 18B and FIG. 18E). After staining cells some cells were analyzed by flow cytometry and some FL-PLE loaded PMBC (depleted of T cells) cells were frozen in fresh freeze media. The later cells were thawed post-freeze and analyzed by flow cytometry (FIG. 18C and FIG. 18F). Both samples stained with FL-PLE (FIG. 18E and FIG. 18F) demonstrate a complete blue shift indicating the integration of FL-PLE into the PMBC (depleted of T cells) compared to unstained PMBC (depleted of T cells) where there is no blue shift (FIG. 18D). This shows that cells can undergo a freeze thaw cycle with FL-PLE integration and that FL-PLE can stain all the different cell populations of PMBC (depleted of T cells). Thus, cells can be labeled with FL-PLE, frozen and thawed, and remain labeled with FL-PLE.

### Example 15—In Vitro Expansion of Hapten Specific CAR T Cells

T cells isolated from PBMC in Example 14 were transduced with polynucleotide cassettes encoding second generation anti-fluorescein (FL) CARs comprising a long-spacer. Two different antiFL CARs were used: FITC-E2 and FITC-E2 Try100g Ala. Each cassette included a selectable gene encoding a double mutant dihydrofolate reductase for methotrexate selection of the CAR positive cells; and a gene encoding a cell surface selectable marker, a truncated CD19 polypeptide (CD19t).

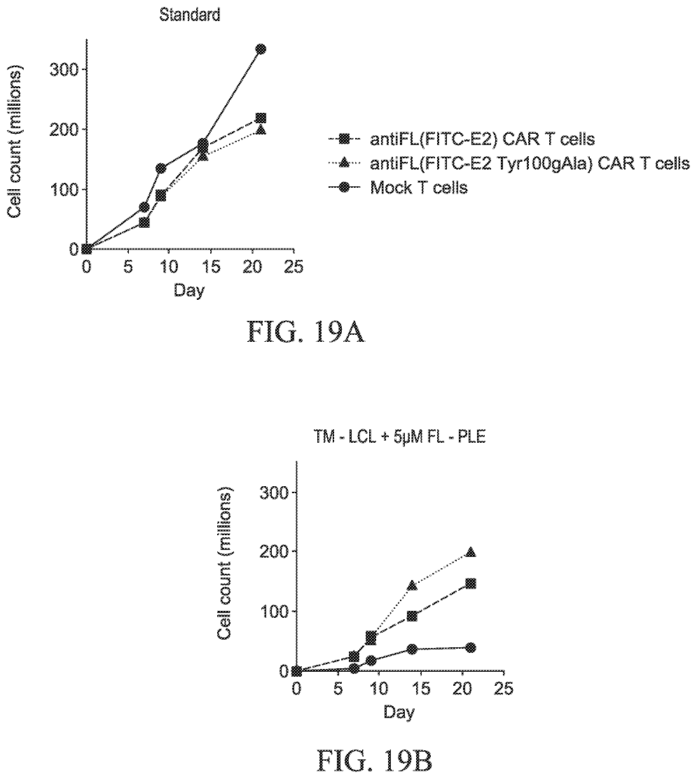

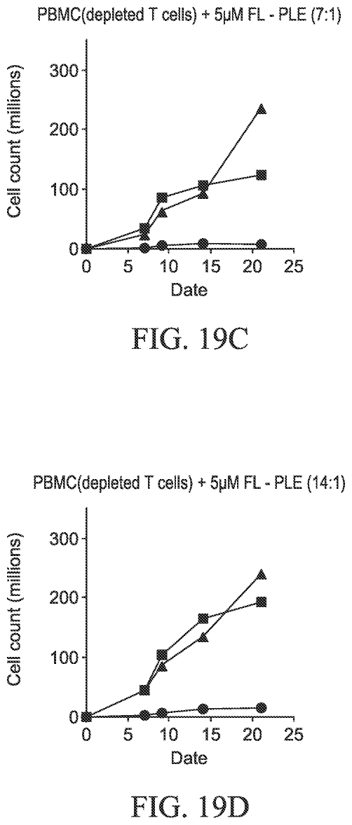

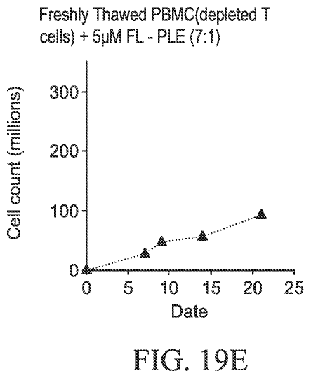

Cells were selected with methotrexate for a homogenous population of CAR positive cells. The cells underwent a standard rapid expansion protocol (REP) using irradiated TM-LCL and PBMCs (FIG. 19A). A fluorescein REP (FREP) using irradiated TM-LCL loaded with 5 μM FL-PLE at a 7:1 target to effector ratio was performed (FIG. 19B). As shown in FIG. 19C and FIG. 19D, FREP was performed using irradiated autologous PBMC (depleted of T cells) loaded with 5 μM FL-PLE at either a 7:1 or 14:1 target to effector ratio, respectively. As shown in FIG. 19E, FREP was performed using frozen, thawed, and irradiated autologous PBMC (depleted of T cells) loaded with 5 μM FL-PLE at a 7:1 target to effector ratio. Both antiFL CAR T cells and mock T cells expanded using a standard REP. However, only the antiFL CAR T cells were able to have large expansion with the FREP, especially using the autologous PBMC (depleted of T cells) loaded with 5 μM FL-PLE. This data demonstrated that autologous cells labeled with fluorescein were able to to generate expansion of anti FL CAR T cells in vitro.

### Example 16—In Vivo Expansion of Hapten Specific CAR T Cells by Hapten-APCs

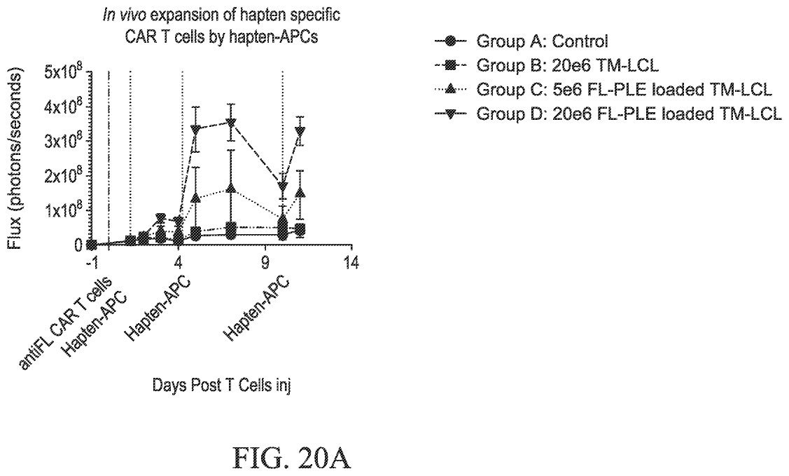

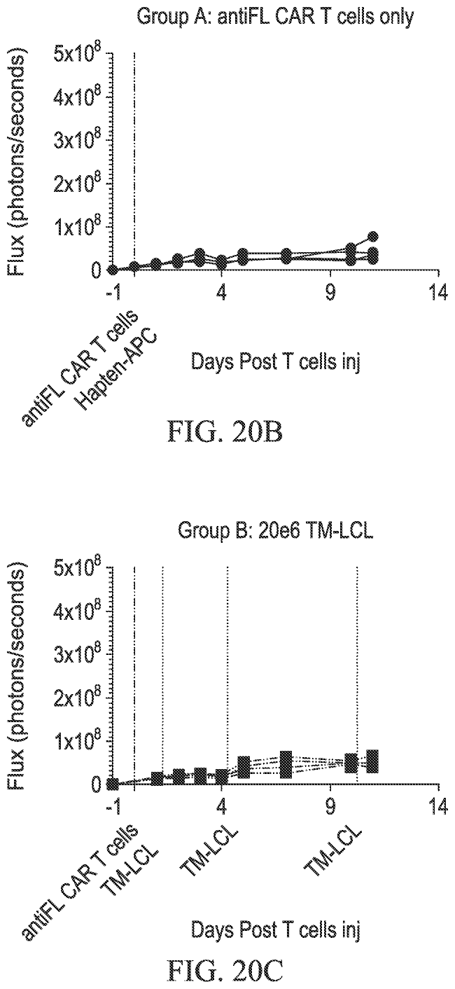

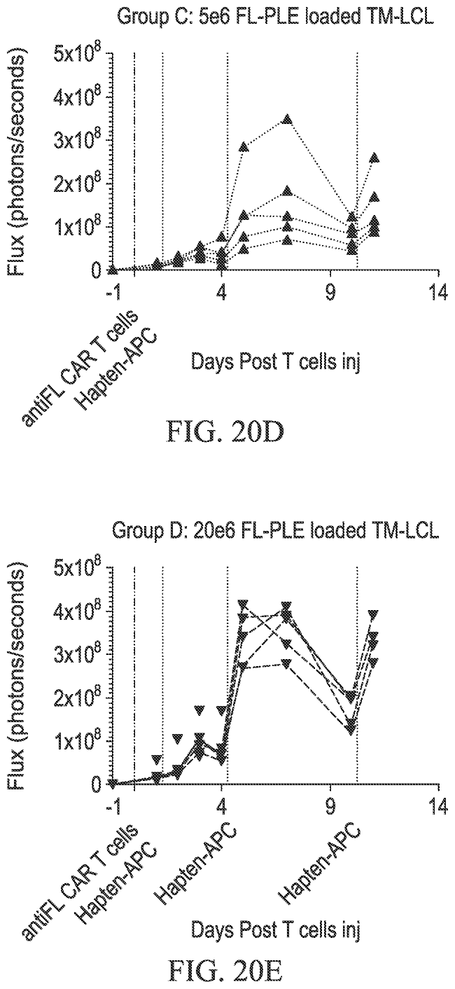

Twenty NSG mice were intravenously (IV) injected with a homogenous population of anti-fluorescein (antiFL) CAR T cells on day 0. Approximately 40% of these CAR T cells also contained a gene encoding for the fusion protein of mCherry and firefly luciferase (mCherryffLuc). The fusion protein allowed for quantitative tracking of T cell presence using bioluminescent imaging. An increase in bioluminescence signal would be indicative of an expansion of the antiFL CAR T cells. The mice were subdivided into four groups: (A) received antiFL CAR T cells only (control); (B) received antiFL CAR T cells and IV injections of 20e6 irradiated TM-LCL on days 1, 4, and 10; (C) received antiFL CAR T cells and IV injections of 5e6 irradiated TM-LCL loaded with 5 μM FL-PLE (hapten-APC) on days 1, 4, and 10; and (D) received IV injections of 20e6 irradiated TM-LCL loaded with 5 μM FL-PLE (hapten-APC) on days 1, 4, and 10. Both groups (A) and (B) had minimal expansion of the antiFL CAR T cells. The fact that group B did not undergo significant expansion showed that TM-LCL cells alone cannot expand CAR T cells. Groups (C) and (D) demonstrated expansion after each IV injection of hapten-APCs. After the second injection of hapten-APCs both groups (C) and (D) demonstrated expansion, followed by an initial regression of the antiFL CAR T cells, which was followed by another expansion of the antiFL CAR T cells subsequent to the third injection of hapten-APCs. Results are depicted in FIG. 20A-FIG. 20E. This data demonstrated the ability of hapten-APC to repeatedly expand hapten specific CAR T cells in vivo.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-114. (canceled)

115. A method of inducing expansion of a chimeric antigen receptor (CAR) T cell comprising:
incubating the CAR T cell with a hapten antigen presenting cell (H-APC), wherein the CAR T cell comprises a CAR which specifically binds to a hapten attached to the H-APC.

116. The method of claim 115, wherein the hapten is selected from a hapten listed in TABLE 1.

117. The method of claim 115, wherein the hapten is selected from fluorescein, urushiol, quinone, biotin, dinitrophenol, or a derivative thereof; and/or the CAR comprises an scFv comprising an amino acid sequence having at least 95% sequence identity to any one of SEQ ID NOs 01-06, 08 or 10.

118. The method of claim 115, further comprising attaching ex vivo the hapten to an extracellular surface of the H-APC.

119. The method of claim 118, wherein the hapten is attached to the H-APC via a phospholipid ether (PLE).

120. The method of claim 115, wherein the incubating is performed in vivo.

121. The method of claim 115, wherein the CAR T cell comprises an additional CAR which specifically binds to a cancer antigen.

122. The method of claim 115, wherein the CAR T cell and the H-APC are derived from a single subject.

123. A method of treating, inhibiting, or ameliorating a cancer in a subject comprising:
administering to the subject a chimeric antigen receptor (CAR) T cell, wherein the CAR T cell comprises a first CAR which specifically binds to a tumor specific antigen of the cancer and a second CAR which specifically binds to a hapten; and
stimulating or restimulating the CAR T cell by administering to the subject a hapten antigen presenting cell (H-APC) comprising the hapten.

124. The method of claim 123, further comprising repeating the stimulating or restimulating.

125. The method of claim 123, wherein the CAR T cell and the H-APC are derived from the subject.

126. The method of claim 123, wherein the tumor specific antigen is selected from the group consisting of CD19, CD22, HER2, CD7, CD30, B cell maturation antigen (BCMA), GD2, glypican-3, MUC1, CD70, CD33, epithelial cell adhesion molecule (EpCAM), Epidermal Growth Factor variant III, receptor tyrosine kinase-like orphan receptor 1 (ROR1), CD123, Prostate Stem Cell Antigen (PSCA), CD5, Lewis Y antigen, B7H3, CD20, CD43, HSP90, and IL13; and/or the hapten is selected from a hapten listed in TABLE 1.

127. The method of claim 123, wherein the hapten is selected from fluorescein, urushiol, quinone, biotin, dinitrophenol, or a derivative thereof; and/or the second CAR comprises an scFv comprising an amino acid sequence having at least 95% sequence identity to any one of SEQ ID NOs 01-06, 08 or 10.

128. The method of claim 123, wherein the hapten is covalently attached to the extracellular surface of the H-APC.

129. The method of claim 128, wherein the hapten is attached to the H-APC via a phospholipid ether (PLE).

130. The method of claim 123, wherein the CAR T cell is derived from a CD4+ cell, a CD8+ cell, a precursor T cell, or a hematopoietic stem cell; and wherein the H-APC is derived from a T cell or a B cell.

131. The method of claim 130, wherein the CD8+ cell is a CD8+T cytotoxic lymphocyte cell selected from the group consisting of a naïve CD8+ T cell, a central memory CD8+ T cell, an effector memory CD8+ T cell, and a bulk CD8+ T cell; or wherein the CD4+ cell is a CD4+T helper lymphocyte cell selected from the group consisting of a naïve CD4+ T cell, a central memory CD4+ T cell, an effector memory CD4+ T cell, and a bulk CD4+ T cell.

132. The method of claim 123, wherein the subject is mammalian.

133. A composition comprising:
a hapten antigen presenting cell (H-APC) comprising a hapten; and
a chimeric antigen receptor (CAR) T cell comprising a first CAR which is capable of specifically binding to a cancer antigen, and a second CAR which is specifically bound to the hapten.

134. The composition of claim 133, wherein the hapten is selected from fluorescein, urushiol, quinone, biotin, dinitrophenol, or a derivative thereof; and/or the second CAR comprises an scFv comprising an amino acid sequence having at least 95% sequence identity to any one of SEQ ID NOs 01-06, 08 or 10; and/or further comprising a cancer cell comprising the cancer antigen.
